(19) United States
(12) Patent Application Publication
(10) Pub. No.: US 2023/0077280 A1
(43) Pub. Date: Mar. 9, 2023

(54) THERAPEUTIC COMPOUNDS FOR METHODS OF USE IN INSULIN RESISTANCE

(71) Applicants: Baylor College of Medicine, Houston, TX (US); Tvardi Therapeutics, Inc., Houston, TX (US)

(72) Inventors: William E. Mitch, Houston, TX (US); Liping Zhang, Houston, TX (US); David J. Tweardy, Houston, TX (US); Imran Alibhai, Sugar Land, TX (US); Sofia de Achaval, Missouri City, TX (US)

(21) Appl. No.: 17/781,349

(22) PCT Filed: Dec. 3, 2020
(86) PCT No.: PCT/US2020/063167

§ 371 Date: May 31, 2022

Related U.S. Application Data
(60) Provisional application No. 62/943,053, filed on Dec. 3, 2019.

(51) Int. Cl. A61K 31/18; A61P 3/10; A61K 45/06

(52) U.S. Cl. CPC A61K 31/18; A61P 3/10; A61K 45/06

(57) ABSTRACT

Embodiments of the disclosure encompass methods of treating insulin resistance in an individual in need thereof by administering one or more STAT3 inhibitors to the individual. In specific aspects, the methods allow for reversing insulin resistance in individuals. The STAT3 inhibitor may be one or more particular small molecules encompassed herein.

### CROSS-REFERENCE TO RELATED APPLICATION

This application claims the benefit of U.S. Provisional Patent Application No. 62/943,053 filed on Dec. 3, 2019, the entire content of which is incorporated by reference herein.

### STATEMENT REGARDING FEDERALLY SPONSORED RESEARCH OR DEVELOPMENT

This invention was made with government support under DK37175 awarded by National Institutes of Health. The government has certain rights in the invention.

### TECHNICAL FIELD

Provided herein are methods of treating, preventing, and/or reducing the risk or severity of insulin resistance in a subject in need thereof, comprising administering to the subject a compound described herein, or a pharmaceutically acceptable salt thereof, or a pharmaceutical composition thereof.

### BACKGROUND

Insulin resistance (IR) is common in patients with chronic kidney disease (CKD)¹⁻³ even when the degree of CKD or the glomerular filtration rate (GFR) is within normal levels.⁴ IR becomes increasingly more frequent in patients with progressively lower GFR levels and is almost universal in patients with end-stage kidney failure (ESKF).^{3,5} Unfortunately, IR in CKD patients is closely associated with risk factors that contribute to cardiovascular (CV) disease, including oxidative stress,⁶ chronic inflammation,⁶ and endothelial dysfunction.⁷ Regarding the involvement of other organs, skeletal muscle represents the primary site of IR in CKD and defective intracellular signaling processes in muscle are recognized as the main defect underlying IR in CKD. Because IR is a modifiable risk factor, it is possible that its correction could potentially reduce CV morbidity and mortality but the first step in correcting IR is unveiling the molecular mechanisms responsible for the pathogenesis of CKD-related IR. Potentially, understanding mechanisms causing IR could lead to the identification of novel therapeutic targets aimed at reducing the high CV risk of CKD.

Reportedly, IR is induced by inflammation, excess glucocorticoids or myostatin expression.⁸⁻¹¹ For example, the activation of IKK-β, TGF-β1 or Smad3 signaling can serve as a link between inflammatory disorders and biologic features of IR.¹² Moreover, high glucose or high fat diets treatments induce myostatin expression in muscle and this response results in the development of IR via degradation of IRS1.¹⁰ Alternatively, the signal transducer and activator of transcription 3 (Stat3) is reportedly involved in regulating insulin signaling in several tissues. For example, Stat3 knockdown prevents the IR that occurs in hepatocarcinoma cell lines exposed to high levels of amino acids;¹³ while Stat3 activation in adipocytes has been linked to growth hormone-induced IR.¹⁴ Stat3 is activated by a range of cytokines and growth factors, including IL-6, IL-9, and epidermal growth factor. Following its nuclear translocation it binds to the promotors to regulate the expression of genes involved in inflammation, cell development, differentiation, proliferation, survival, and angiogenesis.¹⁵ Activation of Stat3 also induces the expression of SOCS proteins, which are characterized by their ability to down-regulate cytokine signaling.¹⁶ SOCS proteins also play an important role in the pathogenesis of IR because they integrate cytokine and insulin signaling processes.¹⁷ For example, overexpression of SOCS3 inhibits insulin-induced glycogen syntheses activity in myotubes and suppresses glucose uptake in adipocytes,¹⁸ whereas deletion of hepatocyte-specific SOCS3 improves insulin sensitivity in the liver.¹⁹ Mechanistically, SOCS protein activities inhibit insulin signaling by ubiquitin-conjugation and degradation of IRS1.²⁰ In skeletal muscles of Type 2 diabetic (T2D) patients, Stat3 was found to be constitutively phosphorylated.²¹ The major remaining questions is whether inhibition of Stat3 activation will improve insulin signaling in muscles.

In skeletal muscles, Atrogin-1 has been identified as a muscle-specific E3 ubiquitin ligase; it is used as a marker of the degree of muscle proteolysis that occurs in models of skeletal muscle atrophy. Atrogin-1 also is a muscle-specific F-box protein (Fbxo32).22,23 F-box proteins are key components of the SCF (Skp1-Cullin1-Fbox protein) complex. F-box proteins interact with Skp1, using the F-box domain, and proteins to be ubiquitin-conjugated.²⁴ Specifically, there are over 70 genes encoding F-box containing proteins; they exert E3 ubiquitin ligase activities that participate in the regulation of cell cycle and signal transduction functions.²⁵ Recently, Fbxo40 has been identified as another muscle-specific F-box protein,²⁶ but its role in the functions of muscles has not been defined. There are a few reports indicating that Fbxo40 expression is muscle-specific and that its expression is upregulated during differentiation. Thus, knockdown of Fbxo40 in muscles induces dramatic hypertrophy of myofibers.²⁵ Fbxo40 expression also was found to be upregulated in skeletal muscles following denervation,²⁶ while mice null for Fbxo40 exhibited enhanced body and muscle sizes during the growth phase when serum IGF1 levels are elevated ²⁵. Together, these reports suggest that Fbxo40 could play important roles when muscle atrophy is developing, but this is speculative because the factors or the mechanisms regulating Fbxo40 expression are unknown.

Insulin receptor substrate (IRS) proteins mediate insulin receptor tyrosine kinase signaling. Reduced levels of IRS1 expression and protein have been linked to the development of both IR and T2D in humans.²⁷ In mice, genetic disruption of IRS1 is associated with impaired insulin-stimulated glucose disposal in vivo and glucose transport in vitro.²⁸⁻³⁰ These responses are relevant, because IRS proteins activate PI3K which recruits Akt to the plasma membrane leading to it phosphorylation and activation. The involvement of p-Akt in metabolic regulation is multifold: downstream substrates can play key roles in the response of cells to IR/IGF-1R signaling including the Akt Substrate of 160 kD (AS160), the FOXO transcription factors, and mTORC1. Akt activation is also required for translocation of the glucose transporter GLUT4 to the plasma membrane to transfer glucose to muscle or adipose cells (FIG. 5).

The present disclosure provides solutions to a long-felt need in the art of IR and associated health conditions.

### SUMMARY

The present disclosure is directed to compounds, compositions, and methods related to treating, preventing, and/or reducing the risk or severity of insulin resistance (IR) in an individual in need thereof. In particular embodiments, the IR is related to CKD, although in other embodiments, the IR is not related to CKD and/or the individual does not have CKD.

In some embodiments, the present disclosure concerns inhibition of mechanisms that directly or indirectly result in IR, including in CKD. For example, the present disclosure provides compounds and compositions thatare useful in inhibiting Stat3 and thus treating IR. In some embodiments, the inhibition of Stat3 results in an improvement of IR, including IR associated with CKD. In some embodiments, the Stat3 inhibitor (e.g., a Stat3 inhibitor described herein) is useful in reversing IR in patients that have IR, and in particular embodiments the Stat3 inhibitor directly inhibits Stat3 to result in such reversal.

Embodiments of the disclosure also provide for mouse models for insulin resistance that are mice with CKD or are mice that are fed a high fat diet (HFD). Such models are useful because the level of activated Stat3 (Stat3 phosphorylated on tyrosine 705, p-Stat3) is increased in skeletal muscles of CKD or HFD mice. Such models were useful for characterizing a new pathway for the long standing problem of IR in CKD.

In particular embodiments, the Stat3 inhibitors to be utilized with the methods disclosed herein are small molecule inhibitors of Stat3 that improves insulin signaling in an individual (e.g., in mice or human) with or without CKD or HFD. The Stat3 inhibitor may be formulated in any manner that allows for therapeutically effective treatment. Individuals being treated for insulin resistance or CKD may or may not be given an additional treatment for the respective insulin resistance or CKD. In particular embodiments, the Stat3 inhibitor compounds and compositions encompassed herein are used for treatment of Type II Diabetes, obesity, and/or CV disease. In some embodiments, IR develops as a complication of several illnesses characterized by the presence of inflammation, acute and chronic kidney failure (e.g., in Type II diabetes, obesity and/or cardiovascular diseases).

Embodiments of the disclosure include methods of treating insulin resistance in an individual in need thereof, comprising administering to the individual a therapeutically effective amount of one or more inhibitors of STAT3. Embodiments of the disclosure include methods of treating, preventing, or reducing the risk or severity of insulin resistance or a condition associated with insulin resistance in an individual in need thereof, comprising administering to the individual a therapeutically effective amount of one or more inhibitors of signal transducer and activator of transcription 3 (STAT3). Embodiments of the disclosure include methods of treating, preventing, or reducing the risk or severity of diabetes in an individual in need thereof, comprising administering to the individual a therapeutically effective amount of one or more inhibitors of signal transducer and activator of transcription 3 (STAT3). Embodiments of the disclosure include methods of treating, preventing, or reducing the risk or severity of metabolic syndrome in an individual in need thereof, comprising administering to the individual a therapeutically effective amount of one or more inhibitors of signal transducer and activator of transcription 3 (STAT3). The IR in the individual may be associated with inflammation. The individual may have chronic kidney disease. In specific embodiments, the individual does not have cachexia or muscle wasting. In particular embodiments, the individual is a mammal, such as a human, dog, cat, horse, cow, pig, sheep, or goat. The inhibitor of STAT3 may be a small molecule, in specific cases, and in some embodiments the inhibitor of STATS is one or more inhibitors from any one of Tables 1-7, or a pharmaceutically acceptable salt thereof. Methods include the further step of administering to the individual an effective amount of an additional therapy for IR or an associated medical condition thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIG. 1A shows percentage changes in body weight in mice with CKD from day 1 to day 12 of TTI-101 or D5W diluent injections. (*, p<0.05 vs. CKD-D5W, n=10 mice).

FIG. 1B shows fasting blood glucose (16 hr fasting) in mice with CKD. (*, p<0.05 vs. Sham-D5W, #p<0.05 vs. CKD-D5W n=10 mice).

FIG. 1C shows assessment of glucose tolerance in mice with CKD. (*, p<0.05 vs. CKD-D5W, n=10 mice).

FIG. 1D shows lysates of gastrocnemius muscles in mice with CKD subjected to western blotting. Image quantification is shown in the right panel (*, p<0.05 vs. CKD-D5W, n=10 mice).

FIG. 2A shows Stat3 binding site in the promoter region of mouse Fbxo40.

FIG. 2B shows that CHIP assay uncovered enriched Stat3 relative to IgG in the promoter of Fbxo40 in C2C12 myotube expressing GFP (control) or overexpressing Stat3C (*, p<0.05 vs. GFP, n=3 repeat).

FIG. 2C shows Stat3C stimulates Fbxo40 promoter activity (*, p<0.05 vs. cDNA3, n=3 repeat).

FIG. 2D shows C2C12 cells transfected with Stat3C increased Fbxo40 proteins.

FIG. 2E shows western blotting for cell lysates of C2C12 myotubes treated with 100 ng/ml IL-6 for 24 hr.

FIG. 2F shows C2C12 cells were transfected with SiRNA of control or Fbxo40 (for 24 hr) and were differentiation into myotubes (48 hr), then treated with 100 ng/ml IL-6 for 24 h. Cell lysates were subjected to western blotting.

FIG. 2G shows mRNAs of Fbxo40 evaluated in TA muscles of mice (*, p<0.05 vs. Sham-D5W, #p<0.05 vs. CKD-D5W, n=10 mice).

FIG. 2H shows mRNAs of IRS1 evaluated in TA muscles of mice (*, p<0.05 vs. Sham-D5W, #p<0.05 vs. CKD-D5W, n=10 mice).

FIG. 2I shows muscle lysates from CKD mice that were treated with or without TTI-101 were subjected for western blotting to evaluate protein levels of Fbxo40.

FIG. 2J. quantification of images in FIG. 21 (*, p<0.05 vs. CKD-D5W, n=10 mice).

FIG. 3A shows muscle lysates from mice fed the HFD for two weeks were subjected to western blotting for p-Stat3. Quantification of images are shown in the right panel (*, p<0.05 vs. RD, n=10 mice).

FIG. 3B shows mRNAs of Fbxo40 from tibialis anterior muscles obtained after feeding mice with the HFD for two weeks (*, p<0.05 vs. RD, n=10 mice).

FIG. 3C shows mRNAs of Fbxo32 from tibialis anterior muscles obtained after feeding mice with the HFD for two weeks (*, p<0.05 vs. RD, n=10 mice).

FIG. 3D shows mRNAs of MuRF-1 from tibialis anterior muscles obtained after feeding mice with the HFD for two weeks (*, p<0.05 vs. RD, n=10 mice).

FIG. 3E shows 12 weeks of HFD feeding induced obesity in mice (*, p<0.05 vs. RD, n=10 mice).

FIG. 3F shows 12 weeks of HFD feeding induced glucose intolerance (3F) in mice (*, p<0.05 vs. RD, n=10 mice).

FIG. 3G shows TTI-101 treatment of HFD mice decreased fasting glucose level in mice (*, p<0.05 vs. HFD-D5W or HFD-before treatment, n=10 mice).

FIG. 3H shows TTI-101 treatment of HFD-fed mice improved their glucose tolerance (*, p<0.05 vs. HFD-D5W, n=10 mice).

FIG. 31 shows TTI-101 treatment of HFD-fed mice improved their insulin tolerance (*, p<0.05 vs. HFD-D5W, n=10 mice).

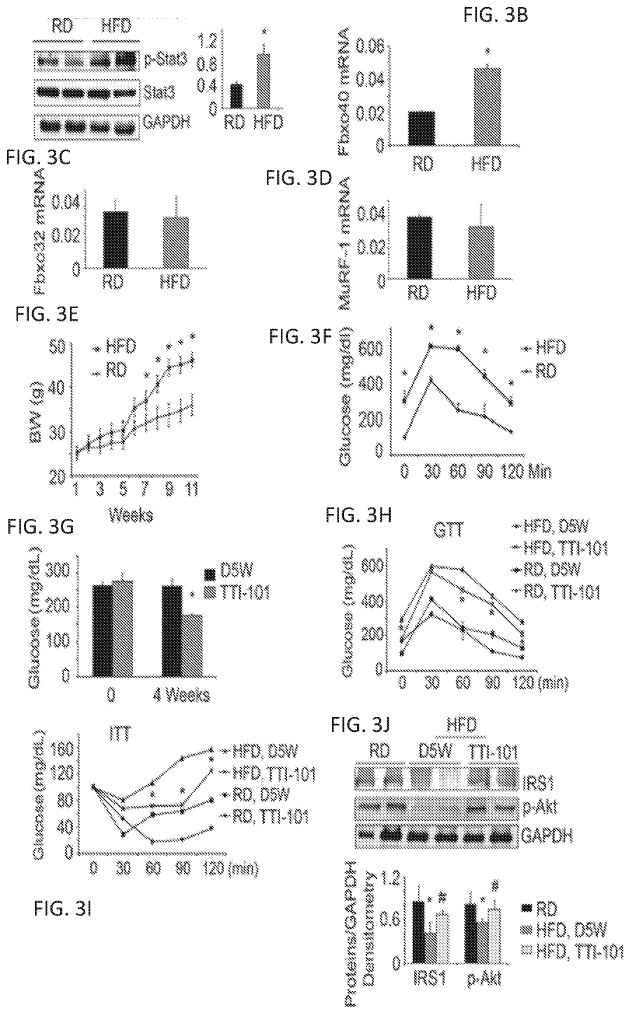

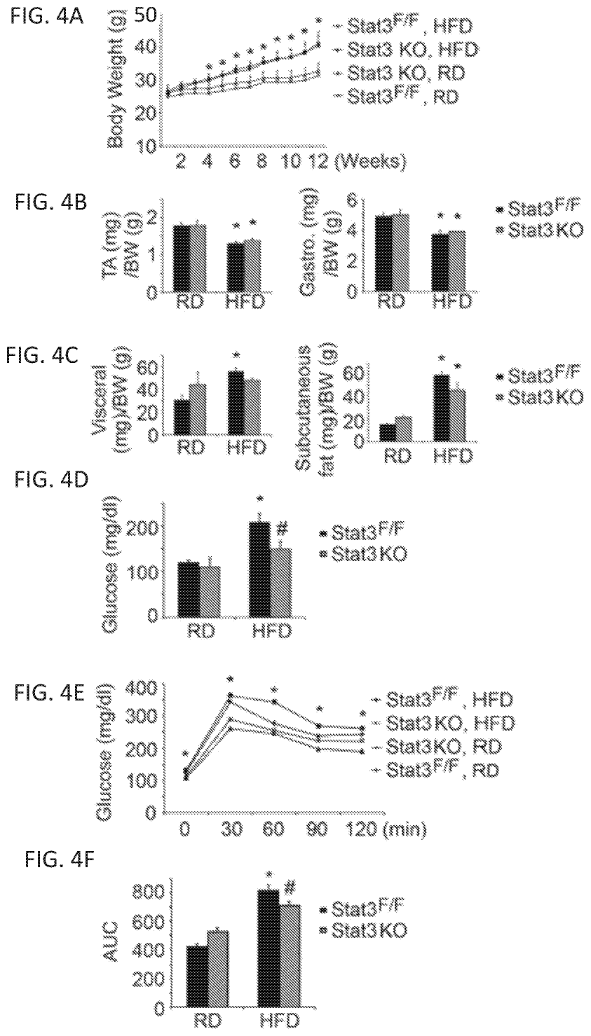

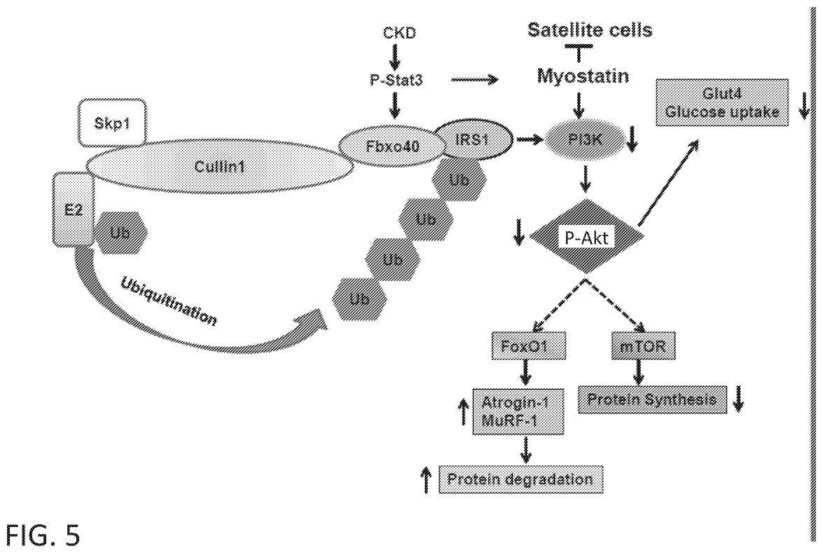

FIG. 3J shows the muscle lysates of HFD mice treated with/without TTI-101 subjected for western blotting to evaluate IRS1 or p-Akt proteins. Quantification of images is shown in lower panel (*, p<0.05 vs. RD, #, p<0.05 vs. HFD-D5W, n=10 mice).

FIG. 4A shows body weights during 12 weeks HFD feeding (*; p<0.05 vs. RD, n=10 mice).

FIG. 4B shows muscle weights in mice after 16 weeks HFD (*, p<0.05 vs. RD, n=10 mice).

FIG. 4C shows adipose tissues mass after 16 weeks HFD (*, p<0.05 vs. RD, n=10 mice).

FIG. 4D shows HFD-Stat3 KO mice decreased fasting glucose level in mice (*, p<0.05 vs. RD-Stat3f/f, #p<0.05 vs. HFDStat3f/f, n=10 mice).

FIG. 4E shows glucose tolerance test in mice after 16 weeks HFD (*, p<0.05 vs. HFD-Stat3f/f, n=10 mice).

FIG. 4F shows AUC calculated from 4E (*, p<0.05 vs. RD-Stat3f/f, #p<0.05 vs. HFD-Stat3f/f n=10 mice).

FIG. 5 shows pathways for CKD stimulating Stat3 leading to loss of muscle mass and IR. CKD-induces IL-6 production leading to stimulation of Stat3. Stat3 activation induces myostatin production. The increase in myostatin impairs satellite cell function. Myostatin also increases Smad2/3 phosphorylation, suppressing Akt phosphorylation, resulting in activation of the ubiquitin-proteasome system (UPS) and muscle atrophy. Stat3 also stimulates Fbxo40 expression cause ubiquitination and degradation of IRS1 leading to IR.

### DETAILED DESCRIPTION

### Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the invention may consist of or consist essentially of one or more elements, method steps, and/or methods of the invention. It is contemplated that any method, compound, or composition described herein can be implemented with respect to any other method, compound, or composition described herein.

The term “inhibitor” as used herein refers to one or more molecules that interfere at least in part with the activity of Stat3 to perform one or more activities, including the ability of Stat3 to bind to a molecule and/or the ability to be phosphorylated.

The phrase “therapeutically effective amount” as used herein means that amount of a compound, material, or composition comprising a compound of the present invention that is effective for producing some desired therapeutic effect, e.g., treating (i.e., preventing and/or ameliorating) cancer in a subject, or inhibiting protein-protein interactions mediated by an SH2 domain in a subject, at a reasonable benefit/risk ratio applicable to any medical treatment. In one embodiment, the therapeutically effective amount is enough to reduce or eliminate at least one symptom. One of skill in the art recognizes that an amount may be considered therapeutically effective even if the cancer is not totally eradicated but improved partially. For example, the spread of the cancer may be halted or reduced, a side effect from the cancer may be partially reduced or completed eliminated, life span of the subject may be increased, the subject may experience less pain, and so forth.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “mammal” is an appropriate subject for the method of the present invention. A mammal may be any member of the higher vertebrate class Mammalia, including humans; characterized by live birth, body hair, and mammary glands in the female that secrete milk for feeding the young. Additionally, mammals are characterized by their ability to maintain a constant body temperature despite changing climatic conditions. Examples of mammals are humans, cats, dogs, cows, mice, rats, and chimpanzees. Mammals may be referred to as “patients” or “subjects” or “individuals”.

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄) alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c}, and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle, and aryl can themselves be optionally substituted.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c}, and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary such groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. “Substituted alkynyl” refers to an alkynyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c}, together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “cycloalkyl” refers to a fully-saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c}, and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c}, and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl, or naphthyl. Where containing two or more aromatic rings (bicyclic, etc.), the aromatic rings of the aryl group may be joined at a single point (e.g., biphenyl), or fused (e.g., naphthyl, phenanthrenyl, and the like). “Substituted aryl” refers to an aryl group substituted by one or more substituents, preferably 1 to 3 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c}, and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle, and aryl substituents can themselves be optionally substituted.

The term “carbocycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl, or naphthyl. The term “carbocycle” encompasses cycloalkyl, cycloalkenyl, cycloalkynyl, and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl, and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 4 to 7 membered monocyclic, 7 to 11 membered bicyclic, or 8 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms, and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl], or furo[2,3-b]pyridinyl), dihydroisoindolyl, dihydroquinazolinyl (such as 3,4-dihydro-4-oxo-quinazolinyl), triazinylazepinyl, tetrahydroquinolinyl, and the like. Exemplary tricyclic heterocyclic groups include carbazolyl, benzidolyl, phenanthrolinyl, acridinyl, phenanthridinyl, xanthenyl, and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c}, and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The term “alkylamino” refers to a group having the structure —NHR′, wherein R′ is hydrogen, alkyl or substituted alkyl, or cycloalkyl or substituted cyclolalkyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each independently alkyl or substituted alkyl, cycloalkyl or substituted cycloalkyl, cycloalkenyl or substituted cyclolalkenyl, aryl or substituted aryl, or heterocylyl or substituted heterocyclyl, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-trianolyl, and tetrazolyl.

The terms “halogen” or “halo” refer to chlorine, bromine, fluorine, or iodine.

Unless otherwise indicated, any heteroatom with unsatisfied valences is assumed to have hydrogen atoms sufficient to satisfy the valences.

The compounds of the present disclosure may form salts which are also within the scope of this disclosure. Reference to a compound of the present disclosure is understood to include reference to salts thereof, unless otherwise indicated. The term “salt(s),” as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the present invention may be formed, for example, by reacting a compound described herein with an amount of acid or base, such as an equivalent amount, in a medium such as one in which the salt precipitates or in an aqueous medium followed by lyophilization.

The compounds of the present disclosure which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentane propionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present disclosure which contain an acidic moiety, such but not limited to a carboxylic acid, may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine, and the like. Basic nitrogen-containing groups may be quaternized with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), aralkyl halides (e.g., benzyl and phenethyl bromides), and others.

Compounds of the present disclosure, and salts or solvates thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives, or separation by chiral column chromatography. The individual optical isomers can be obtained from the racemates by any suitable method, including without limitation, conventional methods, such as, for example, salt formation with an optically active acid followed by crystallization.

Compounds of the present disclosure are, subsequent to their preparation, preferably isolated and purified to obtain a composition containing an amount by weight equal to or greater than 90%, for example, equal to greater than 95%, equal to or greater than 99% of the compounds (“substantially pure” compounds), which is then used or formulated as described herein. Such “substantially pure” compounds of the present invention are also contemplated herein as part of the present invention.

All configurational isomers of the compounds of the present disclosure are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specification, groups and substituents thereof may be chosen to provide stable moieties and compounds.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry,” Thomas Sorrell, University Science Books, Sausalito (1999), the entire contents of which are incorporated herein by reference.

Certain compounds of the present disclosure may exist in particular geometric or stereoisomeric forms. The present invention contemplates all such compounds, including cis- and trans-isomers, R- and S-enantiomers, diastereomers, (d)-isomers, (l)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this disclosure.

Isomeric mixtures containing any of a variety of isomer ratios may be utilized in accordance with the present disclosure. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present disclosure. Those of ordinary skill in the art will readily appreciate that analogous ratios are contemplated for more complex isomer mixtures.

The present disclosure also includes isotopically-labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorous, sulfur, fluorine, and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present disclosure, or an enantiomer, diastereomer, tautomer, or pharmaceutically acceptable salt or solvate thereof, which contain the aforementioned isotopes and/or other isotopes of other atoms are within the scope of this invention. Certain isotopically-labeled compounds of the present disclosure, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and hence, may be preferred in some circumstances. Isotopically labeled compounds can generally be prepared by carrying out the procedures disclosed in the Schemes and/or in the Examples below, by substituting a readily-available isotopically labeled reagent for a non-isotopically labeled reagent.

If, for instance, a particular enantiomer of a compound of the present disclosure is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic, substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, the term inhibitor of STAT3 as used herein refers to one or more molecules that interfere at least in part with the activity of STAT3 to perform one or more activities, including the ability of STAT3 to bind to a molecule and/or the ability to be phosphorylated.

As used herein, the term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

IR contributes to the genesis of complications in patients with CKD, but cellular mechanisms causing IR are not understood. One mechanism implicated is CKD-induced inflammation and the activation of the signal transducer and activator of transcription 3 (Stat3) in muscles of CKD mice is identified. Consequently, Stat3 activation increased the expression of Fbxo40, a muscle-specific E3 ubiquitin ligase that is involved in ubiquitinization and degradation of insulin receptor substrate 1 (IRS1) interrupting insulin signaling. Administration of a small molecule inhibitor of Stat3 (TTI101) to CKD or HFD mice results in significant improvements in glucose tolerance and insulin signaling in skeletal muscles. Muscle-specific Stat3 KO mice also developed improved glucose tolerance with HFD. The results indicate that Stat3 activation in muscles upregulates Fbxo40 leading to development of IR.

The present disclosure concerns methods, compounds, and compositions for treatment related to insulin resistance (IR) of any kind, including insulin resistance related to CKD.

### Methods of the Disclosure

Disclosed herein, in certain embodiments, are methods of treating insulin resistance in an individual in need thereof, including one at risk for insulin resistance. In some embodiments, the individual has insulin resistance as a result of an underlying condition. In some embodiments, the insulin resistance is associated with muscle of the individual. In some embodiments, the insulin resistance is caused by any reason for the individual, such as elevated free fatty acids in the blood, obesity, being overweight, having visceral fat, having a high fructose intake, having inflammation, being inactive, dysbiosis of the gut microbiota, and/or being genetically predisposed. An individual at risk for insulin resistance may be an individual that has elevated free fatty acids in the blood, has obesity, is overweight, has visceral fat, has a high fructose intake, having inflammation, being inactive, dysbiosis of the gut microbiota, and/or being genetically predisposed.

In some embodiments of the disclosure, the methods, compounds, and/or compositions of the disclosure are useful for treating and/or preventing insulin resistance and/or conditions related thereto, and in specific cases such treatment occurs by inhibiting Stat3 activity and/or expression. In some embodiments, compounds of the disclosure interact with the Stat3 SH2 domain, competitively inhibit recombinant Stat3 binding to its immobilized pY-peptide ligand, and/or inhibit IL-6-mediated tyrosine phosphorylation of Stat3, for example. In particular embodiments, the compounds and compositions of the disclosure fulfills the criteria of interaction analysis (CIA): 1) global minimum energy score ≤−30; 2) formation of a salt-bridge and/or H-bond network within the pY-residue binding site of Stat3; and/or 3) formation of a H-bond with or blocking access to the amide hydrogen of E638 of Stat3, for example. In some embodiments, the compound(s) and composition(s) interacts with a hydrophobic binding pocket with the Stat3 SH2 domain.

An underlying condition associated with insulin resistance may or may not be present and may or may not be known for the individual. An individual in need of therapy for insulin resistance may be an individual that has at least one symptom of insulin resistance or a condition associated thereto, or is susceptible to having insulin resistance or a condition associated thereto by having an underlying condition that can have insulin resistance as a condition or direct or indirect cause of insulin resistance.

Embodiments of the disclosure include methods for the treatment of insulin resistance in an individual known to have the insulin resistance, suspected of insulin resistance, or at risk for having insulin resistance. The compounds include small molecule STATS inhibitors and functional derivatives as described herein. In some embodiments, the individual is receiving an additional therapy for an underlying condition that is related to (and may be the direct or indirect cause of) the insulin resistance.

In some embodiments, the individual is known to have an underlying condition that often has insulin resistance as a precursor or as at least one symptom, and that individual may or may have not shown a sign of having insulin resistance. In cases wherein an individual has an underlying condition that often has insulin resistance as a precursor or as at least one symptom, the individual may be provided with an effective amount of one or more compounds or compositions of the disclosure prior to and/or after the appearance of insulin resistance. When the individual is provided one of more compounds or compositions prior to the appearance of insulin resistance, the onset of insulin resistance or an associated condition may be delayed or completely inhibited and/or the severity of the insulin resistance or an associated condition may be reduced, compared to the condition of the individual without having received the compound(s) or composition(s), for example.

An individual suspected of having insulin resistance (IR) or a condition associated therewith may or may not be subjected to diagnosis thereof as part of the method. An individual suspected of having insulin resistance may or may not be subject to determination that they have insulin resistance, such as through a blood test that checks blood sugar levels, for example. An individual may be clinically determined to have insulin resistance prior to subjecting them to methods of the disclosure, and such determination may include analysis of symptoms such as one or more of the following: (1) a waistline over 40 inches in men and 35 inches in women; (2) blood pressure readings of 130/80 or higher; (3) a fasting glucose level over 100 mg/dL; (4) a fasting triglyceride level over 150 mg/dL; (5) a HDL cholesterol level over under 40 mg/dL in men and 50 mg/dL in women; (6) skin tags; and (7) patches of dark, velvety skin called acanthosis nigricans. Therefore, an individual may be subjected to fasting plasma glucose test, oral glucose tolerance test, and/or hemoglobin A1c test as a determination that they have insulin resistance. If such tests respectively have the following outcomes, the individual may have insulin resistance: fasting plasma glucose test of 100-125; oral glucose tolerance test: 140-199 after a second test; A1c results of 5.7% to 6.4%.

Following such a determination, the individual may be subjected to methods encompassed by the disclosure.

In particular embodiments, methods of the disclosure reduce the risk or severity of medical conditions associated with insulin resistance or that are complications of insulin resistance at least in part, such as severe high blood sugar; severe low blood sugar; heart attack; stroke; kidney disease (including chronic, for example, chronic kidney disease (CKD)); eye problems; cancer; non-alcoholic fatty liver disease (NAFLD); polycystic ovarian syndrome (PCOS); metabolic syndrome; diabetes; or Alzheimer's disease, for example. The methods may allow for the prevention of such medical conditions that are associated with insulin resistance, including the delay of their onset, reduction of their severity, and/or allowing for more effective treatment of the conditions. Embodiments of the disclosure include methods that reverse insulin resistance and, by doing so, reduce the risk of having the associated medical conditions. Thus, in specific aspects, an individual is provided an effective amount of one or more STATS inhibitors and as a result reverses insulin resistance and treats or reduces the risk of having an associated medical condition.

In specific embodiments, the individual has chronic kidney disease (CKD) or is at risk thereof, compared to the general population, for example. CKD risk factors include having insulin resistance, diabetes, high blood pressure, heart disease, and/or a family history of kidney failure. CKD may be determined by a blood test that checks how well the kidneys are filtering the blood, called glomerular filtration rate (GFR). A GFR of less than 60 may indicate CKD. Another test for CKD includes a urine test to check for albumin that can pass into the urine when the kidneys are damaged, and a determination of more than 30 mg/g albumin indicates the presence of kidney damage.

In particular embodiments, insulin resistance is a hallmark of metabolic syndrome and type 2 diabetes, and the individual may be treated for insulin resistance with one or more STAT3 inhibitors that directly or indirectly provide treatment or prevention of metabolic syndrome or type 2 diabetes. Metabolic syndrome is a group of risk factors associated with type 2 diabetes and heart disease. Its symptoms include high blood triglycerides, blood pressure, belly fat, and blood sugar, as well as low HDL (good) cholesterol levels. Methods of being administered one or more STAT3 inhibitors allow for prevention of metabolic syndrome and type 2 diabetes by stopping the development of insulin resistance, in particular embodiments of the disclosure.

### Compounds

Disclosed herein, in certain embodiments, are methods of treating, preventing, and/or reducing the risk of insulin resistance or a condition associated thereto in an individual in need thereof, comprising administering one or more STAT3 inhibitor compounds disclosed herein.. Specific compounds are disclosed herein, but one of skill in the art recognizes that functional derivatives of such compounds are also encompassed by the disclosure. The term “derivative” as used herein is a compound that is formed from a similar compound or a compound that can be considered to arise from another compound, if one atom is replaced with another atom or group of atoms. Derivative can also refer to compounds that at least theoretically can be formed from the precursor compound. Derivatives of the compounds of the disclosure have the ability to inhibit STAT3 directly or indirectly, in particular embodiments.

In particular embodiments, the STAT3 inhibitor compound is selected from the group consisting of 4-[3-(2,3-dihydro-1,4-benzodioxin-6-yl)-3-oxo-1-propen-1-yl] benzoic acid; 4{5-[(3-ethyl-4-oxo-2-thioxo-1,3-thiazolidin-5-ylidene)methyl]-2-furyl}benzoic acid; 4-[({3-[(carboxymethyl)thio]-4-hydroxy-1-naphthyl}amino)sulfonyl] benzoic acid; 3-({2-chloro-4-[(1,3-dioxo-1,3-dihydro-2H-inden-2-ylidene)methyl]-6-ethoxyphenoxy}methyl)benzoic acid; methyl 4-({[3-(2-methyoxy-2-oxoethyl)-4,8-dimethyl-2-oxo-2H-chromen-7-yl]oxy}methyl)benzoate; 4-chloro-3 -{5-[(1,3 -diethyl-4,6-dioxo-2-thioxotetrahydro-5(2H)-pyrimidinylidene)methyl]-2-furyl}benzoic acid; a functionally active derivative thereof; and a mixture thereof.

In another embodiment, the STAT3 inhibitor compound is a compound according to Formula I:

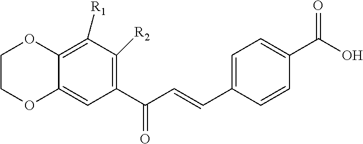

or a pharmaceutically acceptable salt thereof,
wherein R₁ and R₂ may be the same or different and are selected from the group consisting of hydrogen, carbon, sulfur, nitrogen, oxygen, fluorine, chlorine, bromine, iodine, alkanes, cyclic alkanes, alkane-based derivatives, alkenes, cyclic alkenes, alkene-based derivatives, alkynes, alkyne-based derivative, ketones, ketone-based derivatives, aldehydes, aldehyde-based derivatives, carboxylic acids, carboxylic acid-based derivatives, ethers, ether-based derivatives, esters and ester-based derivatives, amines, amino-based derivatives, amides, amide-based derivatives, monocyclic or polycyclic arene, heteroarenes. arene-based derivatives, heteroarene-based derivatives, phenols, phenol-based derivatives, benzoic acid, and benzoic acid-based derivatives.

In some embodiments, the STAT3 inhibitor compound is a compound of Formula II:

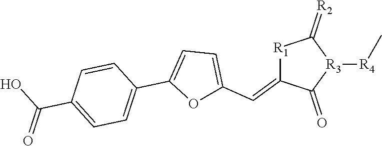

or a pharmaceutically acceptable salt thereof,
wherein R₁, and R₃ may be the same or different and are selected from the group consisting of hydrogen, carbon, nitrogen, sulfur, oxygen, fluorine, chlorine, bromine, iodine, alkanes. cyclic alkanes, alkane-based derivatives, alkenes, cyclic alkenes, alkene-based derivatives, alkynes, alkyne-based derivative, ketones, ketone-based derivatives, aldehydes, aldehyde-based derivatives, carboxylic acids, carboxylic acid-based derivatives, ethers, ether-based derivatives, esters and ester-based derivatives, amines, amino-based derivatives, amides, amide-based derivatives, monocyclic or polycyclic arene, heteroarenes. arene-based derivatives, heteroarene-based derivatives, phenols, phenol-based derivatives, benzoic acid, and benzoic acid-based derivatives; and R₂ and R₄ may be the same or different and are selected from the group consisting of hydrogen, alkanes,. cyclic alkanes, alkane-based derivatives, alkenes, cyclic alkenes, alkene-based derivatives, alkynes, alkyne-based derivative, ketones, ketone-based derivatives, aldehydes, aldehyde-based derivatives, carboxylic acids, carboxylic acid-based derivatives, ethers, ether-based derivatives, esters and ester-based derivatives, amines, amino-based derivatives, amides, amide-based derivatives, monocyclic or polycyclic arene, heteroarenes. arene-based derivatives, heteroarene-based derivatives, phenols, phenol-based derivatives, benzoic acid, and benzoic acid-based derivatives.

In some embodiments, the STATS inhibitor compound is a compound of Formula III:

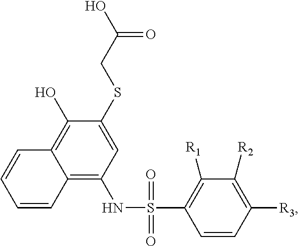

or a pharmaceutically acceptable salt thereof,
wherein R₁, R₂, and R₃ may be the same or different and are selected from the group consisting of hydrogen, carbon, nitrogen, sulfur, oxygen, fluorine, chlorine, bromine, iodine, carboxyl, alkanes. cyclic alkanes, alkane-based derivatives, alkenes, cyclic alkenes, alkene-based derivatives, alkynes, alkyne-based derivative, ketones, ketone-based derivatives, aldehydes, aldehyde-based derivatives, carboxylic acids, carboxylic acid-based derivatives, ethers, ether-based derivatives, esters and ester-based derivatives, amines, amino-based derivatives, amides, amide-based derivatives, monocyclic or polycyclic arene, heteroarenes. arene-based derivatives, heteroarene-based derivatives, phenols, phenol-based derivatives, benzoic acid, and benzoic acid-based derivatives.

In some embodiments, the STAT3 inhibitor compound is selected from the group consisting of N-(1′,2-dihydroxy-1,2′-binaphthalen-4′-yl)-4-methoxybenzenesulfonamide, N-(3,1′-Dihydroxy-[1,2]binaphthalenyl-4′-yl)-4-methoxy-benzenesulfonamide, N-(4,1′-Dihydroxy-[1,2]binaphthalenyl-4′-yl)-4-methoxy-benzenesulfonamide, N-(5,1′-Dihydroxy-[1,2]binaphthalenyl-4′-yl)-4-methoxy-benzenesulfonamide, N-(6,1′-Dihydroxy-[1,2]binaphthalenyl-4′-yl)-4-methoxy-benzenesulfonamide, N-(7,1′-Dihydroxy-[1,2]binaphthalenyl-4′-yl)-4-methoxy-benzenesulfonamide, N-(8,1′-Dihydroxy-[1,2]binaphthalenyl-4′-yl)-4-methoxy-benzenesulfonamide, 4-Bromo-N-(1,6′-dihydroxy-[2,2]binaphthalenyl-4-yl)-benzenesulfonamide, and 4-Bromo-N-[4-hydroxy-3-(1H-[1,2,4]triazol-3-ylsulfanyl)-naphthalen-1-yl]-benzenesulfonamide, or a pharmaceutically acceptable salt thereof.

In some embodiments, the STAT3 inhibitor compound is N-(1′,2-dihydroxy-1,2′-binaphthalen-4′-yl)-4-methoxybenzenesulfonamide, or a pharmaceutically acceptable salt thereof. TTI-101 as used in the Example refers to N-(1′,2-dihydroxy-1,2′-binaphthalen-4′-yl)-4-methoxybenzenesulfonamide.

In some embodiments, the STAT3 inhibitor compound is a compound of Formula IV,

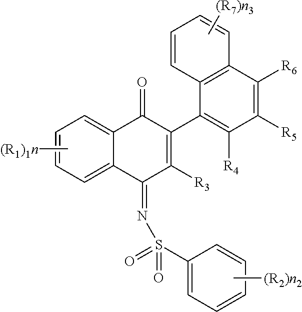

or a pharmaceutically acceptable salt thereof is described, wherein
each occurrence of R₁ is independently hydrogen, halogen, cyano, nitro, CF₃, OCF₃, ORₐ, SRₐ, C(═O)Rₐ, OC(═O)Rₐ, C(═O)ORₐ, NR_{b}R_{c}, NR_{b}C(═O)R_{c}, C(═O)NR_{b}R_{c}, NR_{b}C(═O)OR_{c}, OC(═O)NR_{b}R_{c}, NRₐC(═O)NR_{b}R_{c}, alkyl, alkenyl, cycloalkyl, optionally substituted aryl, or optionally substituted heterocycle;
m is 0, 1, 2, 3, or 4;
each occurrence of R₂ is independently hydrogen, halogen, cyano, nitro, CF₃, OCF₃, ORₐ, SRₐ, C(═O)Rₐ, OC(═O)Rₐ, C(═O)ORₐ, NR_{b}R_{c}, NR_{b}C(═O)R_{c}, C(═O)NR_{b}R_{c}, NR_{b}C(═O)OR_{c}, OC(═O)NR_{b}R_{c}, NRₐC(═O)NR_{b}R_{c}, alkyl, alkenyl, cycloalkyl, cycloalkenyl, optionally substituted aryl, optionally substituted aryloxyl, or optionally substituted heterocycle;
n2 is 0, 1, 2, 3, 4, or 5;
R₃ is hydrogen, halogen, cyano, nitro, CF₃, OCF₃, ORₐ, SRₐ, OC(═O)Rₐ, alkyl, alkenyl, cycloalkyl, or optionally substituted aryl or heteroaryl;
R₄ is hydrogen, halogen, cyano, nitro, CF₃, OCF₃, ORₐ, SRₐ, NR_{b}R_{c}, OC(═O)Rₐ, alkyl, alkenyl, or cycloalkyl;
each occurrence of R₅, R₆, and R₇ is independently hydrogen, halogen, cyano, nitro, CF₃, OCF₃, ORₐ, SRₐ, C(═O)Rₐ, OC(═O)Rₐ, C(═O)ORₐ, NR_{b}R_{c}, NR_{b}C(═O)R_{c}, C(═O)NR_{b}R_{c}, NR_{b}C(═O)OR_{c}, OC(═O)NR_{b}R_{c}, NRₐC(═O)NR_{b}R_{c}, alkyl, alkenyl, cycloalkyl, optionally substituted aryl, or optionally substituted heterocycle;
n3 is 0, 1, 2, 3, or 4; and
each occurrence of Rₐ, R_{b}, and R_{c} is independently hydrogen, alkyl, cycloalkyl, alkenyl, cycloalkenyl, alkynyl, heterocycle, or aryl; or said R_{b} and R_{c} together with the nitrogen atom to which they are bonded optionally form a heterocycle comprising 1-4 heteroatoms.

In any one or more of the embodiments for Formula IV, each occurrence of R₁ is independently hydrogen, halogen, cyano, nitro, CF₃, OCF₃, ORₐ, or SRₐ.

In alternative embodiments for Formula IV, each occurrence of R₁ is independently C(═O)Rₐ, OC(═O)Rₐ, C(═O)ORₐ, NRₐR_{b}, NR_{b}C(═O)Rₐ, C(═O)NR_{b}R_{c}, NR_{b}C(═O)ORₐ, OC(═O)NR_{b}R_{c}, or NRₐC(═O)NR_{b}R_{c}.

In any one or more of the embodiments for Formula IV, each occurrence of R₁ is independently alkyl, alkenyl, cycloalkyl, optionally substituted aryl, or optionally substituted heterocycle.

In any one or more of the embodiments for Formula IV, R₁ is H.

In any one or more of the embodiments for Formula IV, n₁ is 0, 1, or 2.

In any one or more of the embodiments for Formula IV, n1 is 1.

In any one or more of the embodiments for Formula IV, n₁ is 0.

In any one or more of the embodiments for Formula IV, each occurrence of R₂ is independently hydrogen, halogen, cyano, nitro, CF₃, OCF₃, ORₐ, or SRₐ.

In alternative embodiments for Formula IV, each occurrence of R₂ is independently C(═O)Rₐ, OC(═O)Rₐ, C(═O)ORₐ, NRₐR_{b}, NR_{b}C(═O)Rₐ, C(═O)NR_{b}R_{c}, NR_{b}C(═O)ORₐ, OC(═O)NR_{b}R_{c}, or NRₐC(═O)NR_{b}R_{c}.

In any one or more of the embodiments for Formula IV, each occurrence of R₂ is independently alkyl, alkenyl, cycloalkyl, optionally substituted aryl, or optionally substituted heterocycle.

In any one or more of the embodiments for Formula IV, R₂ is H.

In any one or more of the embodiments for Formula IV, n₂ is 0, 1, or 2.

In any one or more of the embodiments for Formula IV, n₂ is 1.

In any one or more of the embodiments for Formula IV, n₂ is 0.

In any one or more of the embodiments for Formula IV, R₃ is hydrogen, halogen, cyano, nitro, or CF₃.

In any one or more of the embodiments for Formula IV, R₃ is OCF₃, ORₐ, SRₐ, or OC(═O)Rₐ.

In any one or more of the embodiments for Formula IV, R₃ is alkyl, alkenyl, or cycloalkyl.

In any one or more of the embodiments for Formula IV, R₃ is H.

In any one or more of the embodiments for Formula IV, R₄ is hydrogen, halogen, cyano, nitro, or ORₐ.

In any one or more of the embodiments for Formula IV, R₄ is OCF₃, SRₐ, or OC(═O)Rₐ.

In any one or more of the embodiments for Formula IV, R₄ is alkyl, alkenyl, or cycloalkyl.

In alternative embodiments of compounds for Formula IV, R₄ is OH.

In alternative embodiments of compounds for Formula IV, R₄ is OMe.

In any one or more of the embodiments for Formula IV, R₅, R₆, and R₇ are each independently selected from the group consisting of hydrogen, halogen, cyano, nitro, and CF₃.

In any one or more of the embodiments for Formula IV, R₅, R₆, and R₇ are each independently selected from the group consisting of OCF₃, ORₐ, and SRₐ.

In any one or more of the embodiments for Formula IV, R₅, R₆, and R₇ are each independently selected from the group consisting of OCF₃ and ORₐ.

In alternative embodiments of compounds for Formula IV, R₅, R₆, and R₇ are each independently selected from the group consisting of C(═O)Rₐ, OC(═O)Rₐ, C(═O)ORₐ, NRₐR_{b}, NR_{b}C(═O)Rₐ, C(═O)NR_{b}R_{c}, NR_{b}C(═O)ORₐ, OC(═O)NR_{b}R_{c}, and NRₐC(═O)NR_{b}R_{c}.

In any one or more of the embodiments for Formula IV, R₅, R₆, and R₇ are each independently selected from the group consisting of alkyl, alkenyl, cycloalkyl, optionally substituted aryl, and optionally substituted heterocycle.

In any one or more of the embodiments for Formula IV, each occurrence of R₅, R₆, and R₇ is H.

In any one or more of the embodiments for Formula IV, n₃ is 0, 1, or 2.

In any one or more of the embodiments for Formula IV, n₃ is 1.

In any one or more of the embodiments for Formula IV, n₃ is 0.

In any one or more of the embodiments for Formula IV, each occurrence of Rₐ is independently hydrogen, alkyl, heterocycle, or aryl.

In any one or more of the embodiments for Formula IV, each occurrence of Rₐ is independently hydrogen or alkyl.

In any one or more of the embodiments for Formula IV, each occurrence of R_{b} and R_{c} is independently hydrogen, alkyl, heterocycle, or aryl.

In any one or more of the embodiments for Formula IV, each occurrence of R_{b} and R_{c} is independently hydrogen or alkyl.

In any one or more of the embodiments for Formula IV, R_{b} and R_{c} together with the nitrogen atom to which they are bonded optionally form a heterocycle comprising 1-4 heteroatoms each selected from the group consisting of N, O, and S.

In any one or more of the embodiments described herein, the STAT3 inhibitor compound has the structure of Formula V:

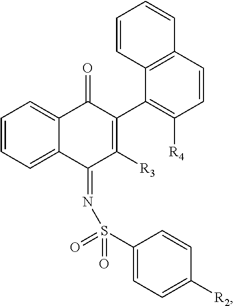

or a pharmaceutically acceptable salt thereof.

In any one or more of the embodiments for Formula V, R₂ is H, OH, alkyl, alkoxy, halogen, NR_{b}R_{c}, CF₃, OCF₃, or CN.

In any one or more of the embodiments for Formula V, R₂ is NH₂, OH, OMe, OEt, OCH₂CH₂CH₃, or OCH(CH₃)₂.

In any one or more of the embodiments for Formula V, R₂ is selected from the group consisting of hydrogen, methyl, ethyl, propyl, tert-butyl, F, Cl, Br, CF₃, nitro, methoxy, ethoxy, OCF₃, —C(═O)Me, —C(═O)OMe, —NHC(═O)Me, 1,4-dioxanyl, cyclohexanyl, cyclohexenyl, phenoxy, 2-methoxyphenoxy, 3-methoxyphenoxy, 4-methoxyphenoxy, 2-chlorophenoxy, 3-chlorophenoxy, 4-chlorophenoxy, 2-methylphenoxy, 3-methylphenoxy, and 4-methylphenoxy.

In any one or more of the embodiments for Formula V, R₂ is OMe.

In any one or more of the embodiments for Formula V, R₃ is H, OH, alkyl, alkoxy, or halogen.

In any one or more of the embodiments for Formula V, R₃ is H.

In any one or more of the embodiments for Formula V, R₄ is H, alkyl, OH, NH₂, alkoxy, halogen, CF₃, or CN.

In any one or more of the embodiments for Formula V, R₄ is H, OH, or alkoxy.

In any one or more of the embodiments for Formula V, R₄ is OH.

In any one or more of the embodiments for Formula V, R₄ is OMe.

In any one or more of the embodiments described herein, the STAT3 inhibitor compound has the structure of Formula VI,

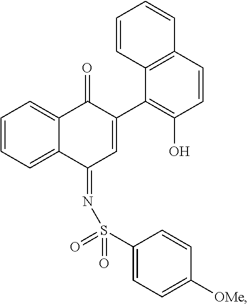 [SMILES: COc1ccc(S(=O)(=O)/N=C2\C=C(c3c(O)ccc4ccccc34)C(=O)c3ccccc32)cc1]

or a pharmaceutically-acceptable salt thereof.

In any one or more of the embodiments described herein, the compound is selected from the group consisting of the compounds in Table 1a, or a pharmaceutically acceptable salt thereof.

In any one or more of the embodiments described herein, the STAT3 inhibitor compound is selected from the group consisting of the compounds in Table lb, or a pharmaceutically-acceptable salt thereof.

In some embodiments, the compound of Formula IV is selected from the Examples of compounds shown in Table 1a, or a pharmaceutically acceptable salt thereof. The enumerated compounds in Table 1a are representative and non-limiting examples of compounds of Formula IV.

TABLE 1a
Selected compounds of Formula IV, where n₁, n₂, and n₃ are independently 1 or 2.

| Ex- | | | | | | | |
|---|---|---|---|---|---|---|---|
| am- | | | | | | | |
| ple | | | | | | | |
| No. | R₁ | R₂ | R₃ | R₄ | R₅ | R₆ | R₇ |
| 100 | H | Cl | H | H | H | F | F |
| 102 | F | H | F | H | F | OH | Cl |
| 103 | Cl | 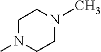 | Cl | OH | Cl | OH | NO₂ |
| 104 | CN | OH | CN | OH | Br | OMe | OCF₃ |
| 105 | NO₂ | OMe | CF₃ | OH | NO₂ | NH₂ | SH |
| 106 | CF₃ | OEt | Me | OMe | OCF₃ | NH₂ | SH |
| 107 | OCF₃ | OPr | Et | NH₂ | OCF₃ | SCH₃ | OH |
| 108 | OH | OBu | Pr | NH₂ | SH | COOH | CONH₂ |
| 109 | OH | NH₂ | Bu | NH₂ | SH | COOH | CONH₂ |
| 110 | SH | SH | Cyclopropyl | SH | OH | Ph | OH |
| 111 | COOH | Me | H | SCH₃ | OH | pyridinyl | OH |
| 112 | COOMe | CONH₂ | H | COOH | CONH₂ | Ph | OH |
| 113 | CONH₂ | NH(C═O)Me | —CH═CH₂ | COOH | CONH₂ | Me | H |
| 114 | CONMe₂ | cyclopropyl | Ph | OH | CONH₂ | Et | H |
| 115 | NH(C═O)Me | Ph | pyridinyl | OH | NH(C═O)Me | Pr | Me |
| 116 | Me | 3-fluorophenyl | Me | OH | Me | cyclobutyl | Et |
| 117 | Ph | 4-pyridinyl | H | NHMe | Et | CF₃ | cyclopropyl |
| 118 | 4-chlorophenyl | NO₂ | H | NHMe | cyclobutyl | OCF₃ | NO₂ |
| 119 | 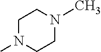 | CF₃ | H | NMe₂ | 4-chlorophenyl | SH | pyridinyl |
| 120 | cyclobutyl | OH | H | 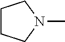 | Ph | Ph | 4-pyridinyl |
| 121 | 4-pyridinyl | SH | H | 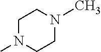 | OEt | 4-chlorophenyl | CONMe₂ |
| 122 | OEt | Me | Me | Me | OPr | OPr | NH(C═O)Me |
| 123 | OPr | Et | Ph | Me | 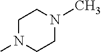 | 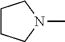 | 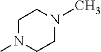 |
| 124 | OBu | Pr | Ph | Et | 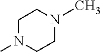 | 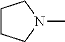 | 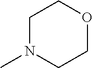 |

In some embodiments, the compound of Formula V is selected from the Examples of compounds shown in Table lb, or a pharmaceutically acceptable salt thereof. The enumerated compounds in Table lb are representative and non-limiting examples of compounds of Formula V.

TABLE 1b
Selected compounds of Formula V.

| Ex- | | | |
|---|---|---|---|
| am- | | | |
| ple | | | |
| No. | R₂ | R₃ | R₄ |
| 125 | Cl | H | H |
| 126 | H | F | OH |
| 127 | 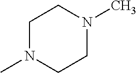 | Cl | OH |
| 128 | OH | H | OH |
| 129 | OMe | CF₃ | OH |
| 130 | OEt | Me | OMe |
| 131 | OPr | Et | NH₂ |
| 132 | OBu | Pr | NH₂ |
| 133 | NH₂ | Bu | NH₂ |
| 134 | SH | cyclopropyl | SH |
| 135 | Me | H | SCH₃ |
| 136 | CONH₂ | H | COOH |
| 137 | NH(C═O)Me | —CH═CH₂ | COOH |
| 138 | cyclopropyl | Ph | OH |
| 139 | Ph | pyridinyl | OH |
| 140 | 3-fluorophenyl | Me | OH |
| 141 | 4-pyridinyl | H | NHMe |
| 142 | NO₂ | H | NHMe |
| 143 | CF₃ | H | NMe₂ |
| 144 | OH | H | 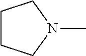 |
| 145 | SH | H | 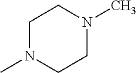 |
| 146 | Me | Me | Me |
| 147 | Et | Ph | Me |
| 148 | Pr | Ph | Et |

Stat3 inhibitors contemplated in this invention include compounds with structures within any one of the following tables:

TABLE 2

| Structure | Formula structure |
|---|---|
| 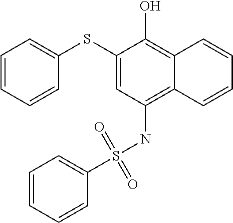 [SMILES: O=S(=O)(Nc1cc(Sc2ccccc2)c(O)c2ccccc12)c1ccccc1] | C22H17NO3S2 |
| 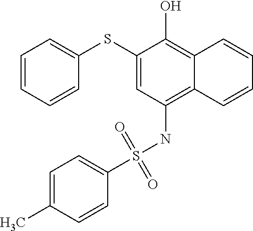 [SMILES: Cc1ccc(S(=O)(=O)Nc2cc(Sc3ccccc3)c(O)c3ccccc23)cc1] | C23H19NO3S2 |
| 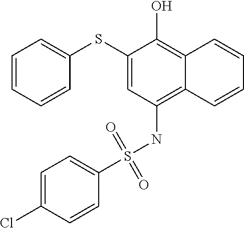 [SMILES: O=S(=O)(Nc1cc(Sc2ccccc2)c(O)c2ccccc12)c1ccc(Cl)cc1] | C22H16ClNO3S2 |
| 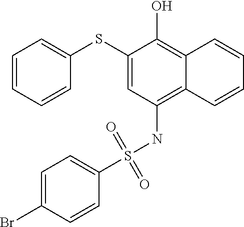 [SMILES: O=S(=O)(Nc1cc(Sc2ccccc2)c(O)c2ccccc12)c1ccc(Br)cc1] | C22H16BrNO3S2 |
| 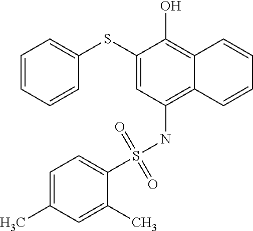 [SMILES: Cc1ccc(S(=O)(=O)Nc2cc(Sc3ccccc3)c(O)c3ccccc23)c(C)c1] | C24H21NO3S2 |
| 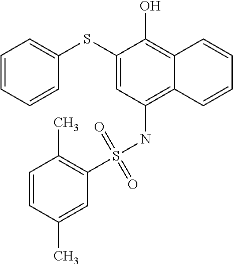 [SMILES: Cc1ccc(C)c(S(=O)(=O)Nc2cc(Sc3ccccc3)c(O)c3ccccc23)c1] | C24H21NO3S2 |
| 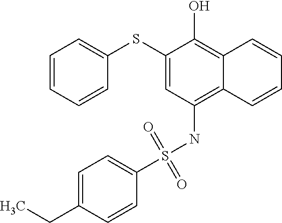 [SMILES: CCc1ccc(S(=O)(=O)Nc2cc(Sc3ccccc3)c(O)c3ccccc23)cc1] | C24H21NO3S2 |
| 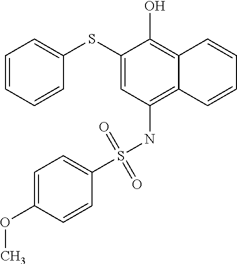 [SMILES: COc1ccc(S(=O)(=O)Nc2cc(Sc3ccccc3)c(O)c3ccccc23)cc1] | C23H19NO4S2 |
| 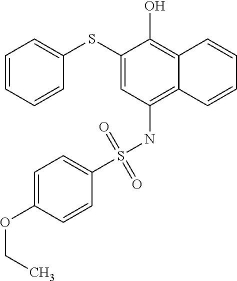 [SMILES: CCOc1ccc(S(=O)(=O)Nc2cc(Sc3ccccc3)c(O)c3ccccc23)cc1] | C24H21NO4S2 |
| 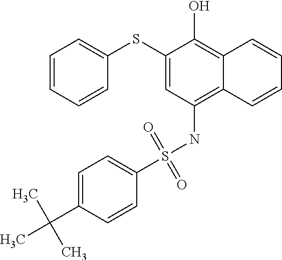 [SMILES: CC(C)(C)c1ccc(S(=O)(=O)Nc2cc(Sc3ccccc3)c(O)c3ccccc23)cc1] | C26H25NO3S2 |
| 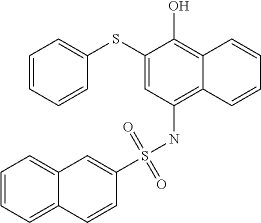 [SMILES: O=S(=O)(Nc1cc(Sc2ccccc2)c(O)c2ccccc12)c1ccc2ccccc2c1] | C26H19NO3S2 |
| 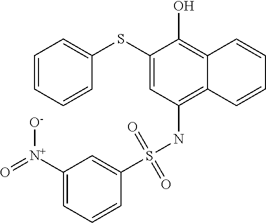 [SMILES: O=[N+]([O-])c1cccc(S(=O)(=O)Nc2cc(Sc3ccccc3)c(O)c3ccccc23)c1] | C22H16N2O5S2 |
| 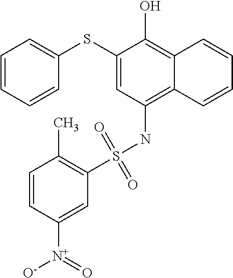 [SMILES: Cc1ccc([N+](=O)[O-])cc1S(=O)(=O)Nc1cc(Sc2ccccc2)c(O)c2ccccc12] | C23H18N2O5S2 |
| 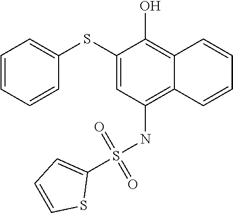 [SMILES: O=S(=O)(Nc1cc(Sc2ccccc2)c(O)c2ccccc12)c1cccs1] | C20H15NO3S3 |
| 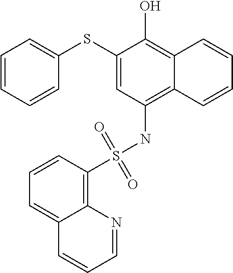 [SMILES: O=S(=O)(Nc1cc(Sc2ccccc2)c(O)c2ccccc12)c1cccc2cccnc12] | C25H18N2O3S2 |
| 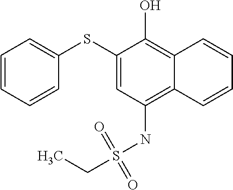 [SMILES: CCS(=O)(=O)Nc1cc(Sc2ccccc2)c(O)c2ccccc12] | C18H17NO3S2 |
| 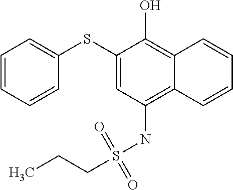 [SMILES: CCCS(=O)(=O)Nc1cc(Sc2ccccc2)c(O)c2ccccc12] | C19H19NO3S2 |
| 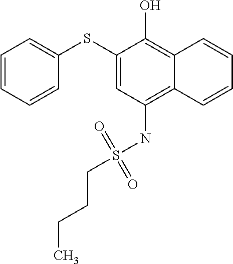 [SMILES: CCCCS(=O)(=O)Nc1cc(Sc2ccccc2)c(O)c2ccccc12] | C20H21NO3S2 |
| 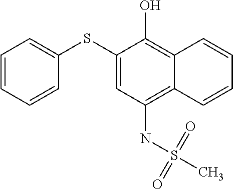 [SMILES: CS(=O)(=O)Nc1cc(Sc2ccccc2)c(O)c2ccccc12] | C17H15NO3S2 |
| 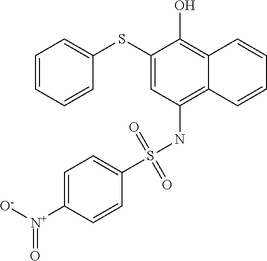 [SMILES: O=[N+]([O-])c1ccc(S(=O)(=O)Nc2cc(Sc3ccccc3)c(O)c3ccccc23)cc1] | C22H16N2O5S2 |
| 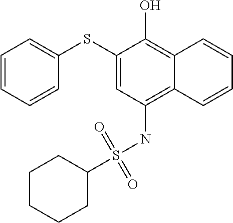 [SMILES: O=S(=O)(Nc1cc(Sc2ccccc2)c(O)c2ccccc12)C1CCCCC1] | C22H23NO3S2 |
| 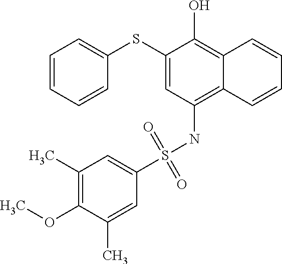 [SMILES: COc1c(C)cc(S(=O)(=O)Nc2cc(Sc3ccccc3)c(O)c3ccccc23)cc1C] | C25H23NO4S2 |
| 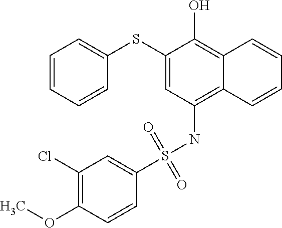 [SMILES: COc1ccc(S(=O)(=O)Nc2cc(Sc3ccccc3)c(O)c3ccccc23)cc1Cl] | C23H18ClNO4S2 |
| 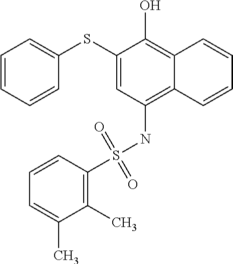 [SMILES: Cc1cccc(S(=O)(=O)Nc2cc(Sc3ccccc3)c(O)c3ccccc23)c1C] | C24H21NO3S2 |
| 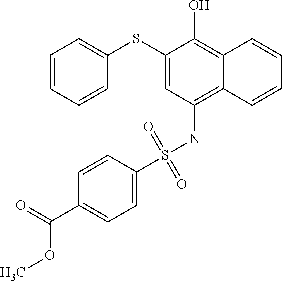 [SMILES: COC(=O)c1ccc(S(=O)(=O)Nc2cc(Sc3ccccc3)c(O)c3ccccc23)cc1] | C24H19NO5S2 |
| 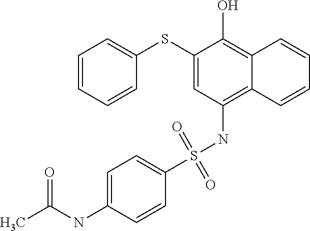 [SMILES: CC(=O)Nc1ccc(S(=O)(=O)Nc2cc(Sc3ccccc3)c(O)c3ccccc23)cc1] | C24H20N2O4S2 |
| 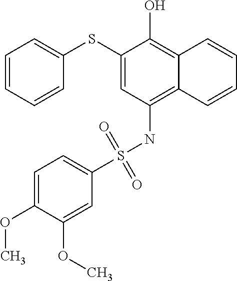 [SMILES: COc1ccc(S(=O)(=O)Nc2cc(Sc3ccccc3)c(O)c3ccccc23)cc1OC] | C24H21NO5S2 |
| 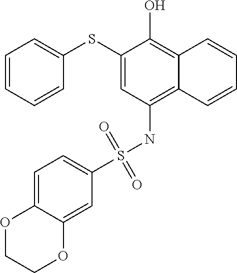 [SMILES: O=S(=O)(Nc1cc(Sc2ccccc2)c(O)c2ccccc12)c1ccc2c(c1)OCCO2] | C24H19NO5S2 |
| 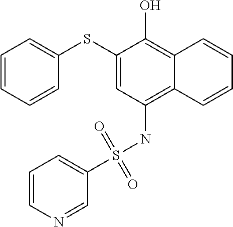 [SMILES: O=S(=O)(Nc1cc(Sc2ccccc2)c(O)c2ccccc12)c1cccnc1] | C21H16N2O3S2 |
| 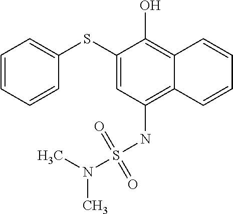 [SMILES: CN(C)S(=O)(=O)Nc1cc(Sc2ccccc2)c(O)c2ccccc12] | C18H18N2O3S2 |
| 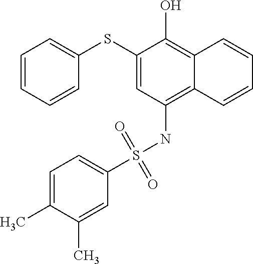 [SMILES: Cc1ccc(S(=O)(=O)Nc2cc(Sc3ccccc3)c(O)c3ccccc23)cc1C] | C24H21NO3S2 |
| 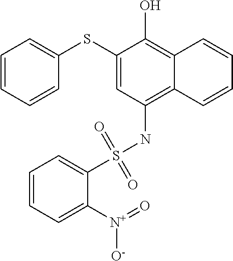 [SMILES: O=[N+]([O-])c1ccccc1S(=O)(=O)Nc1cc(Sc2ccccc2)c(O)c2ccccc12] | C22H16N2O5S2 |
| 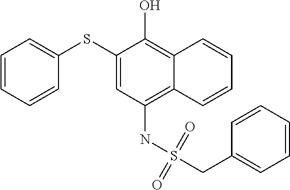 [SMILES: O=S(=O)(Cc1ccccc1)Nc1cc(Sc2ccccc2)c(O)c2ccccc12] | C23H19NO3S2 |
| 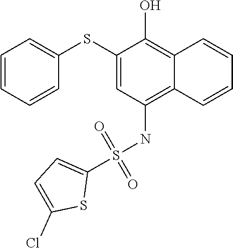 [SMILES: O=S(=O)(Nc1cc(Sc2ccccc2)c(O)c2ccccc12)c1ccc(Cl)s1] | C20H14ClNO3S3 |
| 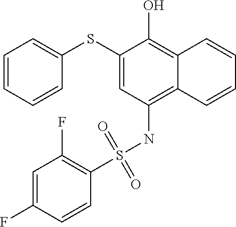 [SMILES: O=S(=O)(Nc1cc(Sc2ccccc2)c(O)c2ccccc12)c1ccc(F)cc1F] | C22H15F2NO3S2 |
| 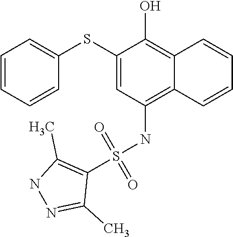 [SMILES: Cc1n[nH]c(C)c1S(=O)(=O)Nc1cc(Sc2ccccc2)c(O)c2ccccc12] | C21H19N3O3S2 |
| 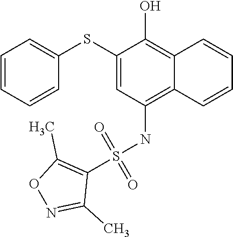 [SMILES: Cc1noc(C)c1S(=O)(=O)Nc1cc(Sc2ccccc2)c(O)c2ccccc12] | C21H18N2O4S2 |
| 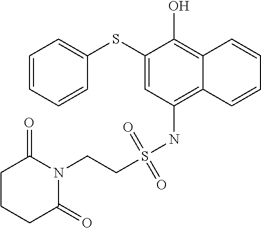 [SMILES: O=C1CCCC(=O)N1CCS(=O)(=O)Nc1cc(Sc2ccccc2)c(O)c2ccccc12] | C23H22N2O5S2 |
| 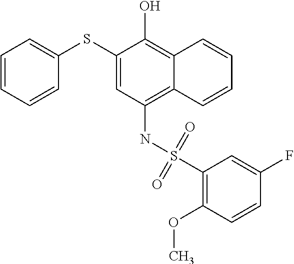 [SMILES: COc1ccc(F)cc1S(=O)(=O)Nc1cc(Sc2ccccc2)c(O)c2ccccc12] | C23H18FNO4S2 |
| 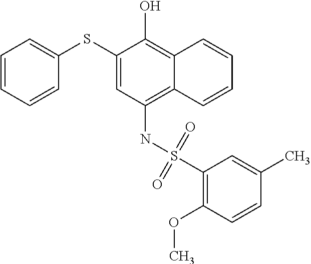 [SMILES: COc1ccc(C)cc1S(=O)(=O)Nc1cc(Sc2ccccc2)c(O)c2ccccc12] | C24H21NO4S2 |
| 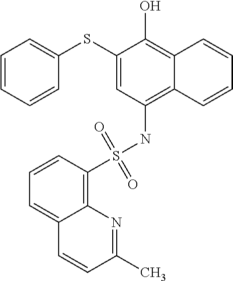 [SMILES: Cc1ccc2cccc(S(=O)(=O)Nc3cc(Sc4ccccc4)c(O)c4ccccc34)c2n1] | C26H20N2O3S2 |
| 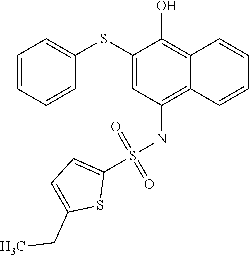 [SMILES: CCc1ccc(S(=O)(=O)Nc2cc(Sc3ccccc3)c(O)c3ccccc23)s1] | C22H19NO3S3 |
| 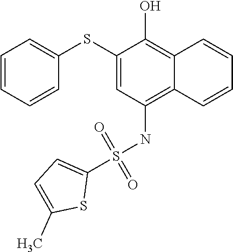 [SMILES: Cc1ccc(S(=O)(=O)Nc2cc(Sc3ccccc3)c(O)c3ccccc23)s1] | C21H17NO3S3 |
| 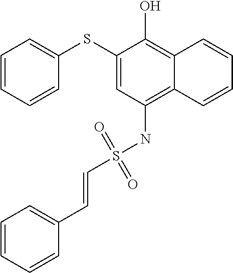 [SMILES: O=S(=O)(/C=C/c1ccccc1)Nc1cc(Sc2ccccc2)c(O)c2ccccc12] | C24H19NO3S2 |
| 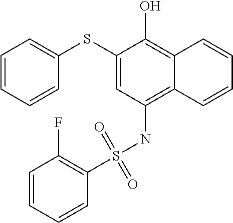 [SMILES: O=S(=O)(Nc1cc(Sc2ccccc2)c(O)c2ccccc12)c1ccccc1F] | C22H16FNO3S2 |
| 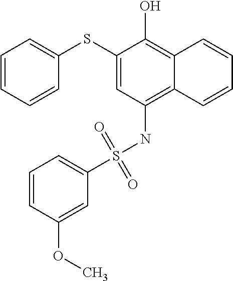 [SMILES: COc1cccc(S(=O)(=O)Nc2cc(Sc3ccccc3)c(O)c3ccccc23)c1] | C23H19NO4S2 |
| 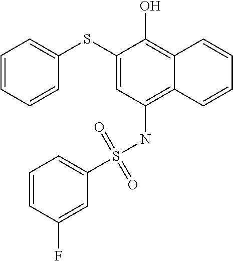 [SMILES: O=S(=O)(Nc1cc(Sc2ccccc2)c(O)c2ccccc12)c1cccc(F)c1] | C22H16FNO3S2 |
| 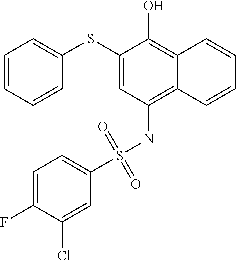 [SMILES: O=S(=O)(Nc1cc(Sc2ccccc2)c(O)c2ccccc12)c1ccc(F)c(Cl)c1] | C22H15ClFNO3S2 |
| 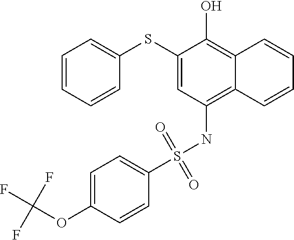 [SMILES: O=S(=O)(Nc1cc(Sc2ccccc2)c(O)c2ccccc12)c1ccc(OC(F)(F)F)cc1] | C23H16F3NO4S2 |
| 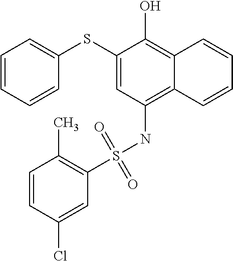 [SMILES: Cc1ccc(Cl)cc1S(=O)(=O)Nc1cc(Sc2ccccc2)c(O)c2ccccc12] | C23H18ClNO3S2 |
| 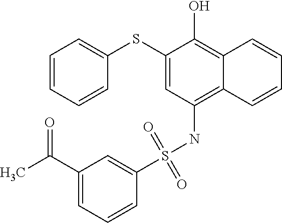 [SMILES: CC(=O)c1cccc(S(=O)(=O)Nc2cc(Sc3ccccc3)c(O)c3ccccc23)c1] | C24H19NO4S2 |
| 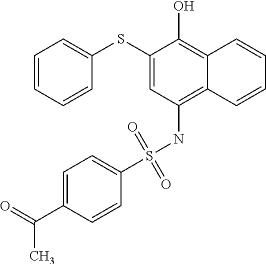 [SMILES: CC(=O)c1ccc(S(=O)(=O)Nc2cc(Sc3ccccc3)c(O)c3ccccc23)cc1] | C24H19NO4S2 |
| 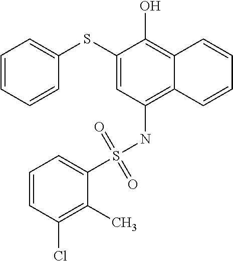 [SMILES: Cc1c(Cl)cccc1S(=O)(=O)Nc1cc(Sc2ccccc2)c(O)c2ccccc12] | C23H18ClNO3S2 |
| 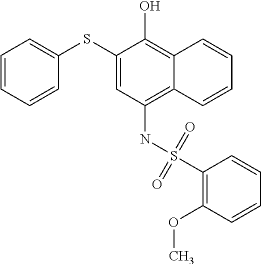 [SMILES: COc1ccccc1S(=O)(=O)Nc1cc(Sc2ccccc2)c(O)c2ccccc12] | C23H19NO4S2 |
| 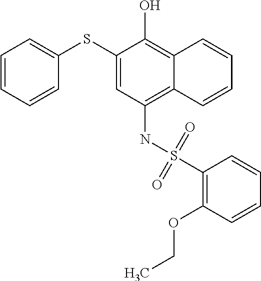 [SMILES: CCOc1ccccc1S(=O)(=O)Nc1cc(Sc2ccccc2)c(O)c2ccccc12] | C24H21NO4S2 |
| 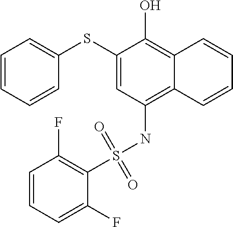 [SMILES: O=S(=O)(Nc1cc(Sc2ccccc2)c(O)c2ccccc12)c1c(F)cccc1F] | C22H15F2NO3S2 |
| 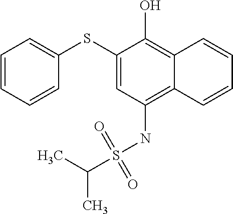 [SMILES: CC(C)S(=O)(=O)Nc1cc(Sc2ccccc2)c(O)c2ccccc12] | C19H19NO3S2 |
| 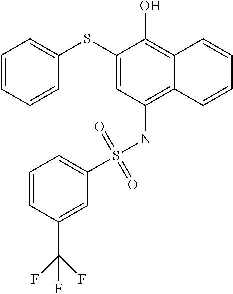 [SMILES: O=S(=O)(Nc1cc(Sc2ccccc2)c(O)c2ccccc12)c1cccc(C(F)(F)F)c1] | C23H16F3NO3S2 |
| 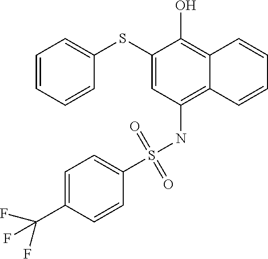 [SMILES: O=S(=O)(Nc1cc(Sc2ccccc2)c(O)c2ccccc12)c1ccc(C(F)(F)F)cc1] | C23H16F3NO3S2 |
| 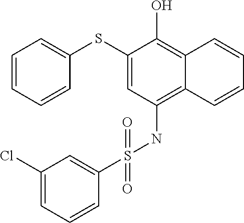 [SMILES: O=S(=O)(Nc1cc(Sc2ccccc2)c(O)c2ccccc12)c1cccc(Cl)c1] | C22H16ClNO3S2 |
| 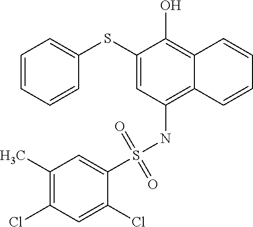 [SMILES: Cc1cc(S(=O)(=O)Nc2cc(Sc3ccccc3)c(O)c3ccccc23)c(Cl)cc1Cl] | C23H17Cl2NO3S2 |
| 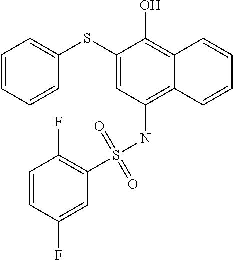 [SMILES: O=S(=O)(Nc1cc(Sc2ccccc2)c(O)c2ccccc12)c1cc(F)ccc1F] | C22H15F2NO3S2 |
| 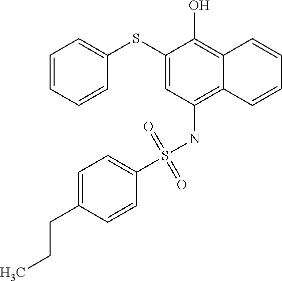 [SMILES: CCCc1ccc(S(=O)(=O)Nc2cc(Sc3ccccc3)c(O)c3ccccc23)cc1] | C25H23NO3S2 |
| 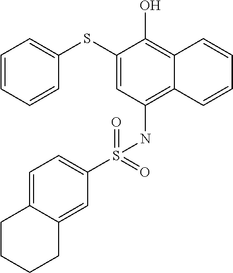 [SMILES: O=S(=O)(Nc1cc(Sc2ccccc2)c(O)c2ccccc12)c1ccc2c(c1)CCCC2] | C26H23NO3S2 |
| 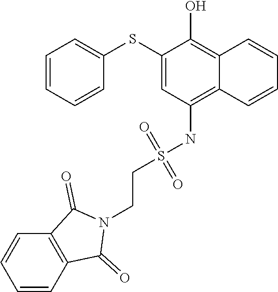 [SMILES: O=C1c2ccccc2C(=O)N1CCS(=O)(=O)Nc1cc(Sc2ccccc2)c(O)c2ccccc12] | C26H20N2O5S2 |
| 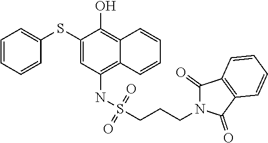 [SMILES: O=C1c2ccccc2C(=O)N1CCCS(=O)(=O)Nc1cc(Sc2ccccc2)c(O)c2ccccc12] | C27H22N2O5S2 |
| 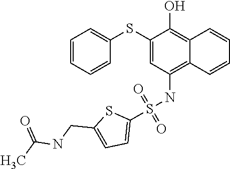 [SMILES: CC(=O)NCc1ccc(S(=O)(=O)Nc2cc(Sc3ccccc3)c(O)c3ccccc23)s1] | C23H20N2O4S3 |
| 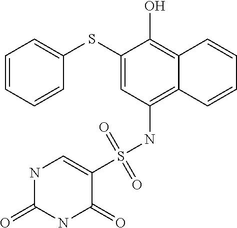 [SMILES: O=c1[nH]cc(S(=O)(=O)Nc2cc(Sc3ccccc3)c(O)c3ccccc23)c(=O)[nH]1] | C20H15N3O5S2 |
| 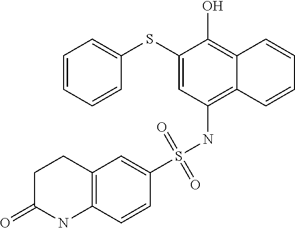 [SMILES: O=C1CCc2cc(S(=O)(=O)Nc3cc(Sc4ccccc4)c(O)c4ccccc34)ccc2N1] | C25H20N2O4S2 |
| 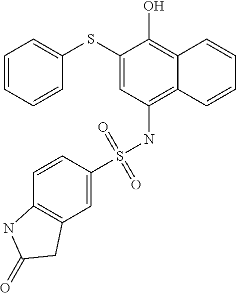 [SMILES: O=C1Cc2cc(S(=O)(=O)Nc3cc(Sc4ccccc4)c(O)c4ccccc34)ccc2N1] | C24H18N2O4S2 |
| 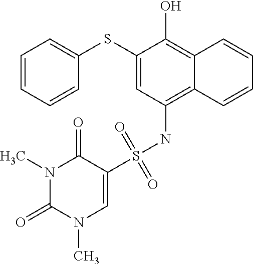 [SMILES: Cn1cc(S(=O)(=O)Nc2cc(Sc3ccccc3)c(O)c3ccccc23)c(=O)n(C)c1=O] | C22H19N3O5S2 |
| 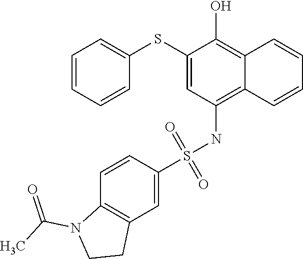 [SMILES: CC(=O)N1CCc2cc(S(=O)(=O)Nc3cc(Sc4ccccc4)c(O)c4ccccc34)ccc21] | C26H22N2O4S2 |
| 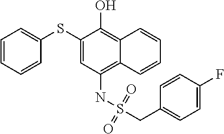 [SMILES: O=S(=O)(Cc1ccc(F)cc1)Nc1cc(Sc2ccccc2)c(O)c2ccccc12] | C23H18FNO3S2 |
| 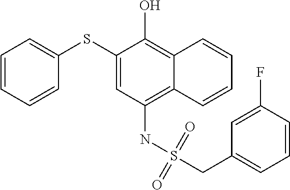 [SMILES: O=S(=O)(Cc1cccc(F)c1)Nc1cc(Sc2ccccc2)c(O)c2ccccc12] | C23H18FNO3S2 |
| 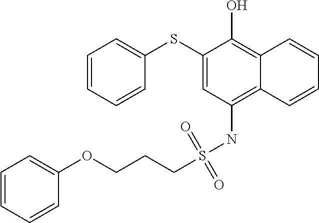 [SMILES: O=S(=O)(CCCOc1ccccc1)Nc1cc(Sc2ccccc2)c(O)c2ccccc12] | C25H23NO4S2 |
| 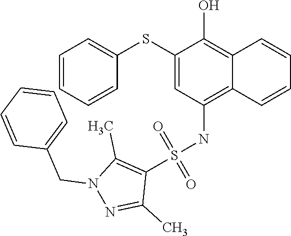 [SMILES: Cc1nn(Cc2ccccc2)c(C)c1S(=O)(=O)Nc1cc(Sc2ccccc2)c(O)c2ccccc12] | C28H25N3O3S2 |
| 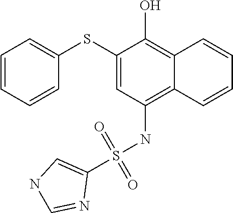 [SMILES: O=S(=O)(Nc1cc(Sc2ccccc2)c(O)c2ccccc12)c1c[nH]cn1] | C19H15N3O3S2 |
| 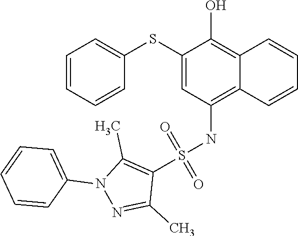 [SMILES: Cc1nn(-c2ccccc2)c(C)c1S(=O)(=O)Nc1cc(Sc2ccccc2)c(O)c2ccccc12] | C27H23N3O3S2 |
| 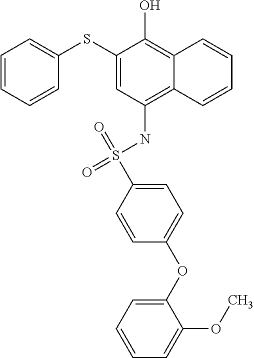 [SMILES: COc1ccccc1Oc1ccc(S(=O)(=O)Nc2cc(Sc3ccccc3)c(O)c3ccccc23)cc1] | C29H23NO5S2 |
| 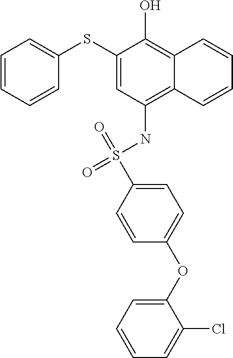 [SMILES: O=S(=O)(Nc1cc(Sc2ccccc2)c(O)c2ccccc12)c1ccc(Oc2ccccc2Cl)cc1] | C28H20ClNO4S2 |
| 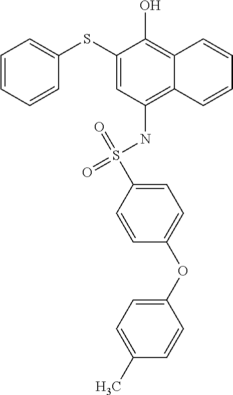 [SMILES: Cc1ccc(Oc2ccc(S(=O)(=O)Nc3cc(Sc4ccccc4)c(O)c4ccccc34)cc2)cc1] | C29H23NO4S2 |
| 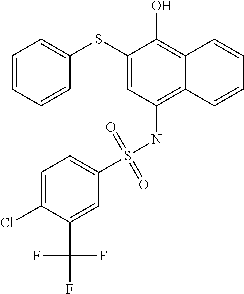 [SMILES: O=S(=O)(Nc1cc(Sc2ccccc2)c(O)c2ccccc12)c1ccc(Cl)c(C(F)(F)F)c1] | C23H15ClF3NO3S2 |
| 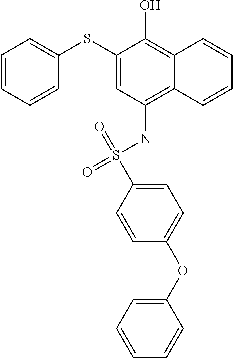 [SMILES: O=S(=O)(Nc1cc(Sc2ccccc2)c(O)c2ccccc12)c1ccc(Oc2ccccc2)cc1] | C28H21NO4S2 |
| 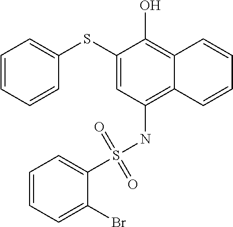 [SMILES: O=S(=O)(Nc1cc(Sc2ccccc2)c(O)c2ccccc12)c1ccccc1Br] | C22H16BrNO3S2 |
| 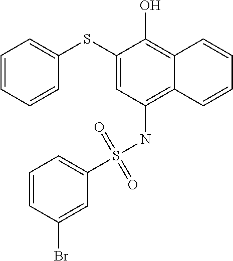 [SMILES: O=S(=O)(Nc1cc(Sc2ccccc2)c(O)c2ccccc12)c1cccc(Br)c1] | C22H16BrNO3S2 |
| 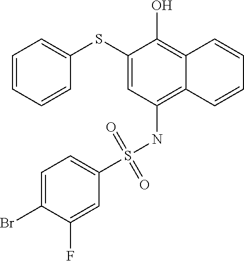 [SMILES: O=S(=O)(Nc1cc(Sc2ccccc2)c(O)c2ccccc12)c1ccc(Br)c(F)c1] | C22H15BrFNO3S2 |
| 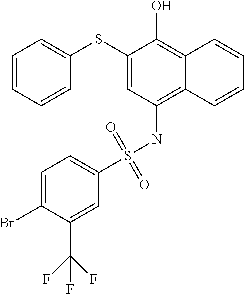 [SMILES: O=S(=O)(Nc1cc(Sc2ccccc2)c(O)c2ccccc12)c1ccc(Br)c(C(F)(F)F)c1] | C23H15BrF3NO3S2 |
| 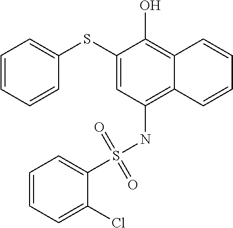 [SMILES: O=S(=O)(Nc1cc(Sc2ccccc2)c(O)c2ccccc12)c1ccccc1Cl] | C22H16ClNO3S2 |
| 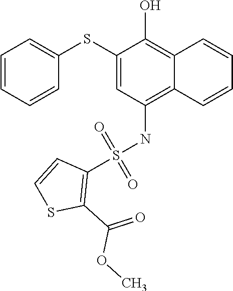 [SMILES: COC(=O)c1sccc1S(=O)(=O)Nc1cc(Sc2ccccc2)c(O)c2ccccc12] | C22H17NO5S3 |
| 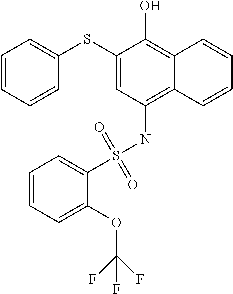 [SMILES: O=S(=O)(Nc1cc(Sc2ccccc2)c(O)c2ccccc12)c1ccccc1OC(F)(F)F] | C23H16F3NO4S2 |

TABLE 3

| Structure | Formula structure |
|---|---|
| 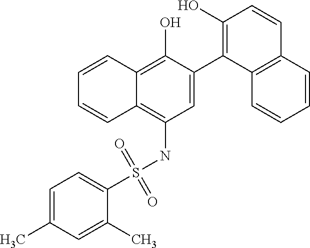 [SMILES: Cc1ccc(S(=O)(=O)Nc2cc(-c3c(O)ccc4ccccc34)c(O)c3ccccc23)c(C)c1] | C28H23NO4S |
| 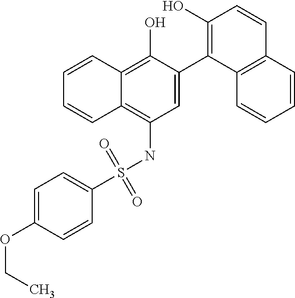 [SMILES: CCOc1ccc(S(=O)(=O)Nc2cc(-c3c(O)ccc4ccccc34)c(O)c3ccccc23)cc1] | C28H23NO5S |
| 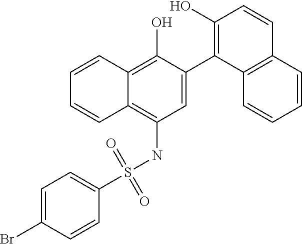 [SMILES: O=S(=O)(Nc1cc(-c2c(O)ccc3ccccc23)c(O)c2ccccc12)c1ccc(Br)cc1] | C26H18BrNO4S |
| 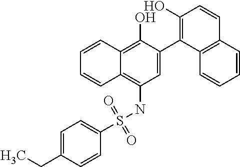 [SMILES: CCc1ccc(S(=O)(=O)Nc2cc(-c3c(O)ccc4ccccc34)c(O)c3ccccc23)cc1] | C28H23NO4S |
| 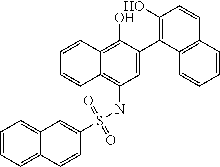 [SMILES: O=S(=O)(Nc1cc(-c2c(O)ccc3ccccc23)c(O)c2ccccc12)c1ccc2ccccc2c1] | C30H21NO4S |
| 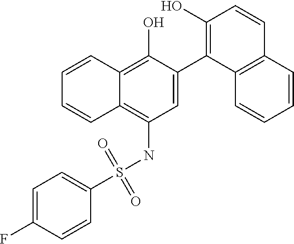 [SMILES: O=S(=O)(Nc1cc(-c2c(O)ccc3ccccc23)c(O)c2ccccc12)c1ccc(F)cc1] | C26H18FNO4S |
| 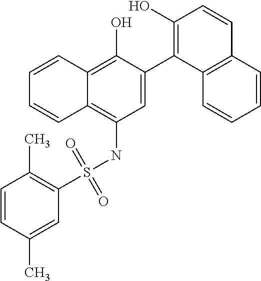 [SMILES: Cc1ccc(C)c(S(=O)(=O)Nc2cc(-c3c(O)ccc4ccccc34)c(O)c3ccccc23)c1] | C28H23NO4S |
| 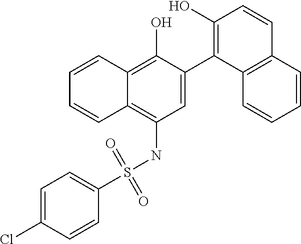 [SMILES: O=S(=O)(Nc1cc(-c2c(O)ccc3ccccc23)c(O)c2ccccc12)c1ccc(Cl)cc1] | C26H18ClNO4S |
| 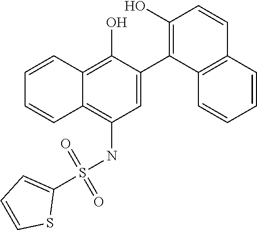 [SMILES: O=S(=O)(Nc1cc(-c2c(O)ccc3ccccc23)c(O)c2ccccc12)c1cccs1] | C24H17NO4S2 |
| 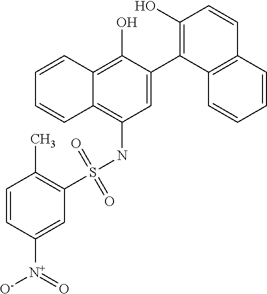 [SMILES: Cc1ccc([N+](=O)[O-])cc1S(=O)(=O)Nc1cc(-c2c(O)ccc3ccccc23)c(O)c2ccccc12] | C27H20N2O6S |
| 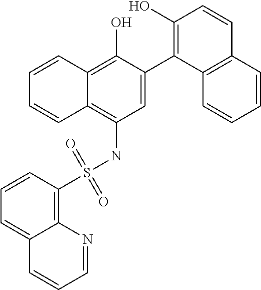 [SMILES: O=S(=O)(Nc1cc(-c2c(O)ccc3ccccc23)c(O)c2ccccc12)c1cccc2cccnc12] | C29H20N2O4S |
| 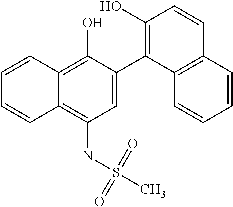 [SMILES: CS(=O)(=O)Nc1cc(-c2c(O)ccc3ccccc23)c(O)c2ccccc12] | C21H17NO4S |
| 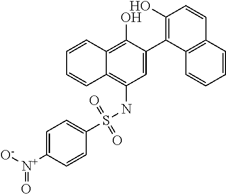 [SMILES: O=[N+]([O-])c1ccc(S(=O)(=O)Nc2cc(-c3c(O)ccc4ccccc34)c(O)c3ccccc23)cc1] | C26H18N2O6S |
| 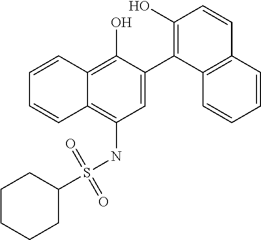 [SMILES: O=S(=O)(Nc1cc(-c2c(O)ccc3ccccc23)c(O)c2ccccc12)C1CCCCC1] | C26H25NO4S |
| 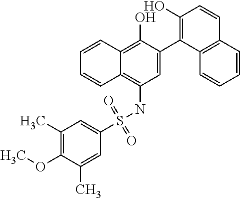 [SMILES: COc1c(C)cc(S(=O)(=O)Nc2cc(-c3c(O)ccc4ccccc34)c(O)c3ccccc23)cc1C] | C29H25NO5S |
| 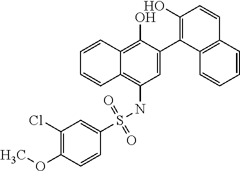 [SMILES: COc1ccc(S(=O)(=O)Nc2cc(-c3c(O)ccc4ccccc34)c(O)c3ccccc23)cc1Cl] | C27H20ClNO5S |
| 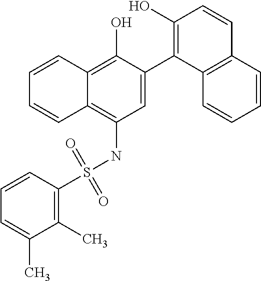 [SMILES: Cc1cccc(S(=O)(=O)Nc2cc(-c3c(O)ccc4ccccc34)c(O)c3ccccc23)c1C] | C28H23NO4S |
| 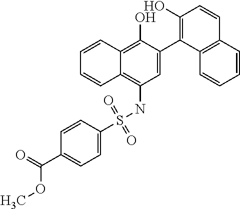 [SMILES: COC(=O)c1ccc(S(=O)(=O)Nc2cc(-c3c(O)ccc4ccccc34)c(O)c3ccccc23)cc1] | C28H21NO6S |
| 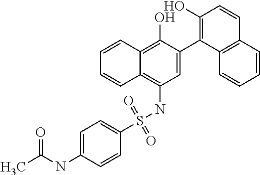 [SMILES: CC(=O)Nc1ccc(S(=O)(=O)Nc2cc(-c3c(O)ccc4ccccc34)c(O)c3ccccc23)cc1] | C28H22N2O5S |
| 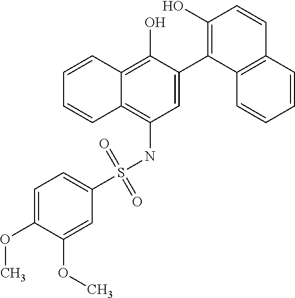 [SMILES: COc1ccc(S(=O)(=O)Nc2cc(-c3c(O)ccc4ccccc34)c(O)c3ccccc23)cc1OC] | C28H23NO6S |
| 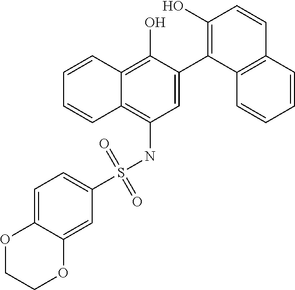 [SMILES: O=S(=O)(Nc1cc(-c2c(O)ccc3ccccc23)c(O)c2ccccc12)c1ccc2c(c1)OCCO2] | C28H21NO6S |
| 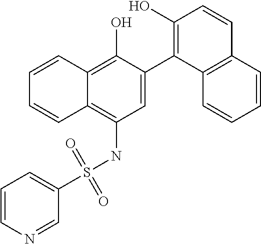 [SMILES: O=S(=O)(Nc1cc(-c2c(O)ccc3ccccc23)c(O)c2ccccc12)c1cccnc1] | C25H18N2O4S |
| 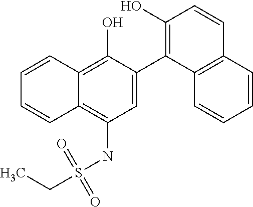 [SMILES: CCS(=O)(=O)Nc1cc(-c2c(O)ccc3ccccc23)c(O)c2ccccc12] | C22H19NO4S |
| 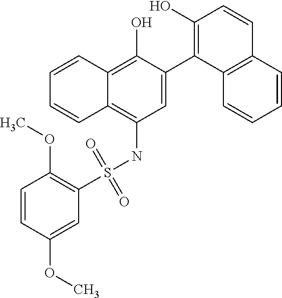 [SMILES: COc1ccc(OC)c(S(=O)(=O)Nc2cc(-c3c(O)ccc4ccccc34)c(O)c3ccccc23)c1] | C28H23NO6S |
| 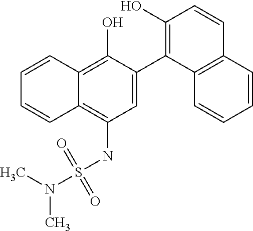 [SMILES: CN(C)S(=O)(=O)Nc1cc(-c2c(O)ccc3ccccc23)c(O)c2ccccc12] | C22H20N2O4S |
| 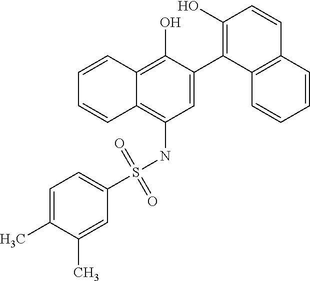 [SMILES: Cc1ccc(S(=O)(=O)Nc2cc(-c3c(O)ccc4ccccc34)c(O)c3ccccc23)cc1C] | C28H23NO4S |
| 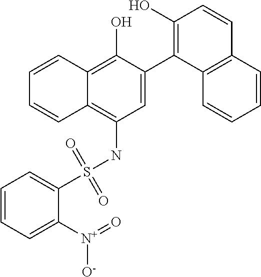 [SMILES: O=[N+]([O-])c1ccccc1S(=O)(=O)Nc1cc(-c2c(O)ccc3ccccc23)c(O)c2ccccc12] | C26H18N2O6S |
| 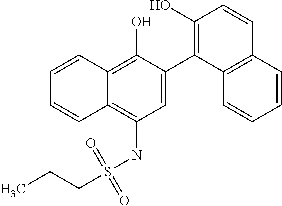 [SMILES: CCCS(=O)(=O)Nc1cc(-c2c(O)ccc3ccccc23)c(O)c2ccccc12] | C23H21NO4S |
| 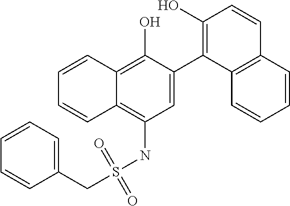 [SMILES: O=S(=O)(Cc1ccccc1)Nc1cc(-c2c(O)ccc3ccccc23)c(O)c2ccccc12] | C27H21NO4S |
| 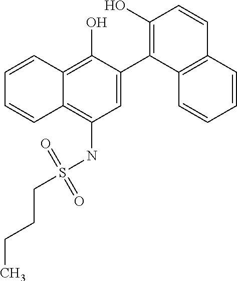 [SMILES: CCCCS(=O)(=O)Nc1cc(-c2c(O)ccc3ccccc23)c(O)c2ccccc12] | C24H23NO4S |
| 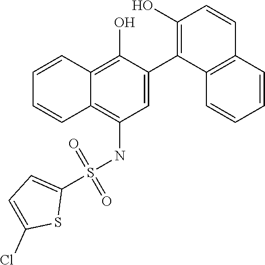 [SMILES: O=S(=O)(Nc1cc(-c2c(O)ccc3ccccc23)c(O)c2ccccc12)c1ccc(Cl)s1] | C24H16ClNO4S2 |
| 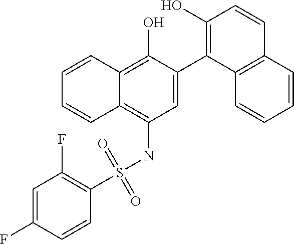 [SMILES: O=S(=O)(Nc1cc(-c2c(O)ccc3ccccc23)c(O)c2ccccc12)c1ccc(F)cc1F] | C26H17F2NO4S |
| 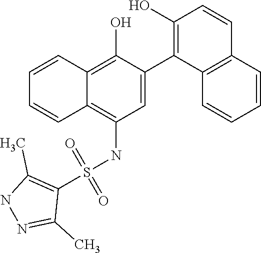 [SMILES: Cc1n[nH]c(C)c1S(=O)(=O)Nc1cc(-c2c(O)ccc3ccccc23)c(O)c2ccccc12] | C25H21N3O4S |
| 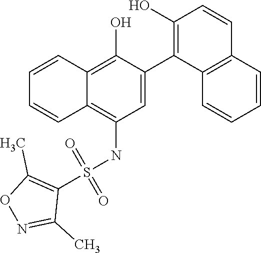 [SMILES: Cc1noc(C)c1S(=O)(=O)Nc1cc(-c2c(O)ccc3ccccc23)c(O)c2ccccc12] | C25H20N2O5S |
| 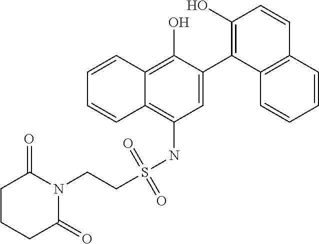 [SMILES: O=C1CCCC(=O)N1CCS(=O)(=O)Nc1cc(-c2c(O)ccc3ccccc23)c(O)c2ccccc12] | C27H24N2O6S |
| 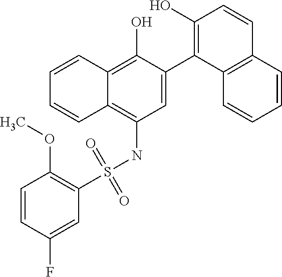 [SMILES: COc1ccc(F)cc1S(=O)(=O)Nc1cc(-c2c(O)ccc3ccccc23)c(O)c2ccccc12] | C27H20FNO5S |
| 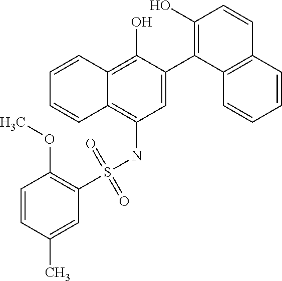 [SMILES: COc1ccc(C)cc1S(=O)(=O)Nc1cc(-c2c(O)ccc3ccccc23)c(O)c2ccccc12] | C28H23NO5S |
| 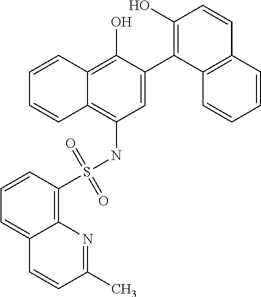 [SMILES: Cc1ccc2cccc(S(=O)(=O)Nc3cc(-c4c(O)ccc5ccccc45)c(O)c4ccccc34)c2n1] | C30H22N2O4S |
| 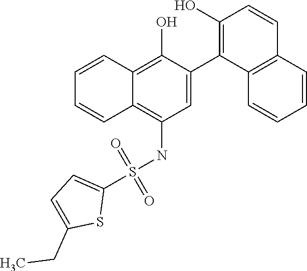 [SMILES: CCc1ccc(S(=O)(=O)Nc2cc(-c3c(O)ccc4ccccc34)c(O)c3ccccc23)s1] | C26H21NO4S2 |
| 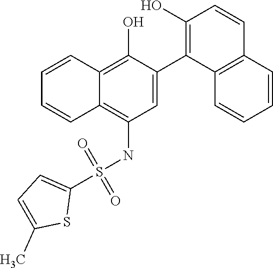 [SMILES: Cc1ccc(S(=O)(=O)Nc2cc(-c3c(O)ccc4ccccc34)c(O)c3ccccc23)s1] | C25H19NO4S2 |
| 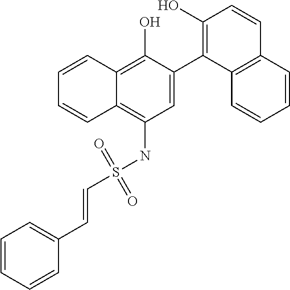 [SMILES: O=S(=O)(/C=C/c1ccccc1)Nc1cc(-c2c(O)ccc3ccccc23)c(O)c2ccccc12] | C28H21NO4S |
| 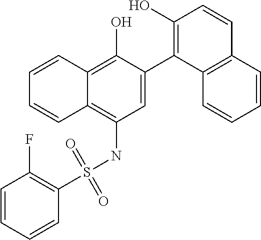 [SMILES: O=S(=O)(Nc1cc(-c2c(O)ccc3ccccc23)c(O)c2ccccc12)c1ccccc1F] | C26H18FNO4S |
| 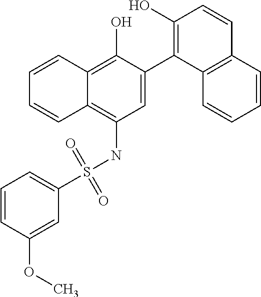 [SMILES: COc1cccc(S(=O)(=O)Nc2cc(-c3c(O)ccc4ccccc34)c(O)c3ccccc23)c1] | C27H21NO5S |
| 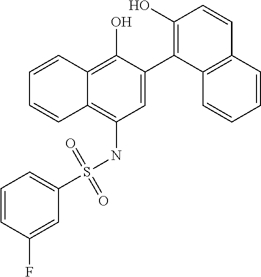 [SMILES: O=S(=O)(Nc1cc(-c2c(O)ccc3ccccc23)c(O)c2ccccc12)c1cccc(F)c1] | C26H18FNO4S |
| 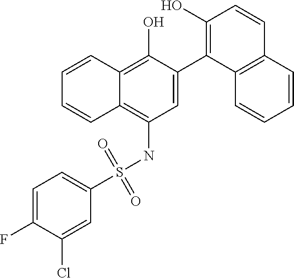 [SMILES: O=S(=O)(Nc1cc(-c2c(O)ccc3ccccc23)c(O)c2ccccc12)c1ccc(F)c(Cl)c1] | C26H17ClFNO4S |
| 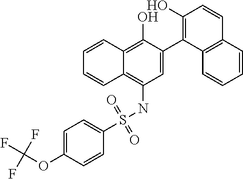 [SMILES: O=S(=O)(Nc1cc(-c2c(O)ccc3ccccc23)c(O)c2ccccc12)c1ccc(OC(F)(F)F)cc1] | C27H18F3NO5S |
| 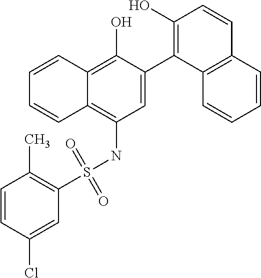 [SMILES: Cc1ccc(Cl)cc1S(=O)(=O)Nc1cc(-c2c(O)ccc3ccccc23)c(O)c2ccccc12] | C27H20ClNO4S |
| 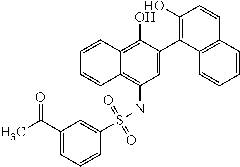 [SMILES: CC(=O)c1cccc(S(=O)(=O)Nc2cc(-c3c(O)ccc4ccccc34)c(O)c3ccccc23)c1] | C28H21NO5S |
| 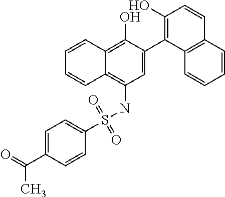 [SMILES: CC(=O)c1ccc(S(=O)(=O)Nc2cc(-c3c(O)ccc4ccccc34)c(O)c3ccccc23)cc1] | C28H21NO5S |
| 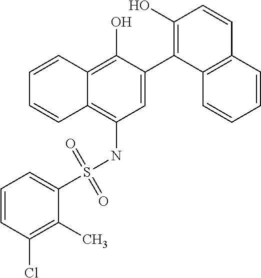 [SMILES: Cc1c(Cl)cccc1S(=O)(=O)Nc1cc(-c2c(O)ccc3ccccc23)c(O)c2ccccc12] | C27H20ClNO4S |
| 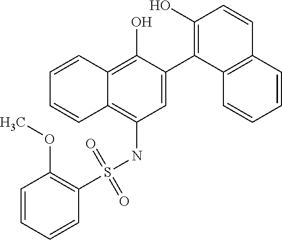 [SMILES: COc1ccccc1S(=O)(=O)Nc1cc(-c2c(O)ccc3ccccc23)c(O)c2ccccc12] | C27H21NO5S |
| 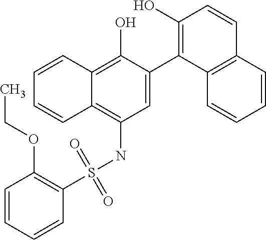 [SMILES: CCOc1ccccc1S(=O)(=O)Nc1cc(-c2c(O)ccc3ccccc23)c(O)c2ccccc12] | C28H23NO5S |
| 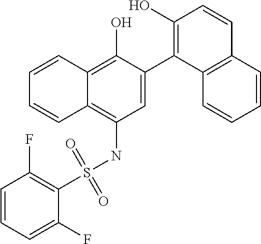 [SMILES: O=S(=O)(Nc1cc(-c2c(O)ccc3ccccc23)c(O)c2ccccc12)c1c(F)cccc1F] | C26H17F2NO4S |
| 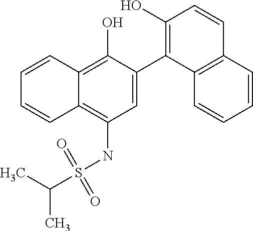 [SMILES: CC(C)S(=O)(=O)Nc1cc(-c2c(O)ccc3ccccc23)c(O)c2ccccc12] | C23H21NO4S |
| 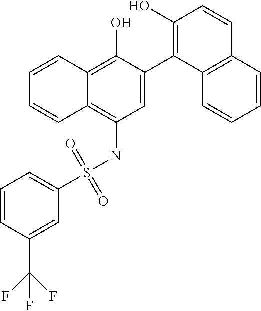 [SMILES: O=S(=O)(Nc1cc(-c2c(O)ccc3ccccc23)c(O)c2ccccc12)c1cccc(C(F)(F)F)c1] | C27H18F3NO4S |
| 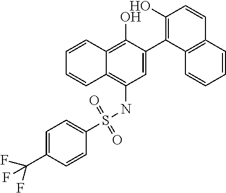 [SMILES: O=S(=O)(Nc1cc(-c2c(O)ccc3ccccc23)c(O)c2ccccc12)c1ccc(C(F)(F)F)cc1] | C27H18F3NO4S |
| 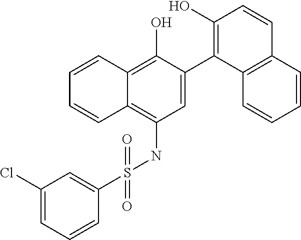 [SMILES: O=S(=O)(Nc1cc(-c2c(O)ccc3ccccc23)c(O)c2ccccc12)c1cccc(Cl)c1] | C26H18ClNO4S |
| 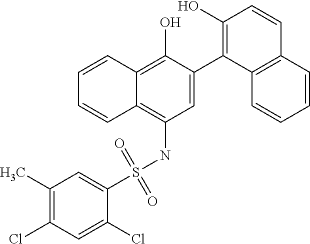 [SMILES: Cc1cc(S(=O)(=O)Nc2cc(-c3c(O)ccc4ccccc34)c(O)c3ccccc23)c(Cl)cc1Cl] | C27H19 Cl2NO4S |
| 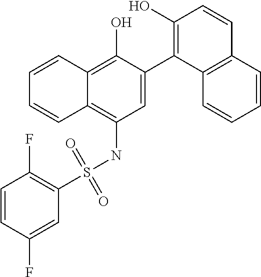 [SMILES: O=S(=O)(Nc1cc(-c2c(O)ccc3ccccc23)c(O)c2ccccc12)c1cc(F)ccc1F] | C26H17F2NO4S |
| 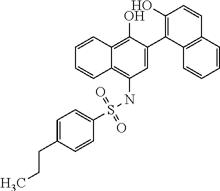 [SMILES: CCCc1ccc(S(=O)(=O)Nc2cc(-c3c(O)ccc4ccccc34)c(O)c3ccccc23)cc1] | C29H25NO4S |
| 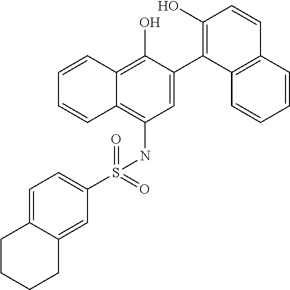 [SMILES: O=S(=O)(Nc1cc(-c2c(O)ccc3ccccc23)c(O)c2ccccc12)c1ccc2c(c1)CCCC2] | C30H25NO4S |
| 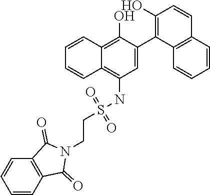 [SMILES: O=C1c2ccccc2C(=O)N1CCS(=O)(=O)Nc1cc(-c2c(O)ccc3ccccc23)c(O)c2ccccc12] | C30H22N2O6S |
| 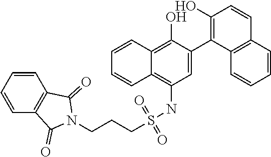 [SMILES: O=C1c2ccccc2C(=O)N1CCCS(=O)(=O)Nc1cc(-c2c(O)ccc3ccccc23)c(O)c2ccccc12] | C31H24N2O6S |
| 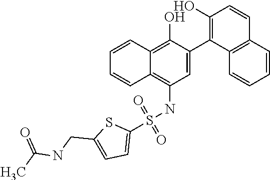 [SMILES: CC(=O)NCc1ccc(S(=O)(=O)Nc2cc(-c3c(O)ccc4ccccc34)c(O)c3ccccc23)s1] | C27H22N2O5S2 |
| 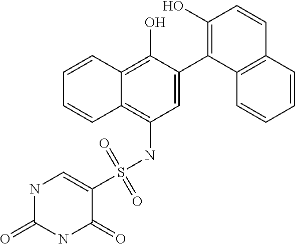 [SMILES: O=c1[nH]cc(S(=O)(=O)Nc2cc(-c3c(O)ccc4ccccc34)c(O)c3ccccc23)c(=O)[nH]1] | C24H17N3O6S |
| 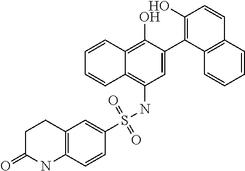 [SMILES: O=C1CCc2cc(S(=O)(=O)Nc3cc(-c4c(O)ccc5ccccc45)c(O)c4ccccc34)ccc2N1] | C29H22N2O5S |
| 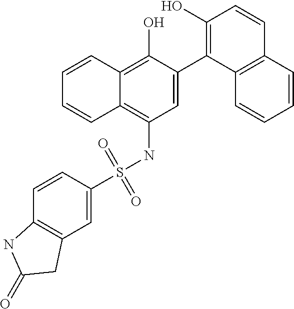 [SMILES: O=C1Cc2cc(S(=O)(=O)Nc3cc(-c4c(O)ccc5ccccc45)c(O)c4ccccc34)ccc2N1] | C28H20N2O5S |
| 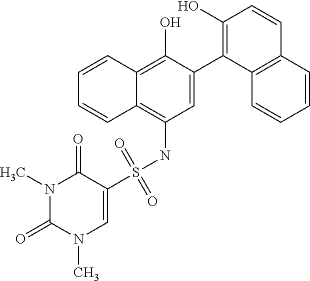 [SMILES: Cn1cc(S(=O)(=O)Nc2cc(-c3c(O)ccc4ccccc34)c(O)c3ccccc23)c(=O)n(C)c1=O] | C26H21N3O6S |
| 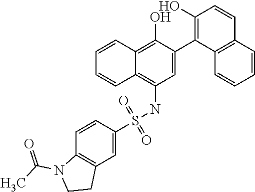 [SMILES: CC(=O)N1CCc2cc(S(=O)(=O)Nc3cc(-c4c(O)ccc5ccccc45)c(O)c4ccccc34)ccc21] | C30H24N2O5S |
| 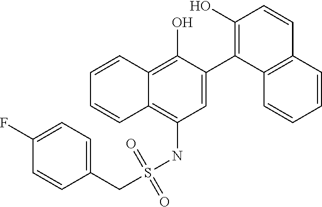 [SMILES: O=S(=O)(Cc1ccc(F)cc1)Nc1cc(-c2c(O)ccc3ccccc23)c(O)c2ccccc12] | C27H20FNO4S |
| 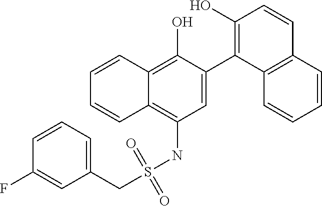 [SMILES: O=S(=O)(Cc1cccc(F)c1)Nc1cc(-c2c(O)ccc3ccccc23)c(O)c2ccccc12] | C27H20FNO4S |
| 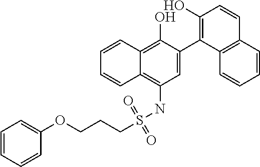 [SMILES: O=S(=O)(CCCOc1ccccc1)Nc1cc(-c2c(O)ccc3ccccc23)c(O)c2ccccc12] | C29H25NO5S |
| 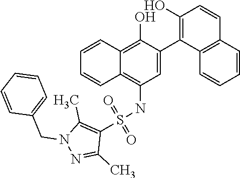 [SMILES: Cc1nn(Cc2ccccc2)c(C)c1S(=O)(=O)Nc1cc(-c2c(O)ccc3ccccc23)c(O)c2ccccc12] | C32H27N3O4S |
| 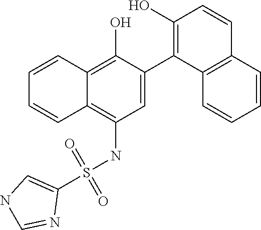 [SMILES: O=S(=O)(Nc1cc(-c2c(O)ccc3ccccc23)c(O)c2ccccc12)c1c[nH]cn1] | C23H17N3O4S |
| 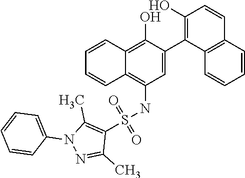 [SMILES: Cc1nn(-c2ccccc2)c(C)c1S(=O)(=O)Nc1cc(-c2c(O)ccc3ccccc23)c(O)c2ccccc12] | C31H25N3O4S |
| 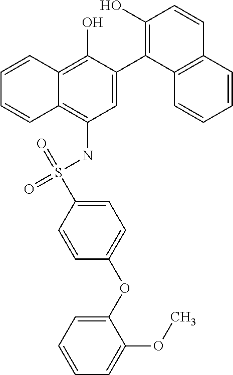 [SMILES: COc1ccccc1Oc1ccc(S(=O)(=O)Nc2cc(-c3c(O)ccc4ccccc34)c(O)c3ccccc23)cc1] | C33H25NO6S |
| 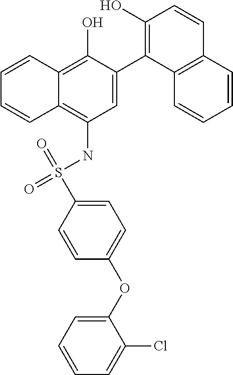 [SMILES: O=S(=O)(Nc1cc(-c2c(O)ccc3ccccc23)c(O)c2ccccc12)c1ccc(Oc2ccccc2Cl)cc1] | C32H22ClNO5S |
| 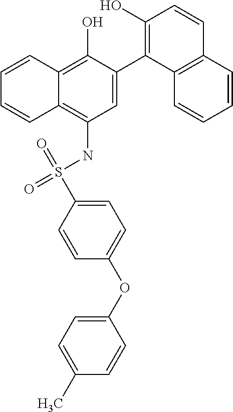 [SMILES: Cc1ccc(Oc2ccc(S(=O)(=O)Nc3cc(-c4c(O)ccc5ccccc45)c(O)c4ccccc34)cc2)cc1] | C33H25NO5S |
| 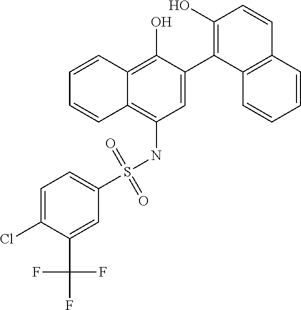 [SMILES: O=S(=O)(Nc1cc(-c2c(O)ccc3ccccc23)c(O)c2ccccc12)c1ccc(Cl)c(C(F)(F)F)c1] | C27H17Cl F3NO4S |
| 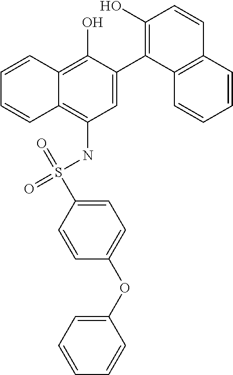 [SMILES: O=S(=O)(Nc1cc(-c2c(O)ccc3ccccc23)c(O)c2ccccc12)c1ccc(Oc2ccccc2)cc1] | C32H23NO5S |
| 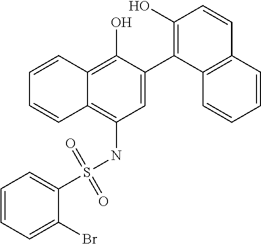 [SMILES: O=S(=O)(Nc1cc(-c2c(O)ccc3ccccc23)c(O)c2ccccc12)c1ccccc1Br] | C26H18BrNO4S |
| 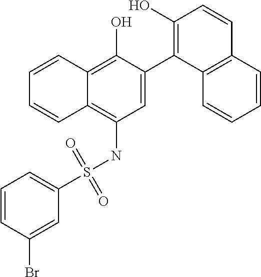 [SMILES: O=S(=O)(Nc1cc(-c2c(O)ccc3ccccc23)c(O)c2ccccc12)c1cccc(Br)c1] | C26H18BrNO4S |
| 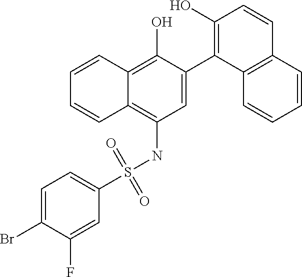 [SMILES: O=S(=O)(Nc1cc(-c2c(O)ccc3ccccc23)c(O)c2ccccc12)c1ccc(Br)c(F)c1] | C26H17Br FNO4S |
| 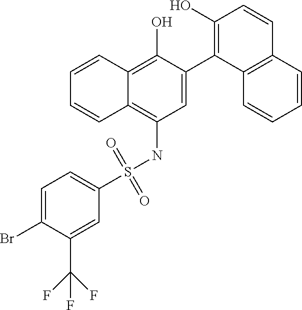 [SMILES: O=S(=O)(Nc1cc(-c2c(O)ccc3ccccc23)c(O)c2ccccc12)c1ccc(Br)c(C(F)(F)F)c1] | C27H17Br F3NO4S |
| 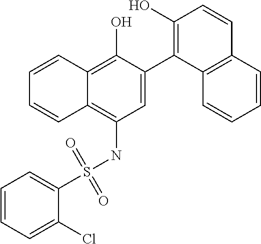 [SMILES: O=S(=O)(Nc1cc(-c2c(O)ccc3ccccc23)c(O)c2ccccc12)c1ccccc1Cl] | C26H18ClNO4S |
| 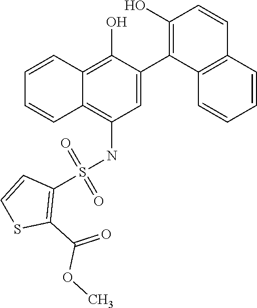 [SMILES: COC(=O)c1sccc1S(=O)(=O)Nc1cc(-c2c(O)ccc3ccccc23)c(O)c2ccccc12] | C26H19NO6S2 |
| 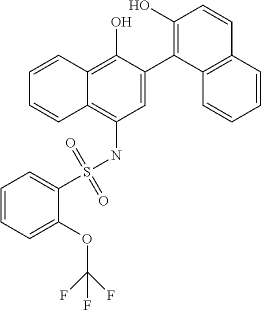 [SMILES: O=S(=O)(Nc1cc(-c2c(O)ccc3ccccc23)c(O)c2ccccc12)c1ccccc1OC(F)(F)F] | C27H18F3NO5S |

TABLE 4

| Structure | Formula structure |
|---|---|
| 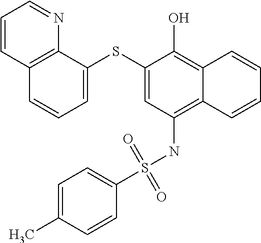 [SMILES: Cc1ccc(S(=O)(=O)Nc2cc(Sc3cccc4cccnc34)c(O)c3ccccc23)cc1] | C26H20N2O3S2 |
| 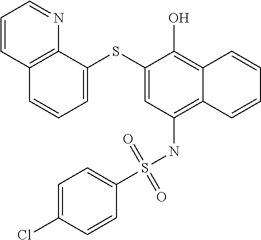 [SMILES: O=S(=O)(Nc1cc(Sc2cccc3cccnc23)c(O)c2ccccc12)c1ccc(Cl)cc1] | C25H17ClN2O3S2 |
| 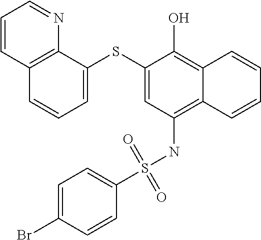 [SMILES: O=S(=O)(Nc1cc(Sc2cccc3cccnc23)c(O)c2ccccc12)c1ccc(Br)cc1] | C25H17BrN2O3S2 |
| 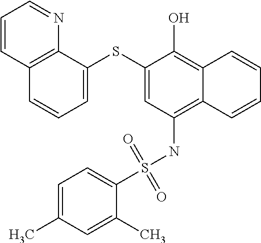 [SMILES: Cc1ccc(S(=O)(=O)Nc2cc(Sc3cccc4cccnc34)c(O)c3ccccc23)c(C)c1] | C27H22N2O3S2 |
| 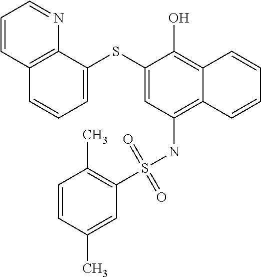 [SMILES: Cc1ccc(C)c(S(=O)(=O)Nc2cc(Sc3cccc4cccnc34)c(O)c3ccccc23)c1] | C27H22N2O3S2 |
| 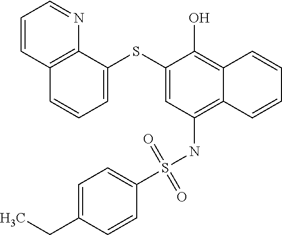 [SMILES: CCc1ccc(S(=O)(=O)Nc2cc(Sc3cccc4cccnc34)c(O)c3ccccc23)cc1] | C27H22N2O3S2 |
| 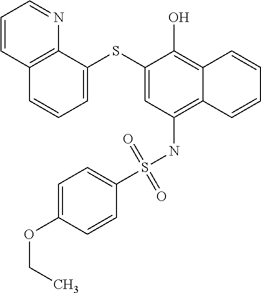 [SMILES: CCOc1ccc(S(=O)(=O)Nc2cc(Sc3cccc4cccnc34)c(O)c3ccccc23)cc1] | C27H22N2O4S2 |
| 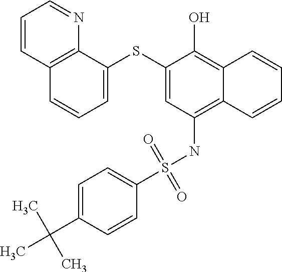 [SMILES: CC(C)(C)c1ccc(S(=O)(=O)Nc2cc(Sc3cccc4cccnc34)c(O)c3ccccc23)cc1] | C29H26N2O3S2 |
| 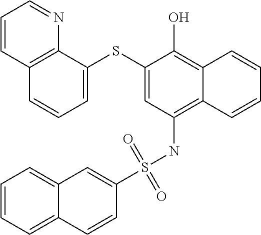 [SMILES: O=S(=O)(Nc1cc(Sc2cccc3cccnc23)c(O)c2ccccc12)c1ccc2ccccc2c1] | C29H20N2O3S2 |
| 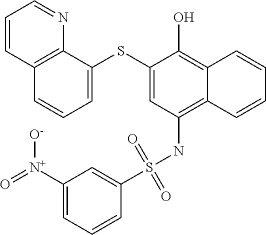 [SMILES: O=[N+]([O-])c1cccc(S(=O)(=O)Nc2cc(Sc3cccc4cccnc34)c(O)c3ccccc23)c1] | C25H17N3O5S2 |
| 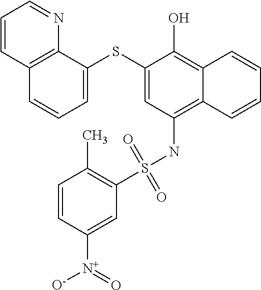 [SMILES: Cc1ccc([N+](=O)[O-])cc1S(=O)(=O)Nc1cc(Sc2cccc3cccnc23)c(O)c2ccccc12] | C26H19N3O5S2 |
| 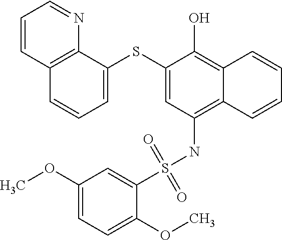 [SMILES: COc1ccc(OC)c(S(=O)(=O)Nc2cc(Sc3cccc4cccnc34)c(O)c3ccccc23)c1] | C27H22N2O5S2 |
| 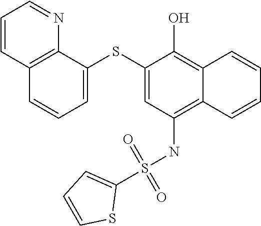 [SMILES: O=S(=O)(Nc1cc(Sc2cccc3cccnc23)c(O)c2ccccc12)c1cccs1] | C23H16N2O3S3 |
| 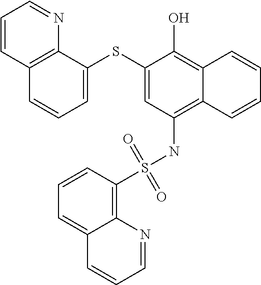 [SMILES: O=S(=O)(Nc1cc(Sc2cccc3cccnc23)c(O)c2ccccc12)c1cccc2cccnc12] | C28H19N3O3S2 |
| 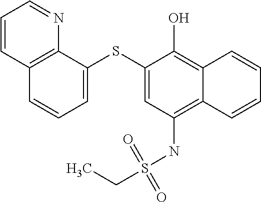 [SMILES: CCS(=O)(=O)Nc1cc(Sc2cccc3cccnc23)c(O)c2ccccc12] | C21H18N2O3S2 |
| 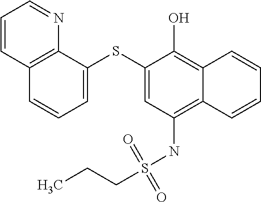 [SMILES: CCCS(=O)(=O)Nc1cc(Sc2cccc3cccnc23)c(O)c2ccccc12] | C22H20N2O3S2 |
| 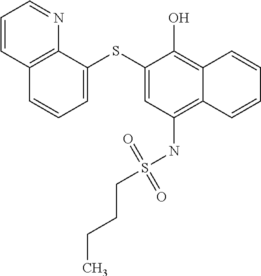 [SMILES: CCCCS(=O)(=O)Nc1cc(Sc2cccc3cccnc23)c(O)c2ccccc12] | C23H22N2O3S2 |
| 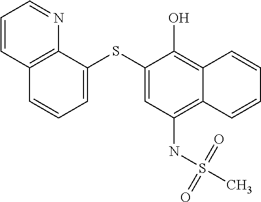 [SMILES: CS(=O)(=O)Nc1cc(Sc2cccc3cccnc23)c(O)c2ccccc12] | C20H16N2O3S2 |
| 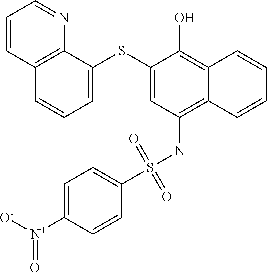 [SMILES: O=[N+]([O-])c1ccc(S(=O)(=O)Nc2cc(Sc3cccc4cccnc34)c(O)c3ccccc23)cc1] | C25H17N3O5S2 |
| 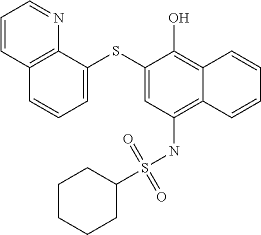 [SMILES: O=S(=O)(Nc1cc(Sc2cccc3cccnc23)c(O)c2ccccc12)C1CCCCC1] | C25H24N2O3S2 |
| 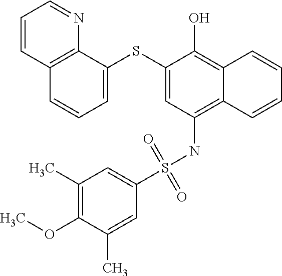 [SMILES: COc1c(C)cc(S(=O)(=O)Nc2cc(Sc3cccc4cccnc34)c(O)c3ccccc23)cc1C] | C28H24N2O4S2 |
| 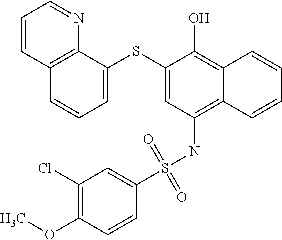 [SMILES: COc1ccc(S(=O)(=O)Nc2cc(Sc3cccc4cccnc34)c(O)c3ccccc23)cc1Cl] | C26H19ClN2O4S2 |
| 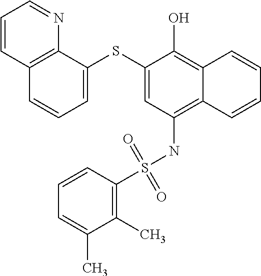 [SMILES: Cc1cccc(S(=O)(=O)Nc2cc(Sc3cccc4cccnc34)c(O)c3ccccc23)c1C] | C27H22N2O3S2 |
| 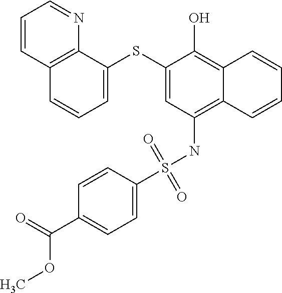 [SMILES: COC(=O)c1ccc(S(=O)(=O)Nc2cc(Sc3cccc4cccnc34)c(O)c3ccccc23)cc1] | C27H20N2O5S2 |
| 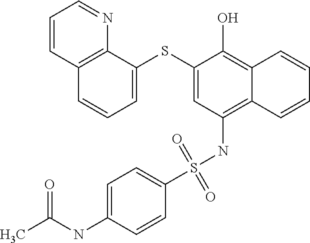 [SMILES: CC(=O)Nc1ccc(S(=O)(=O)Nc2cc(Sc3cccc4cccnc34)c(O)c3ccccc23)cc1] | C27H21N3O4S2 |
| 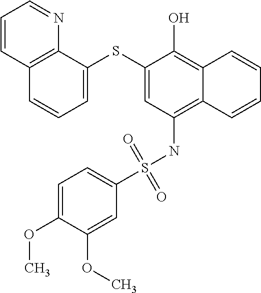 [SMILES: COc1ccc(S(=O)(=O)Nc2cc(Sc3cccc4cccnc34)c(O)c3ccccc23)cc1OC] | C27H22N2O5S2 |
| 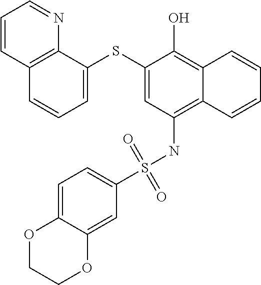 [SMILES: O=S(=O)(Nc1cc(Sc2cccc3cccnc23)c(O)c2ccccc12)c1ccc2c(c1)OCCO2] | C27H20N2O5S2 |
| 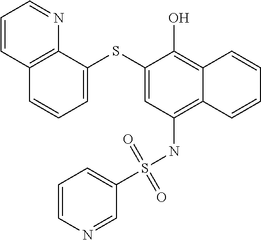 [SMILES: O=S(=O)(Nc1cc(Sc2cccc3cccnc23)c(O)c2ccccc12)c1cccnc1] | C24H17N3O3S2 |
| 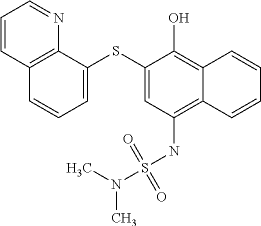 [SMILES: CN(C)S(=O)(=O)Nc1cc(Sc2cccc3cccnc23)c(O)c2ccccc12] | C21H19N3O3S2 |
| 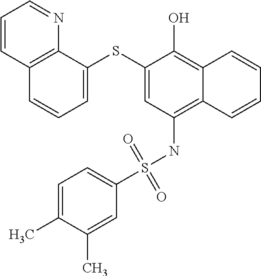 [SMILES: Cc1ccc(S(=O)(=O)Nc2cc(Sc3cccc4cccnc34)c(O)c3ccccc23)cc1C] | C27H22N2O3S2 |
| 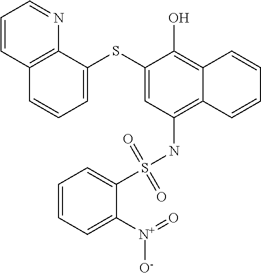 [SMILES: O=[N+]([O-])c1ccccc1S(=O)(=O)Nc1cc(Sc2cccc3cccnc23)c(O)c2ccccc12] | C25H17N3O5S2 |
| 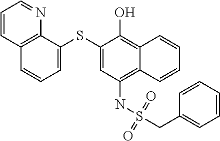 [SMILES: O=S(=O)(Cc1ccccc1)Nc1cc(Sc2cccc3cccnc23)c(O)c2ccccc12] | C26H20N2O3S2 |
| 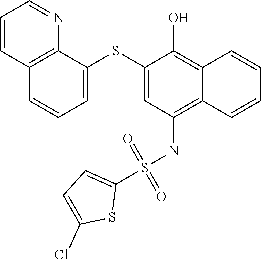 [SMILES: O=S(=O)(Nc1cc(Sc2cccc3cccnc23)c(O)c2ccccc12)c1ccc(Cl)s1] | C23H15ClN2O3S3 |
| 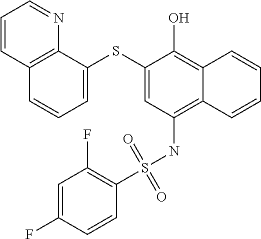 [SMILES: O=S(=O)(Nc1cc(Sc2cccc3cccnc23)c(O)c2ccccc12)c1ccc(F)cc1F] | C25H16F2N2O3S2 |
| 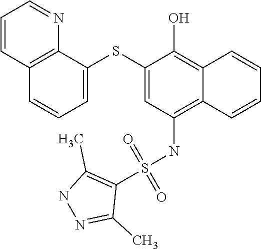 [SMILES: Cc1n[nH]c(C)c1S(=O)(=O)Nc1cc(Sc2cccc3cccnc23)c(O)c2ccccc12] | C24H20N4O3S2 |
| 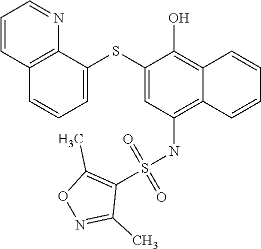 [SMILES: Cc1noc(C)c1S(=O)(=O)Nc1cc(Sc2cccc3cccnc23)c(O)c2ccccc12] | C24H19N3O4S2 |
| 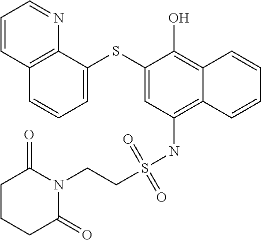 [SMILES: O=C1CCCC(=O)N1CCS(=O)(=O)Nc1cc(Sc2cccc3cccnc23)c(O)c2ccccc12] | C26H23N3O5S2 |
| 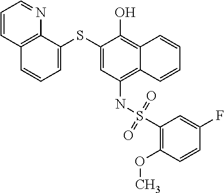 [SMILES: COc1ccc(F)cc1S(=O)(=O)Nc1cc(Sc2cccc3cccnc23)c(O)c2ccccc12] | C26H19FN2O4S2 |
| 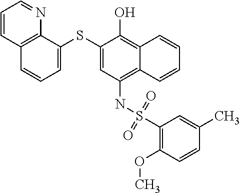 [SMILES: COc1ccc(C)cc1S(=O)(=O)Nc1cc(Sc2cccc3cccnc23)c(O)c2ccccc12] | C27H22N2O4S2 |
| 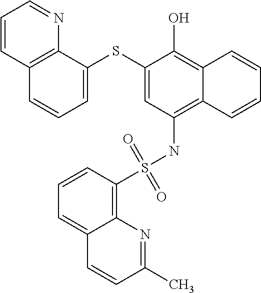 [SMILES: Cc1ccc2cccc(S(=O)(=O)Nc3cc(Sc4cccc5cccnc45)c(O)c4ccccc34)c2n1] | C29H21N3O3S2 |
| 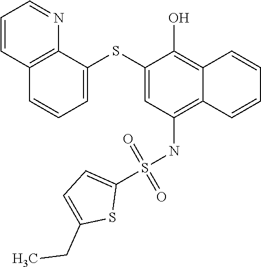 [SMILES: CCc1ccc(S(=O)(=O)Nc2cc(Sc3cccc4cccnc34)c(O)c3ccccc23)s1] | C25H20N2O3S3 |
| 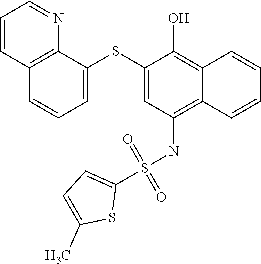 [SMILES: Cc1ccc(S(=O)(=O)Nc2cc(Sc3cccc4cccnc34)c(O)c3ccccc23)s1] | C24H18N2O3S3 |
| 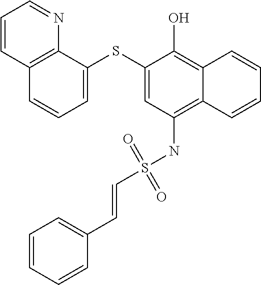 [SMILES: O=S(=O)(/C=C/c1ccccc1)Nc1cc(Sc2cccc3cccnc23)c(O)c2ccccc12] | C27H20N2O3S2 |
| 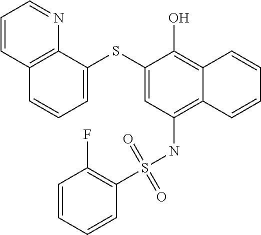 [SMILES: O=S(=O)(Nc1cc(Sc2cccc3cccnc23)c(O)c2ccccc12)c1ccccc1F] | C25H17FN2O3S2 |
| 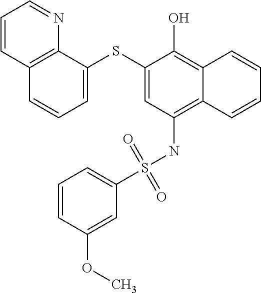 [SMILES: COc1cccc(S(=O)(=O)Nc2cc(Sc3cccc4cccnc34)c(O)c3ccccc23)c1] | C26H20N2O4S2 |
| 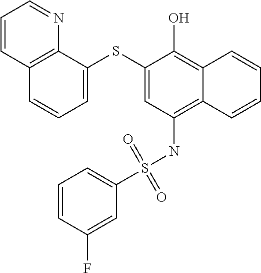 [SMILES: O=S(=O)(Nc1cc(Sc2cccc3cccnc23)c(O)c2ccccc12)c1cccc(F)c1] | C25H17FN2O3S2 |
| 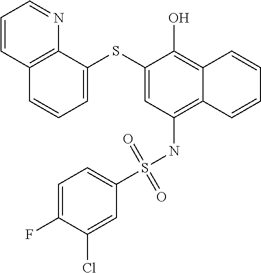 [SMILES: O=S(=O)(Nc1cc(Sc2cccc3cccnc23)c(O)c2ccccc12)c1ccc(F)c(Cl)c1] | C25H16ClFN2O3S2 |
| 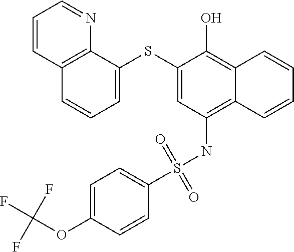 [SMILES: O=S(=O)(Nc1cc(Sc2cccc3cccnc23)c(O)c2ccccc12)c1ccc(OC(F)(F)F)cc1] | C26H17F3N2O4S2 |
| 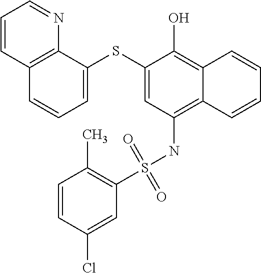 [SMILES: Cc1ccc(Cl)cc1S(=O)(=O)Nc1cc(Sc2cccc3cccnc23)c(O)c2ccccc12] | C26H19ClN2O3S2 |
| 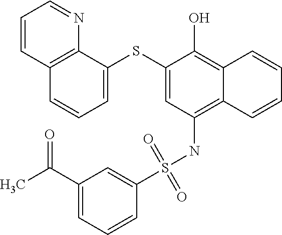 [SMILES: CC(=O)c1cccc(S(=O)(=O)Nc2cc(Sc3cccc4cccnc34)c(O)c3ccccc23)c1] | C27H20N2O4S2 |
| 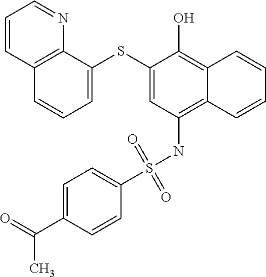 [SMILES: CC(=O)c1ccc(S(=O)(=O)Nc2cc(Sc3cccc4cccnc34)c(O)c3ccccc23)cc1] | C27H20N2O4S2 |
| 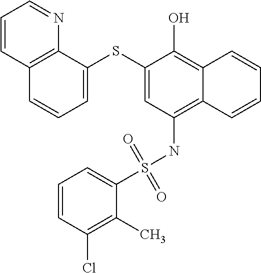 [SMILES: Cc1c(Cl)cccc1S(=O)(=O)Nc1cc(Sc2cccc3cccnc23)c(O)c2ccccc12] | C26H19ClN2O3S2 |
| 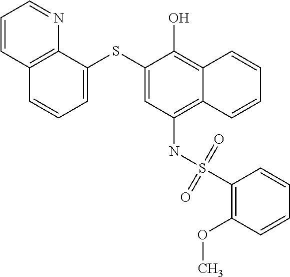 [SMILES: COc1ccccc1S(=O)(=O)Nc1cc(Sc2cccc3cccnc23)c(O)c2ccccc12] | C26H20N2O4S2 |
| 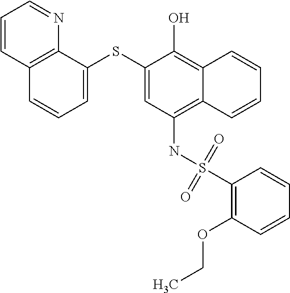 [SMILES: CCOc1ccccc1S(=O)(=O)Nc1cc(Sc2cccc3cccnc23)c(O)c2ccccc12] | C27H22N2O4S2 |
| 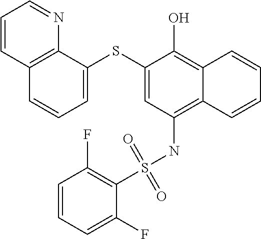 [SMILES: O=S(=O)(Nc1cc(Sc2cccc3cccnc23)c(O)c2ccccc12)c1c(F)cccc1F] | C25H16F2N2O3S2 |
| 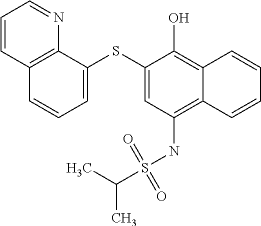 [SMILES: CC(C)S(=O)(=O)Nc1cc(Sc2cccc3cccnc23)c(O)c2ccccc12] | C22H20N2O3S2 |
| 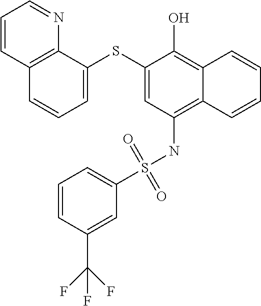 [SMILES: O=S(=O)(Nc1cc(Sc2cccc3cccnc23)c(O)c2ccccc12)c1cccc(C(F)(F)F)c1] | C26H17F3N2O3S2 |
| 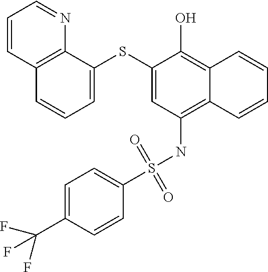 [SMILES: O=S(=O)(Nc1cc(Sc2cccc3cccnc23)c(O)c2ccccc12)c1ccc(C(F)(F)F)cc1] | C26H17F3N2O3S2 |
| 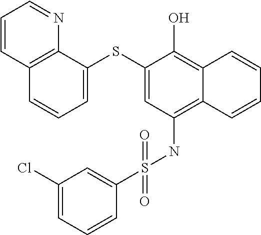 [SMILES: O=S(=O)(Nc1cc(Sc2cccc3cccnc23)c(O)c2ccccc12)c1cccc(Cl)c1] | C25H17ClN2O3S2 |
| 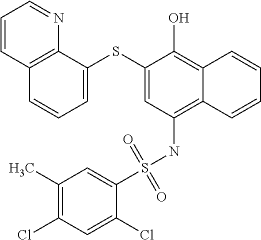 [SMILES: Cc1cc(S(=O)(=O)Nc2cc(Sc3cccc4cccnc34)c(O)c3ccccc23)c(Cl)cc1Cl] | C26H18Cl2 N2O3S2 |
| 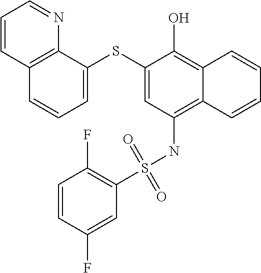 [SMILES: O=S(=O)(Nc1cc(Sc2cccc3cccnc23)c(O)c2ccccc12)c1cc(F)ccc1F] | C25H16F2N2O3S2 |
| 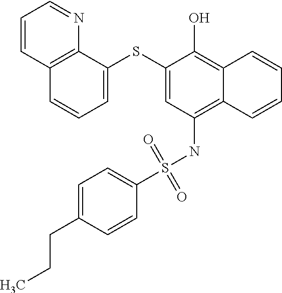 [SMILES: CCCc1ccc(S(=O)(=O)Nc2cc(Sc3cccc4cccnc34)c(O)c3ccccc23)cc1] | C28H24N2O3S2 |
| 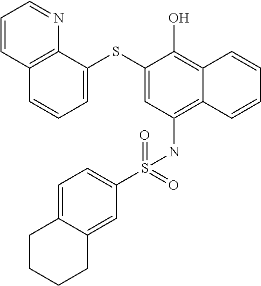 [SMILES: O=S(=O)(Nc1cc(Sc2cccc3cccnc23)c(O)c2ccccc12)c1ccc2c(c1)CCCC2] | C29H24N2O3S2 |
| 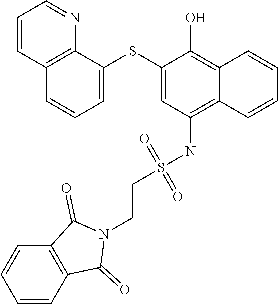 [SMILES: O=C1c2ccccc2C(=O)N1CCS(=O)(=O)Nc1cc(Sc2cccc3cccnc23)c(O)c2ccccc12] | C29H21N3O5S2 |
| 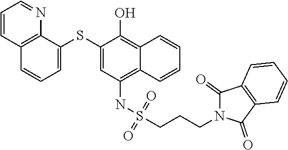 [SMILES: O=C1c2ccccc2C(=O)N1CCCS(=O)(=O)Nc1cc(Sc2cccc3cccnc23)c(O)c2ccccc12] | C30H23N3O5S2 |
| 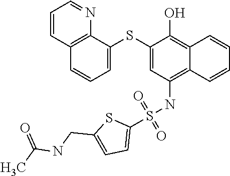 [SMILES: CC(=O)NCc1ccc(S(=O)(=O)Nc2cc(Sc3cccc4cccnc34)c(O)c3ccccc23)s1] | C26H21N3O4S3 |
| 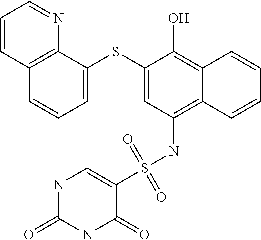 [SMILES: O=c1[nH]cc(S(=O)(=O)Nc2cc(Sc3cccc4cccnc34)c(O)c3ccccc23)c(=O)[nH]1] | C23H16N4O5S2 |
| 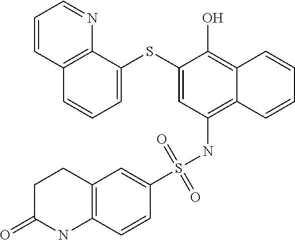 [SMILES: O=C1CCc2cc(S(=O)(=O)Nc3cc(Sc4cccc5cccnc45)c(O)c4ccccc34)ccc2N1] | C28H21N3O4S2 |
| 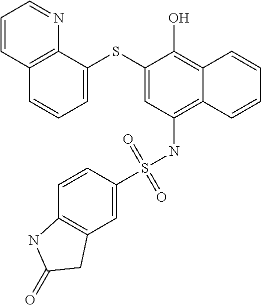 [SMILES: O=C1Cc2cc(S(=O)(=O)Nc3cc(Sc4cccc5cccnc45)c(O)c4ccccc34)ccc2N1] | C27H19N3O4S2 |
| 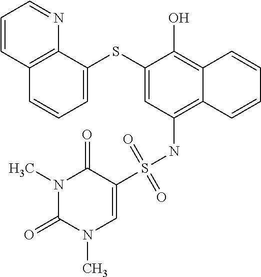 [SMILES: Cn1cc(S(=O)(=O)Nc2cc(Sc3cccc4cccnc34)c(O)c3ccccc23)c(=O)n(C)c1=O] | C25H20N4O5S2 |
| 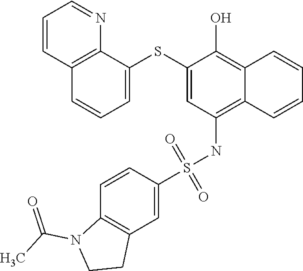 [SMILES: CC(=O)N1CCc2cc(S(=O)(=O)Nc3cc(Sc4cccc5cccnc45)c(O)c4ccccc34)ccc21] | C29H23N3O4S2 |
| 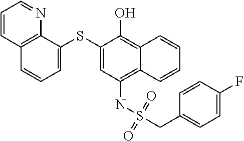 [SMILES: O=S(=O)(Cc1ccc(F)cc1)Nc1cc(Sc2cccc3cccnc23)c(O)c2ccccc12] | C26H19FN2O3S2 |
| 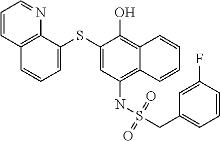 [SMILES: O=S(=O)(Cc1cccc(F)c1)Nc1cc(Sc2cccc3cccnc23)c(O)c2ccccc12] | C26H19FN2O3S2 |
| 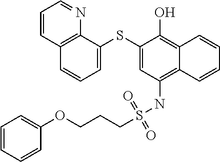 [SMILES: O=S(=O)(CCCOc1ccccc1)Nc1cc(Sc2cccc3cccnc23)c(O)c2ccccc12] | C28H24N2O4S2 |
| 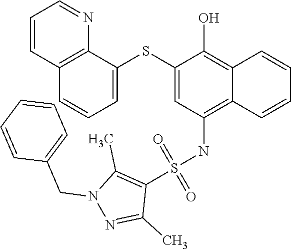 [SMILES: Cc1nn(Cc2ccccc2)c(C)c1S(=O)(=O)Nc1cc(Sc2cccc3cccnc23)c(O)c2ccccc12] | C31H26N4O3S2 |
| 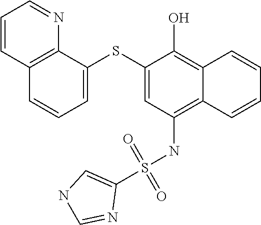 [SMILES: O=S(=O)(Nc1cc(Sc2cccc3cccnc23)c(O)c2ccccc12)c1c[nH]cn1] | C22H16N4O3S2 |
| 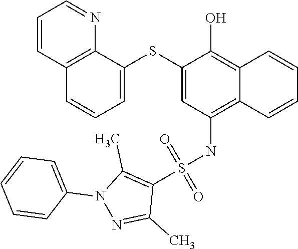 [SMILES: Cc1nn(-c2ccccc2)c(C)c1S(=O)(=O)Nc1cc(Sc2cccc3cccnc23)c(O)c2ccccc12] | C30H24N4O3S2 |
| 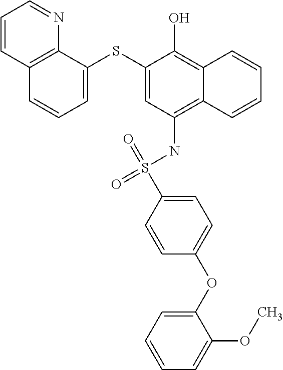 [SMILES: COc1ccccc1Oc1ccc(S(=O)(=O)Nc2cc(Sc3cccc4cccnc34)c(O)c3ccccc23)cc1] | C32H24N2O5S2 |
| 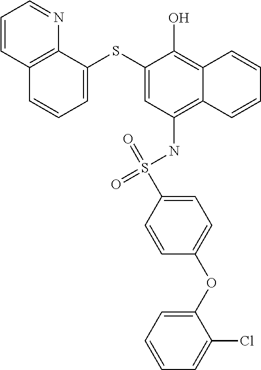 [SMILES: O=S(=O)(Nc1cc(Sc2cccc3cccnc23)c(O)c2ccccc12)c1ccc(Oc2ccccc2Cl)cc1] | C31H21ClN2O4S2 |
| 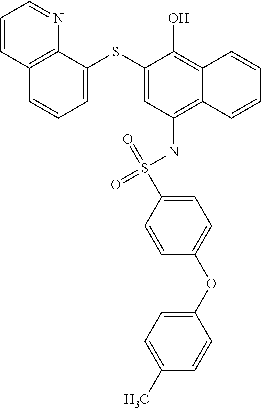 [SMILES: Cc1ccc(Oc2ccc(S(=O)(=O)Nc3cc(Sc4cccc5cccnc45)c(O)c4ccccc34)cc2)cc1] | C32H24N2O4S2 |
| 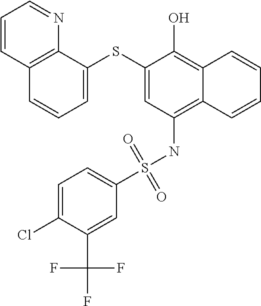 [SMILES: O=S(=O)(Nc1cc(Sc2cccc3cccnc23)c(O)c2ccccc12)c1ccc(Cl)c(C(F)(F)F)c1] | C26H16ClF3 N2O3S2 |
| 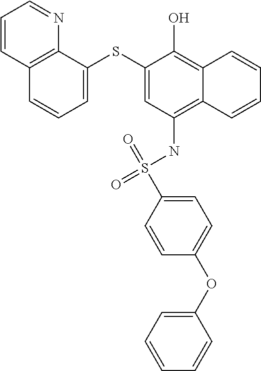 [SMILES: O=S(=O)(Nc1cc(Sc2cccc3cccnc23)c(O)c2ccccc12)c1ccc(Oc2ccccc2)cc1] | C31H22N2O4S2 |
| 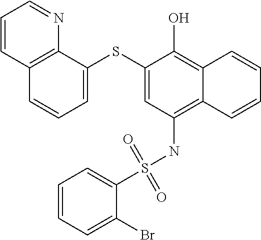 [SMILES: O=S(=O)(Nc1cc(Sc2cccc3cccnc23)c(O)c2ccccc12)c1ccccc1Br] | C25H17BrN2O3S2 |
| 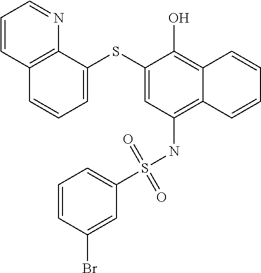 [SMILES: O=S(=O)(Nc1cc(Sc2cccc3cccnc23)c(O)c2ccccc12)c1cccc(Br)c1] | C25H17BrN2O3S2 |
| 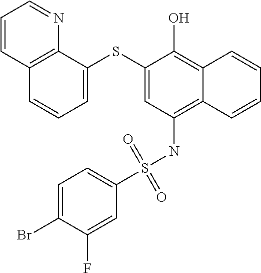 [SMILES: O=S(=O)(Nc1cc(Sc2cccc3cccnc23)c(O)c2ccccc12)c1ccc(Br)c(F)c1] | C25H16Br FN2O3S2 |
| 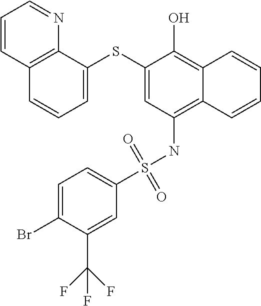 [SMILES: O=S(=O)(Nc1cc(Sc2cccc3cccnc23)c(O)c2ccccc12)c1ccc(Br)c(C(F)(F)F)c1] | C26H16Br F3N2O3S2 |
| 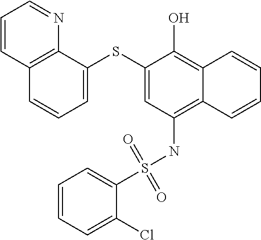 [SMILES: O=S(=O)(Nc1cc(Sc2cccc3cccnc23)c(O)c2ccccc12)c1ccccc1Cl] | C25H17ClN2O3S2 |
| 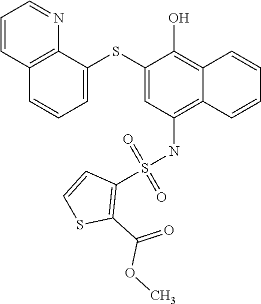 [SMILES: COC(=O)c1sccc1S(=O)(=O)Nc1cc(Sc2cccc3cccnc23)c(O)c2ccccc12] | C25H18N2O5S3 |
| 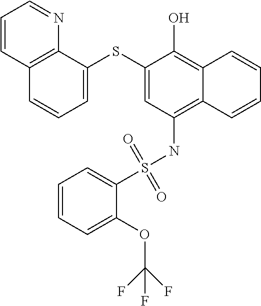 [SMILES: O=S(=O)(Nc1cc(Sc2cccc3cccnc23)c(O)c2ccccc12)c1ccccc1OC(F)(F)F] | C26H17F3N2O4S2 |

TABLE 5

| Structure | Formula structure |
|---|---|
| 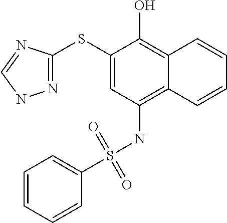 [SMILES: O=S(=O)(Nc1cc(Sc2nc[nH]n2)c(O)c2ccccc12)c1ccccc1] | C18H14N4O3S2 |
| 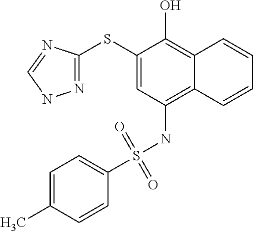 [SMILES: Cc1ccc(S(=O)(=O)Nc2cc(Sc3nc[nH]n3)c(O)c3ccccc23)cc1] | C19H16N4O3S2 |
| 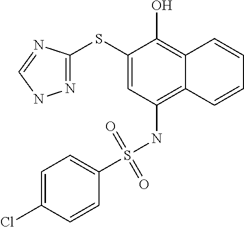 [SMILES: O=S(=O)(Nc1cc(Sc2nc[nH]n2)c(O)c2ccccc12)c1ccc(Cl)cc1] | C18H13ClN4O3S2 |
| 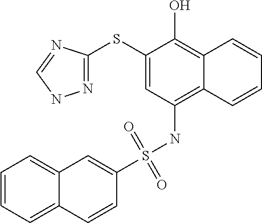 [SMILES: O=S(=O)(Nc1cc(Sc2nc[nH]n2)c(O)c2ccccc12)c1ccc2ccccc2c1] | C22H16N4O3S2 |
| 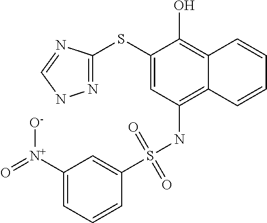 [SMILES: O=[N+]([O-])c1cccc(S(=O)(=O)Nc2cc(Sc3nc[nH]n3)c(O)c3ccccc23)c1] | C18H13N5O5S2 |
| 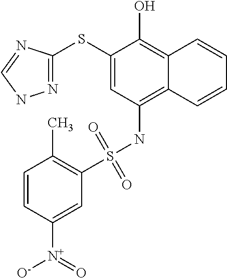 [SMILES: Cc1ccc([N+](=O)[O-])cc1S(=O)(=O)Nc1cc(Sc2nc[nH]n2)c(O)c2ccccc12] | C19H15N5O5S2 |
| 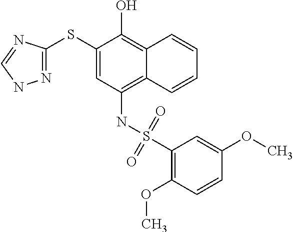 [SMILES: COc1ccc(OC)c(S(=O)(=O)Nc2cc(Sc3nc[nH]n3)c(O)c3ccccc23)c1] | C20H18N4O5S2 |
| 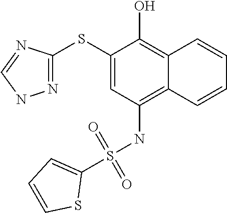 [SMILES: O=S(=O)(Nc1cc(Sc2nc[nH]n2)c(O)c2ccccc12)c1cccs1] | C16H12N4O3S3 |
| 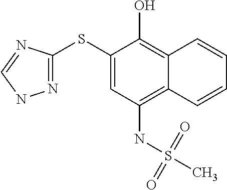 [SMILES: CS(=O)(=O)Nc1cc(Sc2nc[nH]n2)c(O)c2ccccc12] | C13H12N4O3S2 |
| 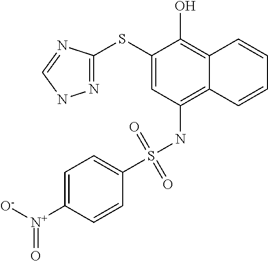 [SMILES: O=[N+]([O-])c1ccc(S(=O)(=O)Nc2cc(Sc3nc[nH]n3)c(O)c3ccccc23)cc1] | C18H13N5O5S2 |
| 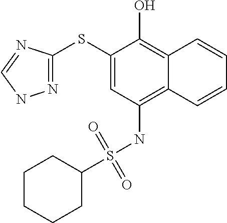 [SMILES: O=S(=O)(Nc1cc(Sc2nc[nH]n2)c(O)c2ccccc12)C1CCCCC1] | C18H20N4O3S2 |
| 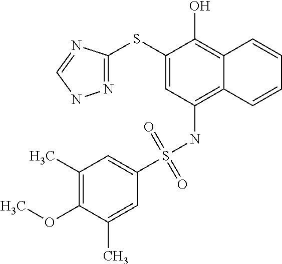 [SMILES: COc1c(C)cc(S(=O)(=O)Nc2cc(Sc3nc[nH]n3)c(O)c3ccccc23)cc1C] | C21H20N4O4S2 |
| 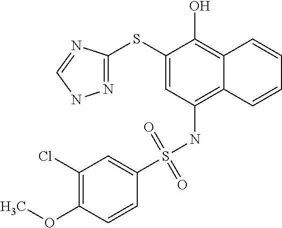 [SMILES: COc1ccc(S(=O)(=O)Nc2cc(Sc3nc[nH]n3)c(O)c3ccccc23)cc1Cl] | C19H15ClN4O4S2 |
| 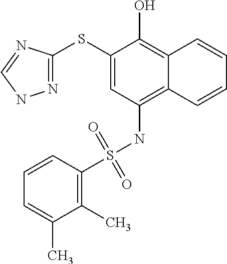 [SMILES: Cc1cccc(S(=O)(=O)Nc2cc(Sc3nc[nH]n3)c(O)c3ccccc23)c1C] | C20H18N4O3S2 |
| 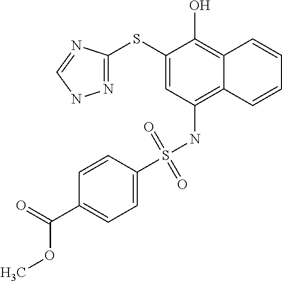 [SMILES: COC(=O)c1ccc(S(=O)(=O)Nc2cc(Sc3nc[nH]n3)c(O)c3ccccc23)cc1] | C20H16N4O5S2 |
| 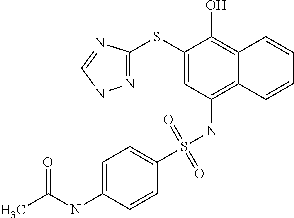 [SMILES: CC(=O)Nc1ccc(S(=O)(=O)Nc2cc(Sc3nc[nH]n3)c(O)c3ccccc23)cc1] | C20H17N5O4S2 |
| 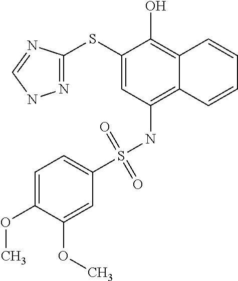 [SMILES: COc1ccc(S(=O)(=O)Nc2cc(Sc3nc[nH]n3)c(O)c3ccccc23)cc1OC] | C20H18N4O5S2 |
| 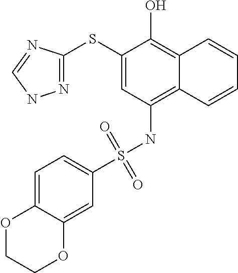 [SMILES: O=S(=O)(Nc1cc(Sc2nc[nH]n2)c(O)c2ccccc12)c1ccc2c(c1)OCCO2] | C20H16N4O5S2 |
| 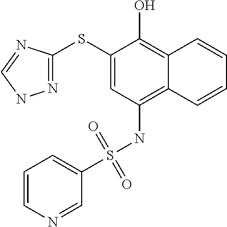 [SMILES: O=S(=O)(Nc1cc(Sc2nc[nH]n2)c(O)c2ccccc12)c1cccnc1] | C17H13N5O3S2 |
| 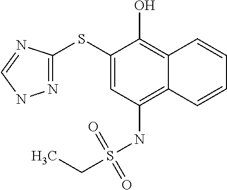 [SMILES: CCS(=O)(=O)Nc1cc(Sc2nc[nH]n2)c(O)c2ccccc12] | C14H14N4O3S2 |
| 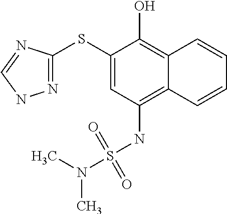 [SMILES: CN(C)S(=O)(=O)Nc1cc(Sc2nc[nH]n2)c(O)c2ccccc12] | C14H15N5O3S2 |
| 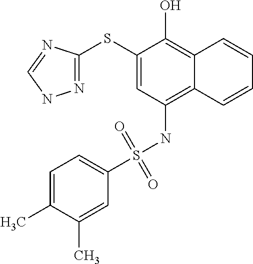 [SMILES: Cc1ccc(S(=O)(=O)Nc2cc(Sc3nc[nH]n3)c(O)c3ccccc23)cc1C] | C20H18N4O3S2 |
| 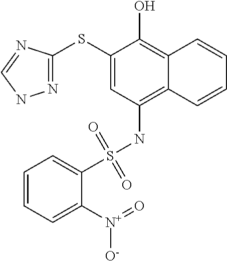 [SMILES: O=[N+]([O-])c1ccccc1S(=O)(=O)Nc1cc(Sc2nc[nH]n2)c(O)c2ccccc12] | C18H13N5O5S2 |
| 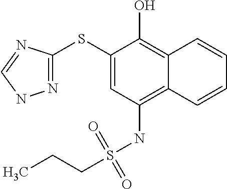 [SMILES: CCCS(=O)(=O)Nc1cc(Sc2nc[nH]n2)c(O)c2ccccc12] | C15H16N4O3S2 |
| 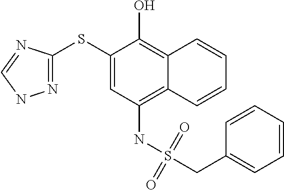 [SMILES: O=S(=O)(Cc1ccccc1)Nc1cc(Sc2nc[nH]n2)c(O)c2ccccc12] | C19H16N4O3S2 |
| 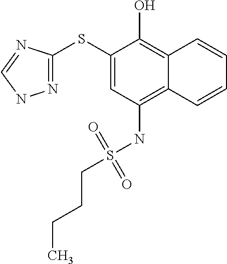 [SMILES: CCCCS(=O)(=O)Nc1cc(Sc2nc[nH]n2)c(O)c2ccccc12] | C16H18N4O3S2 |
| 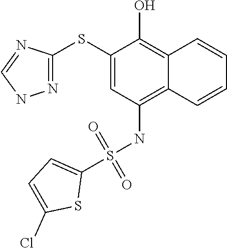 [SMILES: O=S(=O)(Nc1cc(Sc2nc[nH]n2)c(O)c2ccccc12)c1ccc(Cl)s1] | C16H11ClN4O3S3 |
| 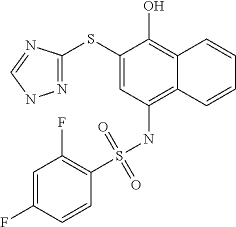 [SMILES: O=S(=O)(Nc1cc(Sc2nc[nH]n2)c(O)c2ccccc12)c1ccc(F)cc1F] | C18H12F2N4O3S2 |
| 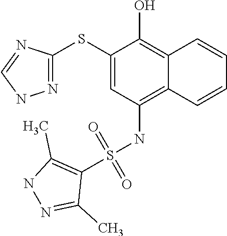 [SMILES: Cc1n[nH]c(C)c1S(=O)(=O)Nc1cc(Sc2nc[nH]n2)c(O)c2ccccc12] | C17H16N6O3S2 |
| 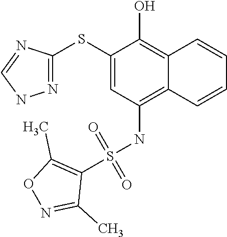 [SMILES: Cc1noc(C)c1S(=O)(=O)Nc1cc(Sc2nc[nH]n2)c(O)c2ccccc12] | C17H15N5O4S2 |
| 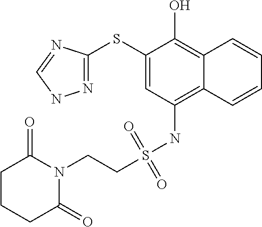 [SMILES: O=C1CCCC(=O)N1CCS(=O)(=O)Nc1cc(Sc2nc[nH]n2)c(O)c2ccccc12] | C19H19N5O5S2 |
| 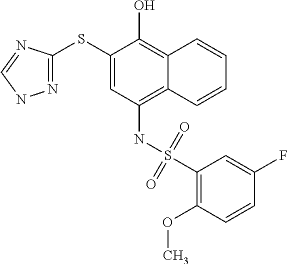 [SMILES: COc1ccc(F)cc1S(=O)(=O)Nc1cc(Sc2nc[nH]n2)c(O)c2ccccc12] | C19H15FN4O4S2 |
| 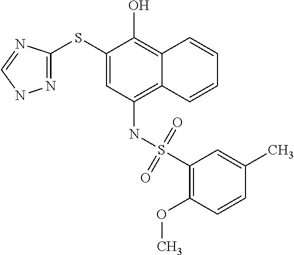 [SMILES: COc1ccc(C)cc1S(=O)(=O)Nc1cc(Sc2nc[nH]n2)c(O)c2ccccc12] | C20H18N4O4S2 |
| 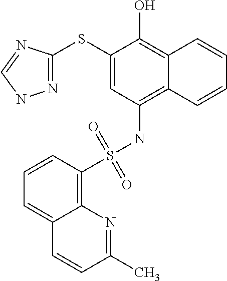 [SMILES: Cc1ccc2cccc(S(=O)(=O)Nc3cc(Sc4nc[nH]n4)c(O)c4ccccc34)c2n1] | C22H17N5O3S2 |
| 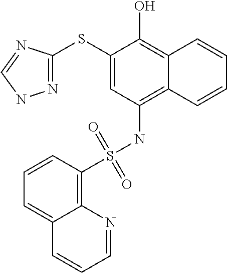 [SMILES: O=S(=O)(Nc1cc(Sc2nc[nH]n2)c(O)c2ccccc12)c1cccc2cccnc12] | C21H15N5O3S2 |
| 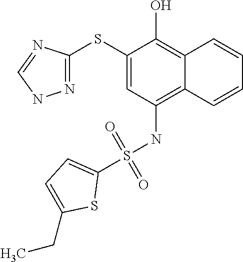 [SMILES: CCc1ccc(S(=O)(=O)Nc2cc(Sc3nc[nH]n3)c(O)c3ccccc23)s1] | C18H16N4O3S3 |
| 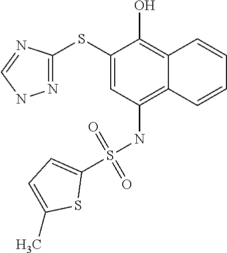 [SMILES: Cc1ccc(S(=O)(=O)Nc2cc(Sc3nc[nH]n3)c(O)c3ccccc23)s1] | C17H14N4O3S3 |
| 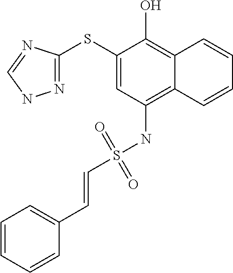 [SMILES: O=S(=O)(/C=C/c1ccccc1)Nc1cc(Sc2nc[nH]n2)c(O)c2ccccc12] | C20H16N4O3S2 |
| 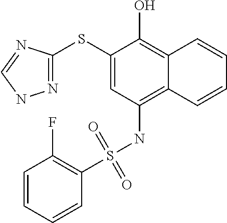 [SMILES: O=S(=O)(Nc1cc(Sc2nc[nH]n2)c(O)c2ccccc12)c1ccccc1F] | C18H13FN4O3S2 |
| 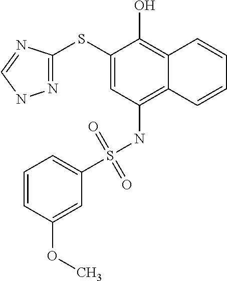 [SMILES: COc1cccc(S(=O)(=O)Nc2cc(Sc3nc[nH]n3)c(O)c3ccccc23)c1] | C19H16N4O4S2 |
| 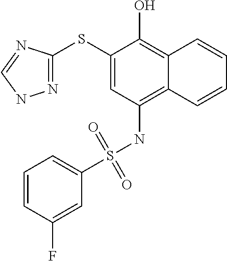 [SMILES: O=S(=O)(Nc1cc(Sc2nc[nH]n2)c(O)c2ccccc12)c1cccc(F)c1] | C18H13FN4O3S2 |
| 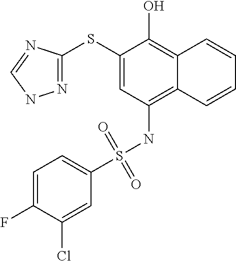 [SMILES: O=S(=O)(Nc1cc(Sc2nc[nH]n2)c(O)c2ccccc12)c1ccc(F)c(Cl)c1] | C18H12ClFN4O3S2 |
| 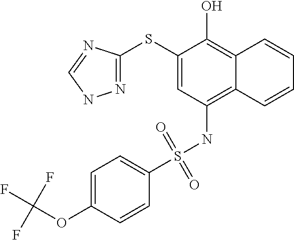 [SMILES: O=S(=O)(Nc1cc(Sc2nc[nH]n2)c(O)c2ccccc12)c1ccc(OC(F)(F)F)cc1] | C19H13F3N4O4S2 |
| 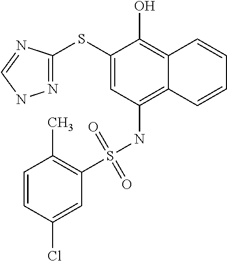 [SMILES: Cc1ccc(Cl)cc1S(=O)(=O)Nc1cc(Sc2nc[nH]n2)c(O)c2ccccc12] | C19H15ClN4O3S2 |
| 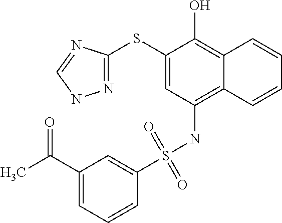 [SMILES: CC(=O)c1cccc(S(=O)(=O)Nc2cc(Sc3nc[nH]n3)c(O)c3ccccc23)c1] | C20H16N4O4S2 |
| 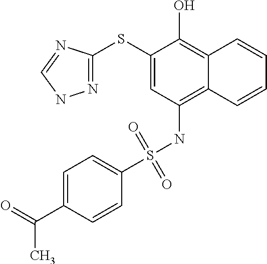 [SMILES: CC(=O)c1ccc(S(=O)(=O)Nc2cc(Sc3nc[nH]n3)c(O)c3ccccc23)cc1] | C20H16N4O4S2 |
| 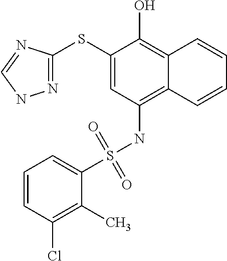 [SMILES: Cc1c(Cl)cccc1S(=O)(=O)Nc1cc(Sc2nc[nH]n2)c(O)c2ccccc12] | C19H15ClN4O3S2 |
| 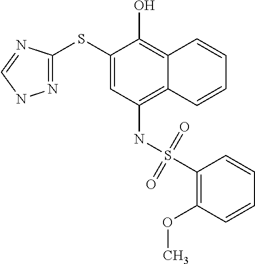 [SMILES: COc1ccccc1S(=O)(=O)Nc1cc(Sc2nc[nH]n2)c(O)c2ccccc12] | C19H16N4O4S2 |
| 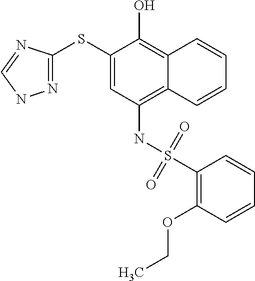 [SMILES: CCOc1ccccc1S(=O)(=O)Nc1cc(Sc2nc[nH]n2)c(O)c2ccccc12] | C20H18N4O4S2 |
| 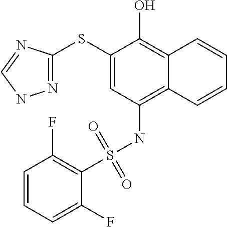 [SMILES: O=S(=O)(Nc1cc(Sc2nc[nH]n2)c(O)c2ccccc12)c1c(F)cccc1F] | C18H12F2N4O3S2 |
| 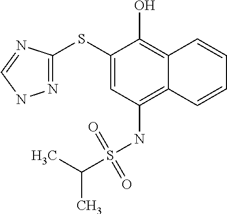 [SMILES: CC(C)S(=O)(=O)Nc1cc(Sc2nc[nH]n2)c(O)c2ccccc12] | C15H16N4O3S2 |
| 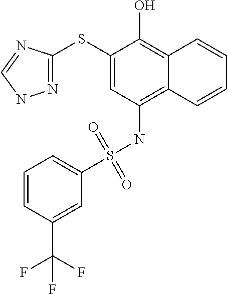 [SMILES: O=S(=O)(Nc1cc(Sc2nc[nH]n2)c(O)c2ccccc12)c1cccc(C(F)(F)F)c1] | C19H13F3N4O3S2 |
| 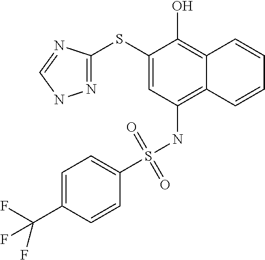 [SMILES: O=S(=O)(Nc1cc(Sc2nc[nH]n2)c(O)c2ccccc12)c1ccc(C(F)(F)F)cc1] | C19H13F3N4O3S2 |
| 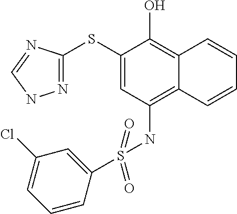 [SMILES: O=S(=O)(Nc1cc(Sc2nc[nH]n2)c(O)c2ccccc12)c1cccc(Cl)c1] | C18H13ClN4O3S2 |
| 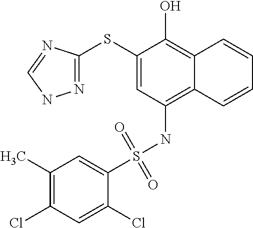 [SMILES: Cc1cc(S(=O)(=O)Nc2cc(Sc3nc[nH]n3)c(O)c3ccccc23)c(Cl)cc1Cl] | C19H14Cl2N4O3S2 |
| 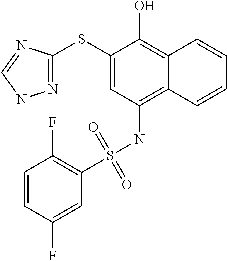 [SMILES: O=S(=O)(Nc1cc(Sc2nc[nH]n2)c(O)c2ccccc12)c1cc(F)ccc1F] | C18H12F2N4O3S2 |
| 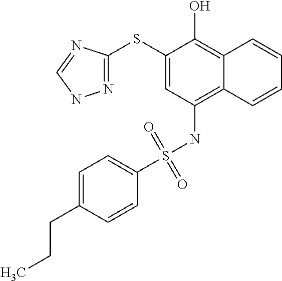 [SMILES: CCCc1ccc(S(=O)(=O)Nc2cc(Sc3nc[nH]n3)c(O)c3ccccc23)cc1] | C21H20N4O3S2 |
| 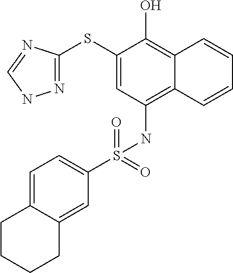 [SMILES: O=S(=O)(Nc1cc(Sc2nc[nH]n2)c(O)c2ccccc12)c1ccc2c(c1)CCCC2] | C22H20N4O3S2 |
| 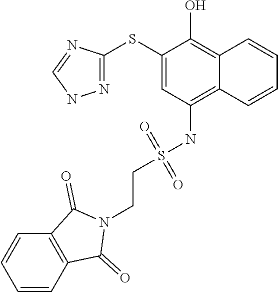 [SMILES: O=C1c2ccccc2C(=O)N1CCS(=O)(=O)Nc1cc(Sc2nc[nH]n2)c(O)c2ccccc12] | C22H17N5O5S2 |
| 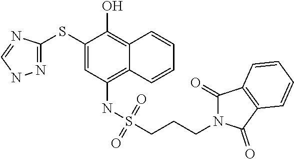 [SMILES: O=C1c2ccccc2C(=O)N1CCCS(=O)(=O)Nc1cc(Sc2nc[nH]n2)c(O)c2ccccc12] | C23H19N5O5S2 |
| 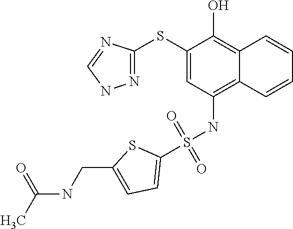 [SMILES: CC(=O)NCc1ccc(S(=O)(=O)Nc2cc(Sc3nc[nH]n3)c(O)c3ccccc23)s1] | C19H17N5O4S3 |
| 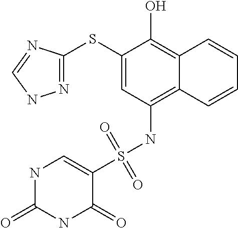 [SMILES: O=c1[nH]cc(S(=O)(=O)Nc2cc(Sc3nc[nH]n3)c(O)c3ccccc23)c(=O)[nH]1] | C16H12N6O5S2 |
| 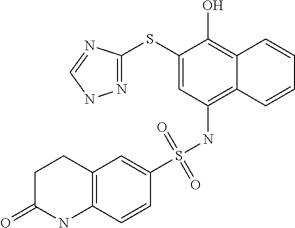 [SMILES: O=C1CCc2cc(S(=O)(=O)Nc3cc(Sc4nc[nH]n4)c(O)c4ccccc34)ccc2N1] | C21H17N5O4S2 |
| 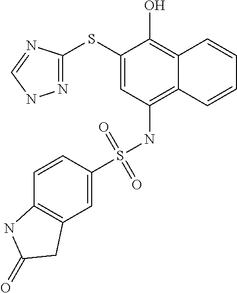 [SMILES: O=C1Cc2cc(S(=O)(=O)Nc3cc(Sc4nc[nH]n4)c(O)c4ccccc34)ccc2N1] | C20H15N5O4S2 |
| 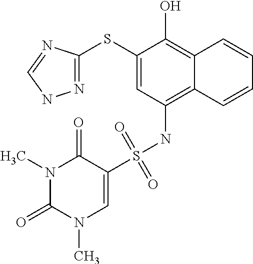 [SMILES: Cn1cc(S(=O)(=O)Nc2cc(Sc3nc[nH]n3)c(O)c3ccccc23)c(=O)n(C)c1=O] | C18H16N6O5S2 |
| 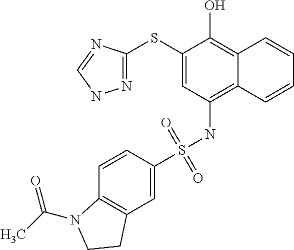 [SMILES: CC(=O)N1CCc2cc(S(=O)(=O)Nc3cc(Sc4nc[nH]n4)c(O)c4ccccc34)ccc21] | C22H19N5O4S2 |
| 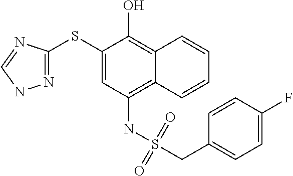 [SMILES: O=S(=O)(Cc1ccc(F)cc1)Nc1cc(Sc2nc[nH]n2)c(O)c2ccccc12] | C19H15FN4O3S2 |
| 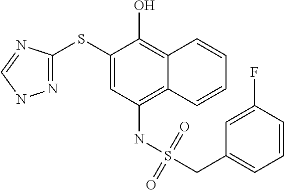 [SMILES: O=S(=O)(Cc1cccc(F)c1)Nc1cc(Sc2nc[nH]n2)c(O)c2ccccc12] | C19H15FN4O3S2 |
| 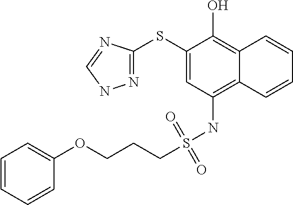 [SMILES: O=S(=O)(CCCOc1ccccc1)Nc1cc(Sc2nc[nH]n2)c(O)c2ccccc12] | C21H20N4O4S2 |
| 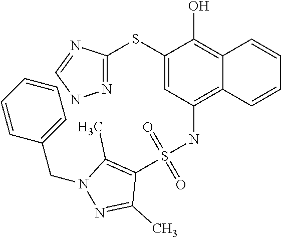 [SMILES: Cc1nn(Cc2ccccc2)c(C)c1S(=O)(=O)Nc1cc(Sc2nc[nH]n2)c(O)c2ccccc12] | C24H22N6O3S2 |
| 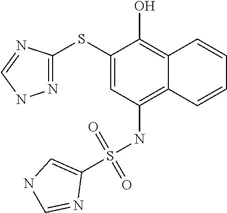 [SMILES: O=S(=O)(Nc1cc(Sc2nc[nH]n2)c(O)c2ccccc12)c1c[nH]cn1] | C15H12N603S2 |
| 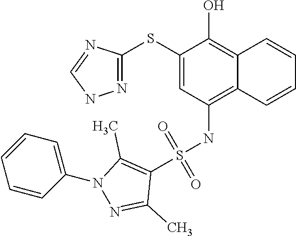 [SMILES: Cc1nn(-c2ccccc2)c(C)c1S(=O)(=O)Nc1cc(Sc2nc[nH]n2)c(O)c2ccccc12] | C23H20N6O3S2 |
| 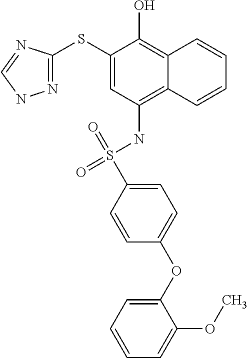 [SMILES: COc1ccccc1Oc1ccc(S(=O)(=O)Nc2cc(Sc3nc[nH]n3)c(O)c3ccccc23)cc1] | C25H20N4O5S2 |
| 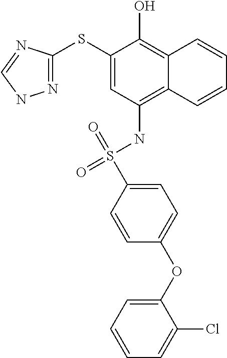 [SMILES: O=S(=O)(Nc1cc(Sc2nc[nH]n2)c(O)c2ccccc12)c1ccc(Oc2ccccc2Cl)cc1] | C24H17ClN4O4S2 |
| 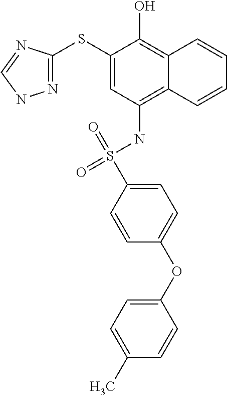 [SMILES: Cc1ccc(Oc2ccc(S(=O)(=O)Nc3cc(Sc4nc[nH]n4)c(O)c4ccccc34)cc2)cc1] | C25H20N4O4S2 |
| 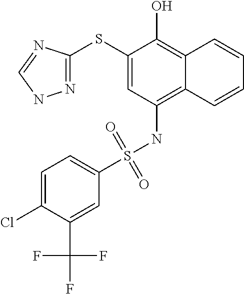 [SMILES: O=S(=O)(Nc1cc(Sc2nc[nH]n2)c(O)c2ccccc12)c1ccc(Cl)c(C(F)(F)F)c1] | C19H12ClF3N4O3S2 |
| 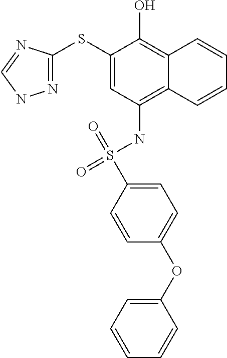 [SMILES: O=S(=O)(Nc1cc(Sc2nc[nH]n2)c(O)c2ccccc12)c1ccc(Oc2ccccc2)cc1] | C24H18N4O4S2 |
| 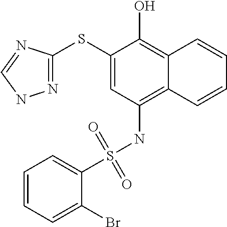 [SMILES: O=S(=O)(Nc1cc(Sc2nc[nH]n2)c(O)c2ccccc12)c1ccccc1Br] | C18H13BrN4O3S2 |
| 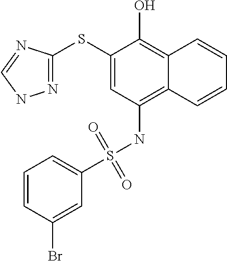 [SMILES: O=S(=O)(Nc1cc(Sc2nc[nH]n2)c(O)c2ccccc12)c1cccc(Br)c1] | C18H13BrN4O3S2 |
| 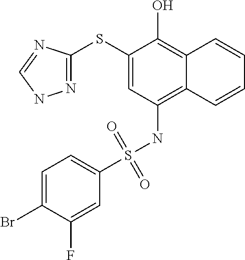 [SMILES: O=S(=O)(Nc1cc(Sc2nc[nH]n2)c(O)c2ccccc12)c1ccc(Br)c(F)c1] | C18H12BrFN4O3S2 |
| 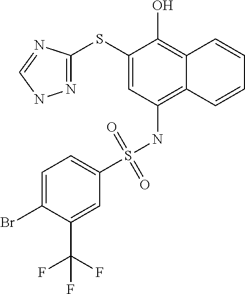 [SMILES: O=S(=O)(Nc1cc(Sc2nc[nH]n2)c(O)c2ccccc12)c1ccc(Br)c(C(F)(F)F)c1] | C19H12BrF3N4O3S2 |
| 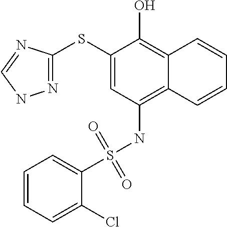 [SMILES: O=S(=O)(Nc1cc(Sc2nc[nH]n2)c(O)c2ccccc12)c1ccccc1Cl] | C18H13ClN4O3S2 |
| 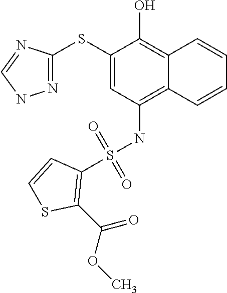 [SMILES: COC(=O)c1sccc1S(=O)(=O)Nc1cc(Sc2nc[nH]n2)c(O)c2ccccc12] | C18H14N4O5S3 |
| 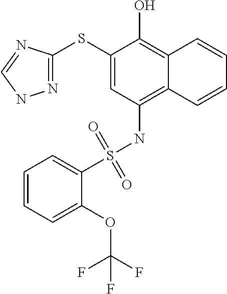 [SMILES: O=S(=O)(Nc1cc(Sc2nc[nH]n2)c(O)c2ccccc12)c1ccccc1OC(F)(F)F] | C19H13F3N4O4S2 |

TABLE 6

| Structure | Formula structure |
|---|---|
| 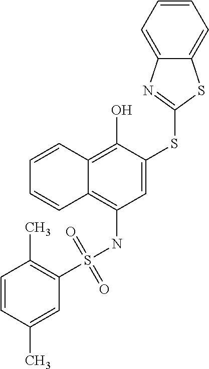 [SMILES: Cc1ccc(C)c(S(=O)(=O)Nc2cc(Sc3nc4ccccc4s3)c(O)c3ccccc23)c1] | C25H20N2O3S3 |
| 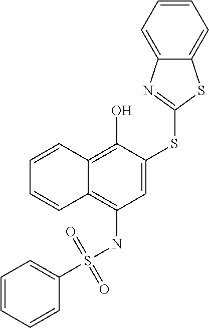 [SMILES: O=S(=O)(Nc1cc(Sc2nc3ccccc3s2)c(O)c2ccccc12)c1ccccc1] | C23H16N2O3S3 |
| 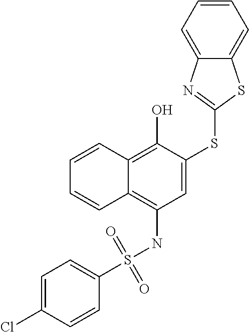 [SMILES: O=S(=O)(Nc1cc(Sc2nc3ccccc3s2)c(O)c2ccccc12)c1ccc(Cl)cc1] | C23H15ClN2O3S3 |
| 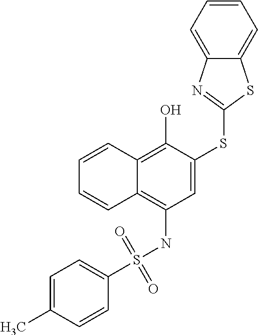 [SMILES: Cc1ccc(S(=O)(=O)Nc2cc(Sc3nc4ccccc4s3)c(O)c3ccccc23)cc1] | C24H18N2O3S3 |
| 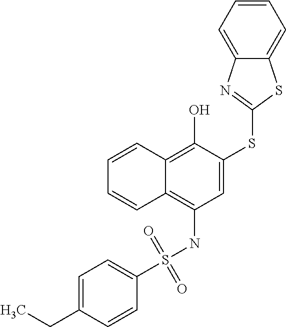 [SMILES: CCc1ccc(S(=O)(=O)Nc2cc(Sc3nc4ccccc4s3)c(O)c3ccccc23)cc1] | C25H20N2O3S3 |
| 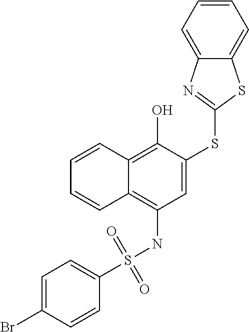 [SMILES: O=S(=O)(Nc1cc(Sc2nc3ccccc3s2)c(O)c2ccccc12)c1ccc(Br)cc1] | C23H15BrN2O3S3 |
| 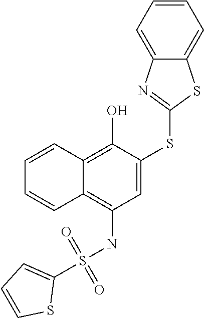 [SMILES: O=S(=O)(Nc1cc(Sc2nc3ccccc3s2)c(O)c2ccccc12)c1cccs1] | C21H14N2O3S4 |
| 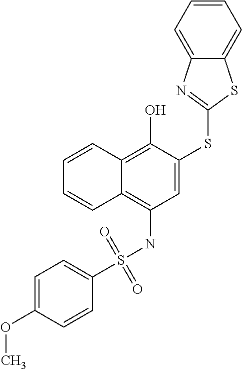 [SMILES: COc1ccc(S(=O)(=O)Nc2cc(Sc3nc4ccccc4s3)c(O)c3ccccc23)cc1] | C24H18N2O4S3 |
| 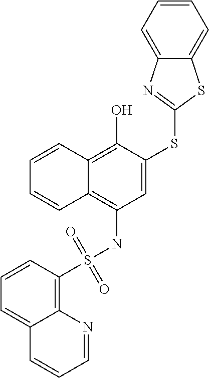 [SMILES: O=S(=O)(Nc1cc(Sc2nc3ccccc3s2)c(O)c2ccccc12)c1cccc2cccnc12] | C26H17N3O3S3 |
| 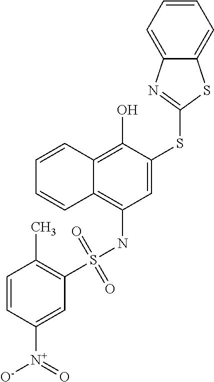 [SMILES: Cc1ccc([N+](=O)[O-])cc1S(=O)(=O)Nc1cc(Sc2nc3ccccc3s2)c(O)c2ccccc12] | C24H17N3O5S3 |
| 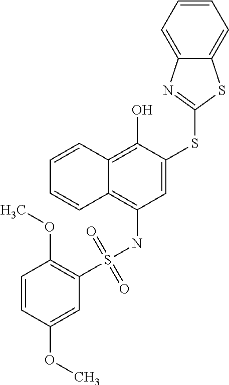 [SMILES: COc1ccc(OC)c(S(=O)(=O)Nc2cc(Sc3nc4ccccc4s3)c(O)c3ccccc23)c1] | C25H20N2O5S3 |
| 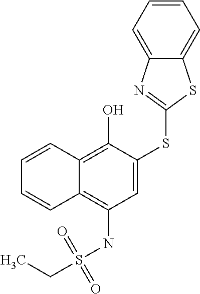 [SMILES: CCS(=O)(=O)Nc1cc(Sc2nc3ccccc3s2)c(O)c2ccccc12] | C19H16N2O3S3 |
| 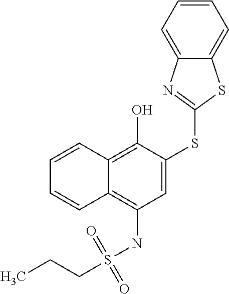 [SMILES: CCCS(=O)(=O)Nc1cc(Sc2nc3ccccc3s2)c(O)c2ccccc12] | C20H18N2O3S3 |
| 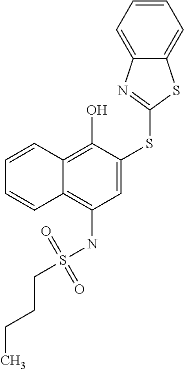 [SMILES: CCCCS(=O)(=O)Nc1cc(Sc2nc3ccccc3s2)c(O)c2ccccc12] | C21H20N2O3S3 |
| 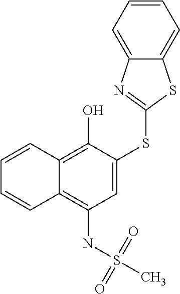 [SMILES: CS(=O)(=O)Nc1cc(Sc2nc3ccccc3s2)c(O)c2ccccc12] | C18H14N2O3S3 |
| 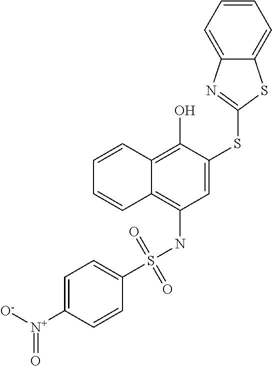 [SMILES: O=[N+]([O-])c1ccc(S(=O)(=O)Nc2cc(Sc3nc4ccccc4s3)c(O)c3ccccc23)cc1] | C23H15N3O5S3 |
| 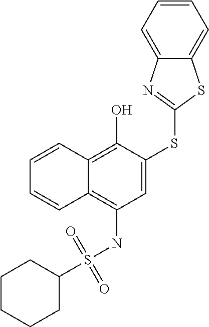 [SMILES: O=S(=O)(Nc1cc(Sc2nc3ccccc3s2)c(O)c2ccccc12)C1CCCCC1] | C23H22N2O3S3 |
| 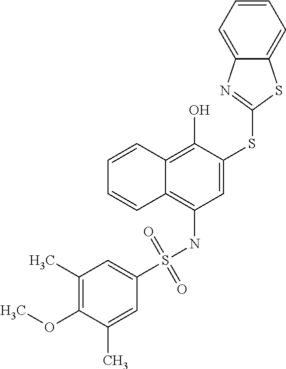 [SMILES: COc1c(C)cc(S(=O)(=O)Nc2cc(Sc3nc4ccccc4s3)c(O)c3ccccc23)cc1C] | C26H22N2O4S3 |
| 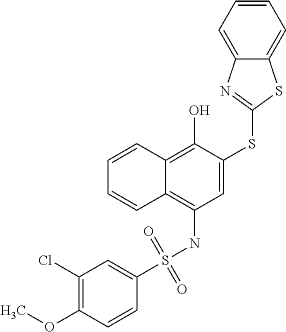 [SMILES: COc1ccc(S(=O)(=O)Nc2cc(Sc3nc4ccccc4s3)c(O)c3ccccc23)cc1Cl] | C24H17ClN2O4S3 |
| 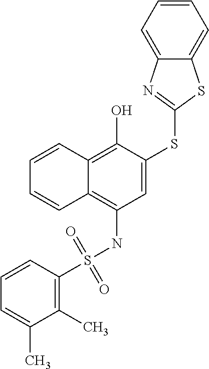 [SMILES: Cc1cccc(S(=O)(=O)Nc2cc(Sc3nc4ccccc4s3)c(O)c3ccccc23)c1C] | C25H20N2O3S3 |
| 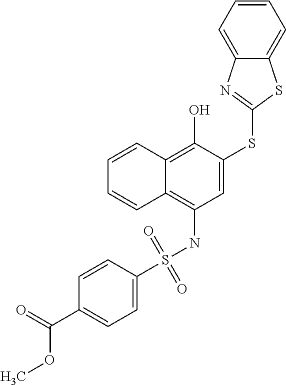 [SMILES: COC(=O)c1ccc(S(=O)(=O)Nc2cc(Sc3nc4ccccc4s3)c(O)c3ccccc23)cc1] | C25H18N2O5S3 |
| 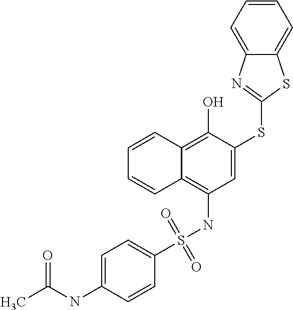 [SMILES: CC(=O)Nc1ccc(S(=O)(=O)Nc2cc(Sc3nc4ccccc4s3)c(O)c3ccccc23)cc1] | C25H19N3O4S3 |
| 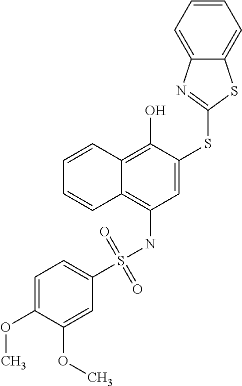 [SMILES: COc1ccc(S(=O)(=O)Nc2cc(Sc3nc4ccccc4s3)c(O)c3ccccc23)cc1OC] | C25H20N2O5S3 |
| 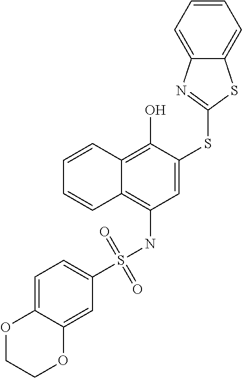 [SMILES: O=S(=O)(Nc1cc(Sc2nc3ccccc3s2)c(O)c2ccccc12)c1ccc2c(c1)OCCO2] | C25H18N2O5S3 |
| 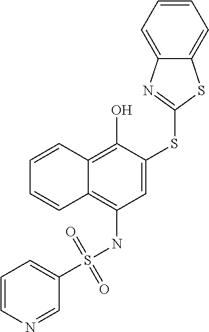 [SMILES: O=S(=O)(Nc1cc(Sc2nc3ccccc3s2)c(O)c2ccccc12)c1cccnc1] | C22H15N3O3S3 |
| 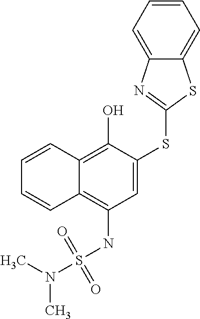 [SMILES: CN(C)S(=O)(=O)Nc1cc(Sc2nc3ccccc3s2)c(O)c2ccccc12] | C19H17N3O3S3 |
| 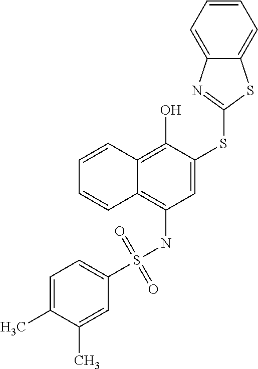 [SMILES: Cc1ccc(S(=O)(=O)Nc2cc(Sc3nc4ccccc4s3)c(O)c3ccccc23)cc1C] | C25H20N2O3S3 |
| 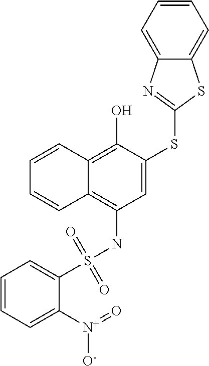 [SMILES: O=[N+]([O-])c1ccccc1S(=O)(=O)Nc1cc(Sc2nc3ccccc3s2)c(O)c2ccccc12] | C23H15N3O5S3 |
| 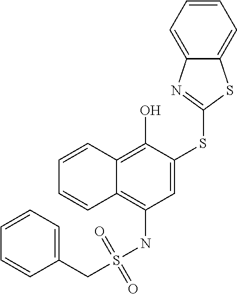 [SMILES: O=S(=O)(Cc1ccccc1)Nc1cc(Sc2nc3ccccc3s2)c(O)c2ccccc12] | C24H18N2O3S3 |
| 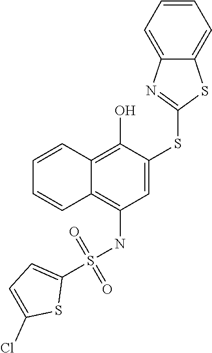 [SMILES: O=S(=O)(Nc1cc(Sc2nc3ccccc3s2)c(O)c2ccccc12)c1ccc(Cl)s1] | C21H13ClN2O3S4 |
| 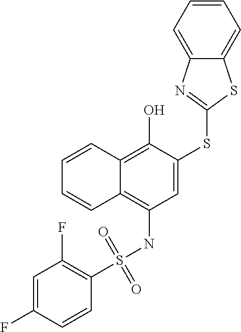 [SMILES: O=S(=O)(Nc1cc(Sc2nc3ccccc3s2)c(O)c2ccccc12)c1ccc(F)cc1F] | C23H14F2N2O3S3 |
| 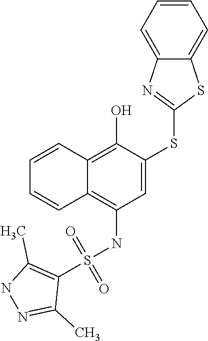 [SMILES: Cc1n[nH]c(C)c1S(=O)(=O)Nc1cc(Sc2nc3ccccc3s2)c(O)c2ccccc12] | C22H18N4O3S3 |
| 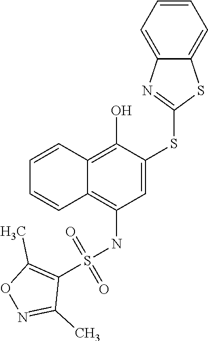 [SMILES: Cc1noc(C)c1S(=O)(=O)Nc1cc(Sc2nc3ccccc3s2)c(O)c2ccccc12] | C22H17N3O4S3 |
| 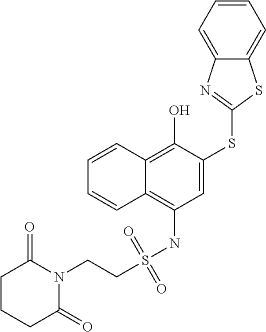 [SMILES: O=C1CCCC(=O)N1CCS(=O)(=O)Nc1cc(Sc2nc3ccccc3s2)c(O)c2ccccc12] | C24H21N3O5S3 |
| 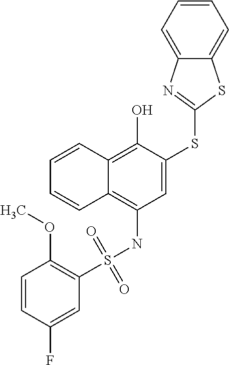 [SMILES: COc1ccc(F)cc1S(=O)(=O)Nc1cc(Sc2nc3ccccc3s2)c(O)c2ccccc12] | C24H17FN2O4S3 |
| 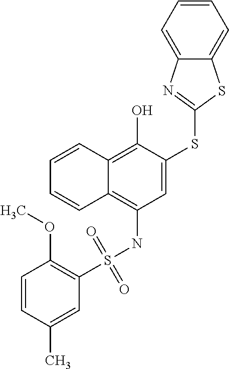 [SMILES: COc1ccc(C)cc1S(=O)(=O)Nc1cc(Sc2nc3ccccc3s2)c(O)c2ccccc12] | C25H20N2O4S3 |
| 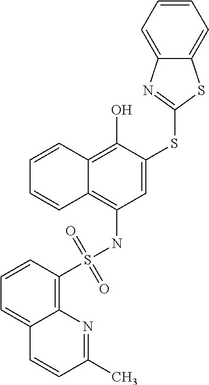 [SMILES: Cc1ccc2cccc(S(=O)(=O)Nc3cc(Sc4nc5ccccc5s4)c(O)c4ccccc34)c2n1] | C27H19N3O3S3 |
| 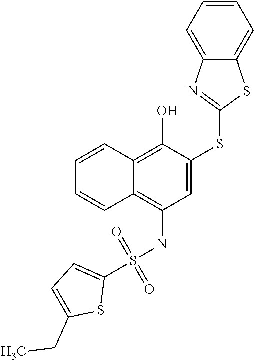 [SMILES: CCc1ccc(S(=O)(=O)Nc2cc(Sc3nc4ccccc4s3)c(O)c3ccccc23)s1] | C23H18N2O3S4 |
| 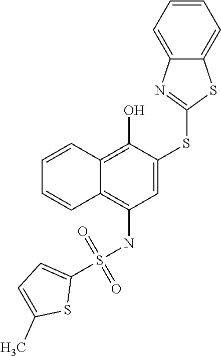 [SMILES: Cc1ccc(S(=O)(=O)Nc2cc(Sc3nc4ccccc4s3)c(O)c3ccccc23)s1] | C22H16N2O3S4 |
| 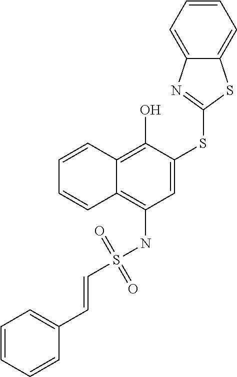 [SMILES: O=S(=O)(/C=C/c1ccccc1)Nc1cc(Sc2nc3ccccc3s2)c(O)c2ccccc12] | C25H18N2O3S3 |
| 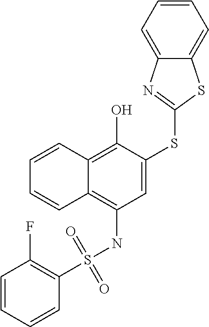 [SMILES: O=S(=O)(Nc1cc(Sc2nc3ccccc3s2)c(O)c2ccccc12)c1ccccc1F] | C23H15FN2O3S3 |
| 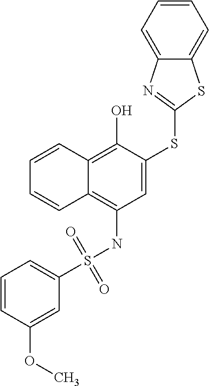 [SMILES: COc1cccc(S(=O)(=O)Nc2cc(Sc3nc4ccccc4s3)c(O)c3ccccc23)c1] | C24H18N2O4S3 |
| 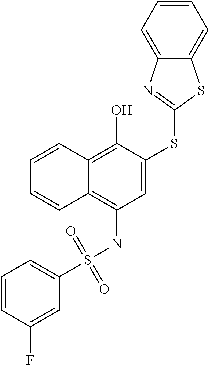 [SMILES: O=S(=O)(Nc1cc(Sc2nc3ccccc3s2)c(O)c2ccccc12)c1cccc(F)c1] | C23H15FN2O3S3 |
| 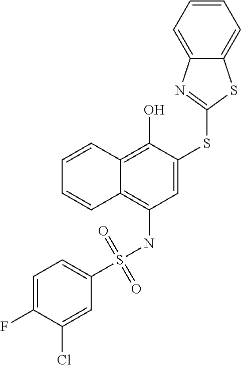 [SMILES: O=S(=O)(Nc1cc(Sc2nc3ccccc3s2)c(O)c2ccccc12)c1ccc(F)c(Cl)c1] | C23H14ClFN2O3S3 |
| 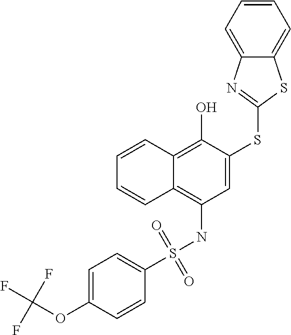 [SMILES: O=S(=O)(Nc1cc(Sc2nc3ccccc3s2)c(O)c2ccccc12)c1ccc(OC(F)(F)F)cc1] | C24H15F3N2O4S3 |
| 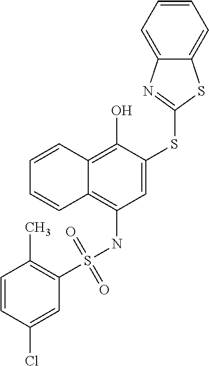 [SMILES: Cc1ccc(Cl)cc1S(=O)(=O)Nc1cc(Sc2nc3ccccc3s2)c(O)c2ccccc12] | C24H17ClN2O3S3 |
| 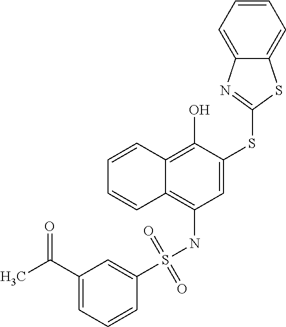 [SMILES: CC(=O)c1cccc(S(=O)(=O)Nc2cc(Sc3nc4ccccc4s3)c(O)c3ccccc23)c1] | C25H18N2O4S3 |
| 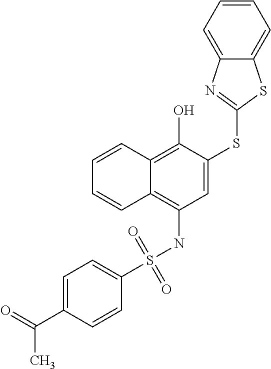 [SMILES: CC(=O)c1ccc(S(=O)(=O)Nc2cc(Sc3nc4ccccc4s3)c(O)c3ccccc23)cc1] | C25H18N2O4S3 |
| 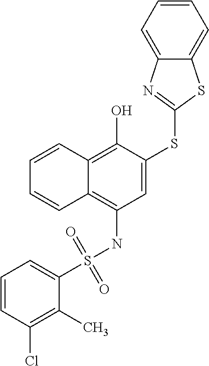 [SMILES: Cc1c(Cl)cccc1S(=O)(=O)Nc1cc(Sc2nc3ccccc3s2)c(O)c2ccccc12] | C24H17ClN2O3S3 |
| 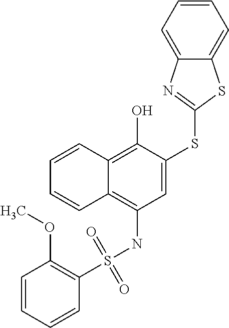 [SMILES: COc1ccccc1S(=O)(=O)Nc1cc(Sc2nc3ccccc3s2)c(O)c2ccccc12] | C24H18N2O4S3 |
| 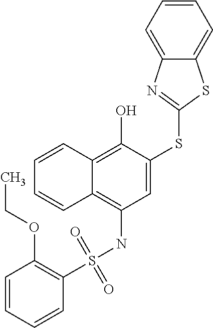 [SMILES: CCOc1ccccc1S(=O)(=O)Nc1cc(Sc2nc3ccccc3s2)c(O)c2ccccc12] | C25H20N2O4S3 |
| 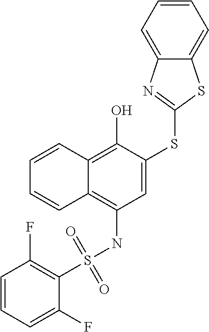 [SMILES: O=S(=O)(Nc1cc(Sc2nc3ccccc3s2)c(O)c2ccccc12)c1c(F)cccc1F] | C23H14F2N2O3S3 |
| 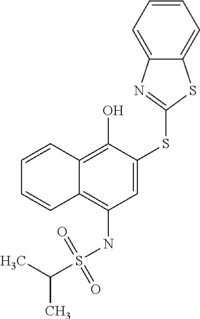 [SMILES: CC(C)S(=O)(=O)Nc1cc(Sc2nc3ccccc3s2)c(O)c2ccccc12] | C20H18N2O3S3 |
| 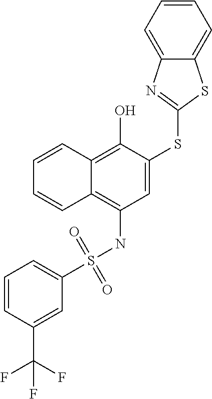 [SMILES: O=S(=O)(Nc1cc(Sc2nc3ccccc3s2)c(O)c2ccccc12)c1cccc(C(F)(F)F)c1] | C24H15F3N2O3S3 |
| 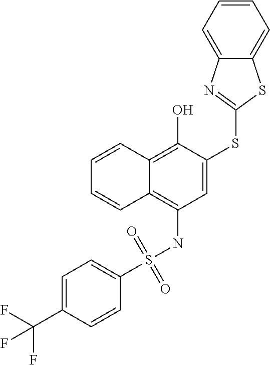 [SMILES: O=S(=O)(Nc1cc(Sc2nc3ccccc3s2)c(O)c2ccccc12)c1ccc(C(F)(F)F)cc1] | C24H15F3N2O3S3 |
| 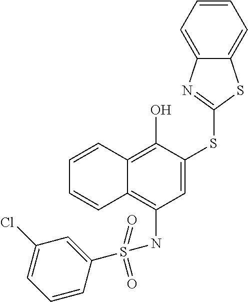 [SMILES: O=S(=O)(Nc1cc(Sc2nc3ccccc3s2)c(O)c2ccccc12)c1cccc(Cl)c1] | C23H15ClN2O3S3 |
| 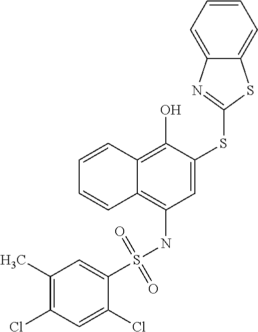 [SMILES: Cc1cc(S(=O)(=O)Nc2cc(Sc3nc4ccccc4s3)c(O)c3ccccc23)c(Cl)cc1Cl] | C24H16Cl2N2O3S3 |
| 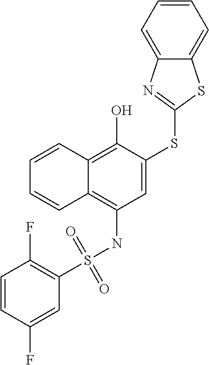 [SMILES: O=S(=O)(Nc1cc(Sc2nc3ccccc3s2)c(O)c2ccccc12)c1cc(F)ccc1F] | C23H14F2N2O3S3 |
| 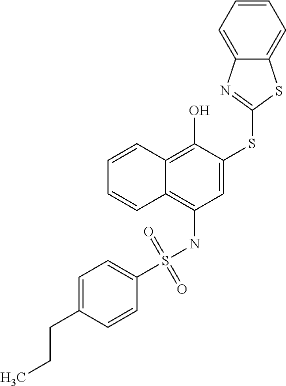 [SMILES: CCCc1ccc(S(=O)(=O)Nc2cc(Sc3nc4ccccc4s3)c(O)c3ccccc23)cc1] | C26H22N2O3S3 |
| 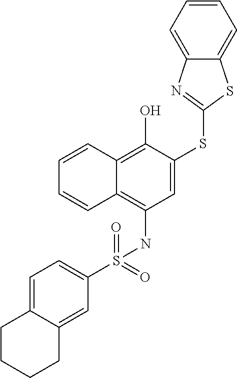 [SMILES: O=S(=O)(Nc1cc(Sc2nc3ccccc3s2)c(O)c2ccccc12)c1ccc2c(c1)CCCC2] | C27H22N2O3S3 |
| 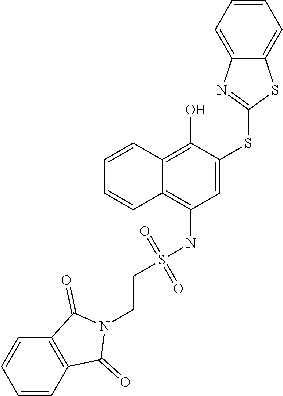 [SMILES: O=C1c2ccccc2C(=O)N1CCS(=O)(=O)Nc1cc(Sc2nc3ccccc3s2)c(O)c2ccccc12] | C27H19N3O5S3 |
| 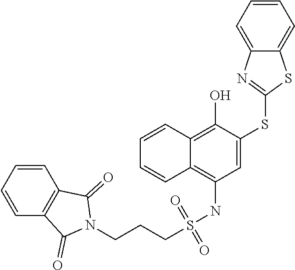 [SMILES: O=C1c2ccccc2C(=O)N1CCCS(=O)(=O)Nc1cc(Sc2nc3ccccc3s2)c(O)c2ccccc12] | C28H21N3O5S3 |
| 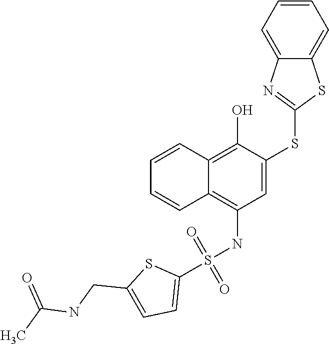 [SMILES: CC(=O)NCc1ccc(S(=O)(=O)Nc2cc(Sc3nc4ccccc4s3)c(O)c3ccccc23)s1] | C24H19N3O4S4 |
| 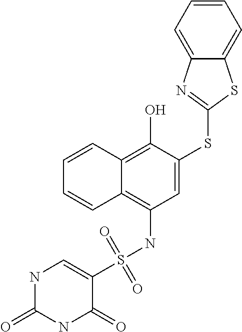 [SMILES: O=c1[nH]cc(S(=O)(=O)Nc2cc(Sc3nc4ccccc4s3)c(O)c3ccccc23)c(=O)[nH]1] | C21H14N4O5S3 |
| 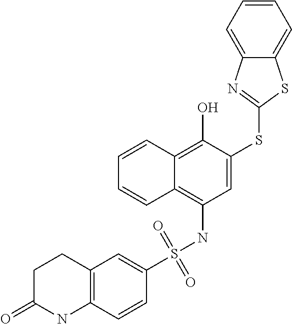 [SMILES: O=C1CCc2cc(S(=O)(=O)Nc3cc(Sc4nc5ccccc5s4)c(O)c4ccccc34)ccc2N1] | C26H19N3O4S3 |
| 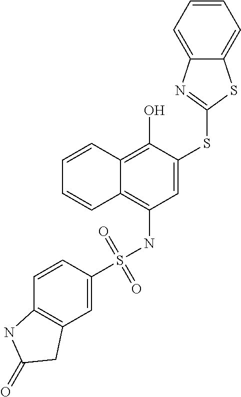 [SMILES: O=C1Cc2cc(S(=O)(=O)Nc3cc(Sc4nc5ccccc5s4)c(O)c4ccccc34)ccc2N1] | C25H17N3O4S3 |
| 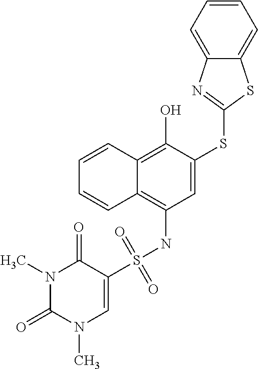 [SMILES: Cn1cc(S(=O)(=O)Nc2cc(Sc3nc4ccccc4s3)c(O)c3ccccc23)c(=O)n(C)c1=O] | C23H18N4O5S3 |
| 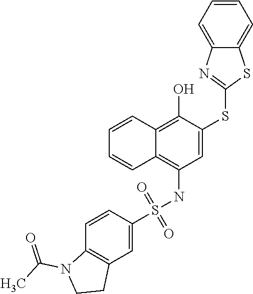 [SMILES: CC(=O)N1CCc2cc(S(=O)(=O)Nc3cc(Sc4nc5ccccc5s4)c(O)c4ccccc34)ccc21] | C27H21N3O4S3 |
| 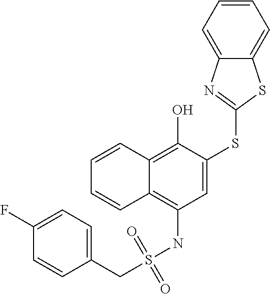 [SMILES: O=S(=O)(Cc1ccc(F)cc1)Nc1cc(Sc2nc3ccccc3s2)c(O)c2ccccc12] | C24H17FN2O3S3 |
| 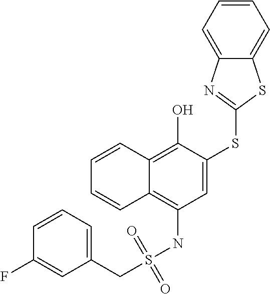 [SMILES: O=S(=O)(Cc1cccc(F)c1)Nc1cc(Sc2nc3ccccc3s2)c(O)c2ccccc12] | C24H17FN2O3S3 |
| 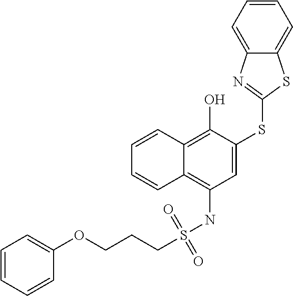 [SMILES: O=S(=O)(CCCOc1ccccc1)Nc1cc(Sc2nc3ccccc3s2)c(O)c2ccccc12] | C26H22N2O4S3 |
| 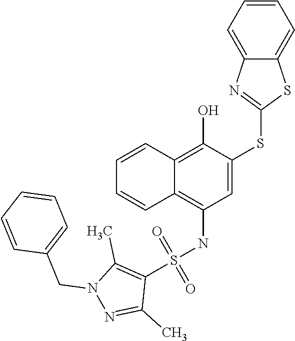 [SMILES: Cc1nn(Cc2ccccc2)c(C)c1S(=O)(=O)Nc1cc(Sc2nc3ccccc3s2)c(O)c2ccccc12] | C29H24N4O3S3 |
| 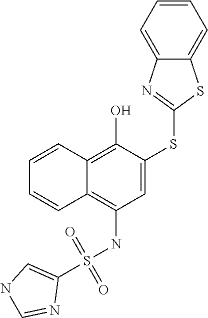 [SMILES: O=S(=O)(Nc1cc(Sc2nc3ccccc3s2)c(O)c2ccccc12)c1c[nH]cn1] | C20H14N4O3S3 |
| 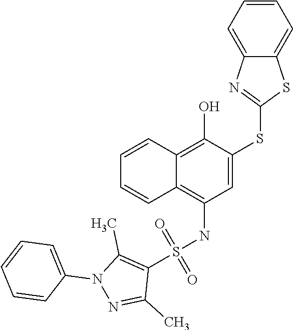 [SMILES: Cc1nn(-c2ccccc2)c(C)c1S(=O)(=O)Nc1cc(Sc2nc3ccccc3s2)c(O)c2ccccc12] | C28H22N4O3S3 |
| 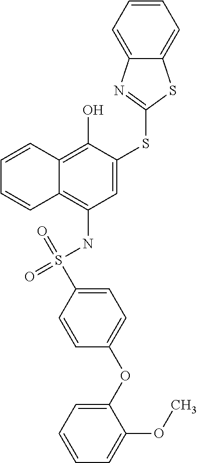 [SMILES: COc1ccccc1Oc1ccc(S(=O)(=O)Nc2cc(Sc3nc4ccccc4s3)c(O)c3ccccc23)cc1] | C30H22N2O5S3 |
| 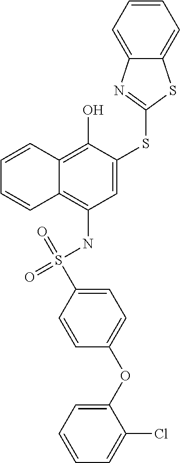 [SMILES: O=S(=O)(Nc1cc(Sc2nc3ccccc3s2)c(O)c2ccccc12)c1ccc(Oc2ccccc2Cl)cc1] | C29H19ClN2O4S3 |
| 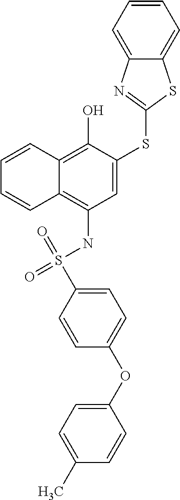 [SMILES: Cc1ccc(Oc2ccc(S(=O)(=O)Nc3cc(Sc4nc5ccccc5s4)c(O)c4ccccc34)cc2)cc1] | C30H22N2O4S3 |
| 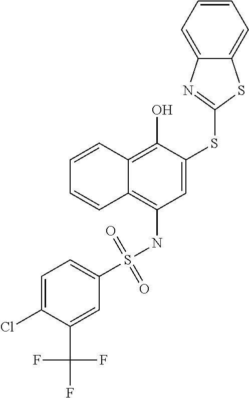 [SMILES: O=S(=O)(Nc1cc(Sc2nc3ccccc3s2)c(O)c2ccccc12)c1ccc(Cl)c(C(F)(F)F)c1] | C24H14ClF3N2O3S3 |
| 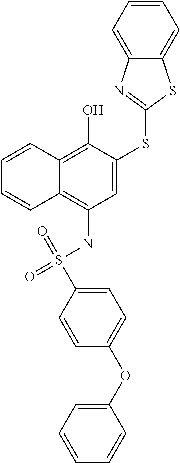 [SMILES: O=S(=O)(Nc1cc(Sc2nc3ccccc3s2)c(O)c2ccccc12)c1ccc(Oc2ccccc2)cc1] | C29H20N2O4S3 |
| 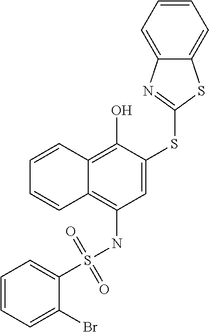 [SMILES: O=S(=O)(Nc1cc(Sc2nc3ccccc3s2)c(O)c2ccccc12)c1ccccc1Br] | C23H15BrN2O3S3 |
| 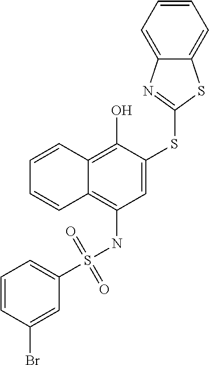 [SMILES: O=S(=O)(Nc1cc(Sc2nc3ccccc3s2)c(O)c2ccccc12)c1cccc(Br)c1] | C23H15BrN2O3S3 |
| 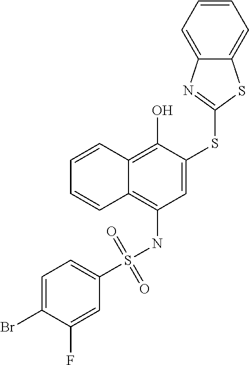 [SMILES: O=S(=O)(Nc1cc(Sc2nc3ccccc3s2)c(O)c2ccccc12)c1ccc(Br)c(F)c1] | C23H14BrFN2O3S3 |
| 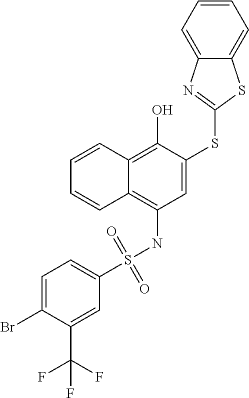 [SMILES: O=S(=O)(Nc1cc(Sc2nc3ccccc3s2)c(O)c2ccccc12)c1ccc(Br)c(C(F)(F)F)c1] | C24H14BrF3N2O3S3 |
| 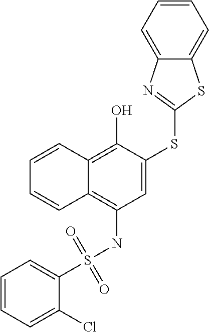 [SMILES: O=S(=O)(Nc1cc(Sc2nc3ccccc3s2)c(O)c2ccccc12)c1ccccc1Cl] | C23H15ClN2O3S3 |
| 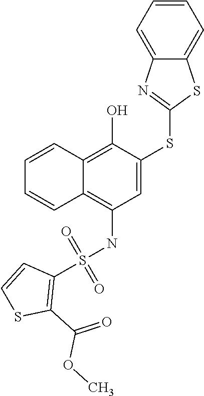 [SMILES: COC(=O)c1sccc1S(=O)(=O)Nc1cc(Sc2nc3ccccc3s2)c(O)c2ccccc12] | C23H16N2O5S4 |
| 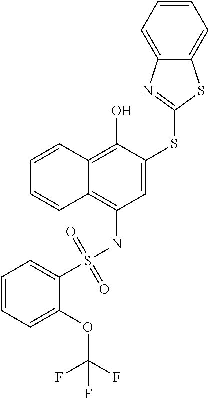 [SMILES: O=S(=O)(Nc1cc(Sc2nc3ccccc3s2)c(O)c2ccccc12)c1ccccc1OC(F)(F)F] | C24H15F3N2O4S3 |

TABLE 7

| Structure | Formula structure |
|---|---|
| 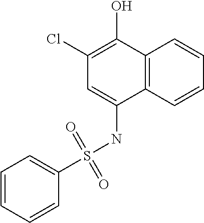 [SMILES: O=S(=O)(Nc1cc(Cl)c(O)c2ccccc12)c1ccccc1] | C16H12ClNO3S |
| 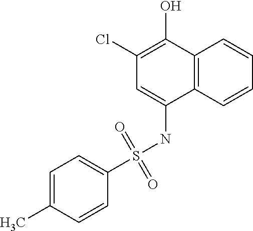 [SMILES: Cc1ccc(S(=O)(=O)Nc2cc(Cl)c(O)c3ccccc23)cc1] | C17H14ClNO3S |
| 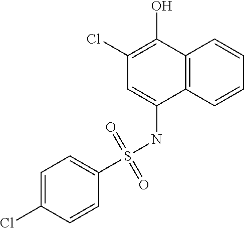 [SMILES: O=S(=O)(Nc1cc(Cl)c(O)c2ccccc12)c1ccc(Cl)cc1] | C16H11Cl2NO3S |
| 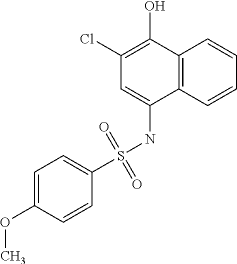 [SMILES: COc1ccc(S(=O)(=O)Nc2cc(Cl)c(O)c3ccccc23)cc1] | C17H14ClNO4S |
| 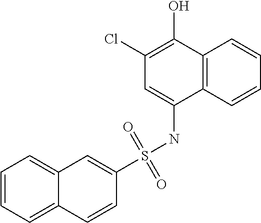 [SMILES: O=S(=O)(Nc1cc(Cl)c(O)c2ccccc12)c1ccc2ccccc2c1] | C20H14ClNO3S |
| 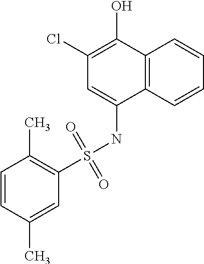 [SMILES: Cc1ccc(C)c(S(=O)(=O)Nc2cc(Cl)c(O)c3ccccc23)c1] | C18H16ClNO3S |
| 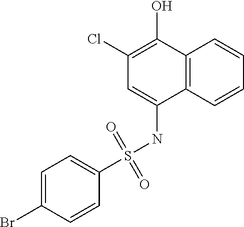 [SMILES: O=S(=O)(Nc1cc(Cl)c(O)c2ccccc12)c1ccc(Br)cc1] | C16H11BrClNO3S |
| 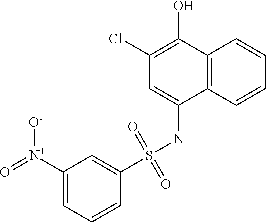 [SMILES: O=[N+]([O-])c1cccc(S(=O)(=O)Nc2cc(Cl)c(O)c3ccccc23)c1] | C16H11ClN2O5S |
| 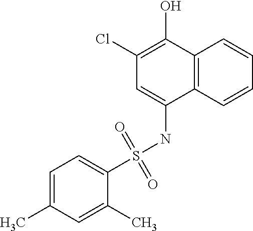 [SMILES: Cc1ccc(S(=O)(=O)Nc2cc(Cl)c(O)c3ccccc23)c(C)c1] | C18H16ClNO3S |
| 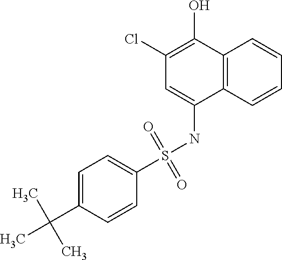 [SMILES: CC(C)(C)c1ccc(S(=O)(=O)Nc2cc(Cl)c(O)c3ccccc23)cc1] | C20H20ClNO3S |
| 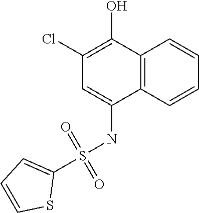 [SMILES: O=S(=O)(Nc1cc(Cl)c(O)c2ccccc12)c1cccs1] | C14H10ClNO3S2 |
| 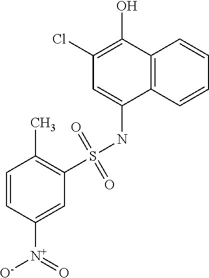 [SMILES: Cc1ccc([N+](=O)[O-])cc1S(=O)(=O)Nc1cc(Cl)c(O)c2ccccc12] | C17H13ClN2O5S |
| 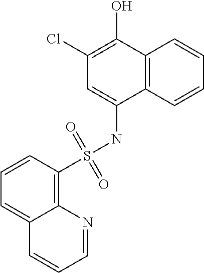 [SMILES: O=S(=O)(Nc1cc(Cl)c(O)c2ccccc12)c1cccc2cccnc12] | C19H13ClN2O3S |
| 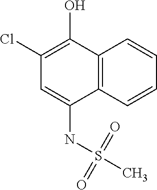 [SMILES: CS(=O)(=O)Nc1cc(Cl)c(O)c2ccccc12] | C11H10ClNO3S |
| 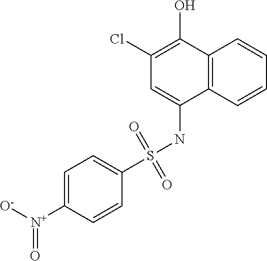 [SMILES: O=[N+]([O-])c1ccc(S(=O)(=O)Nc2cc(Cl)c(O)c3ccccc23)cc1] | C16H11ClN2O5S |
| 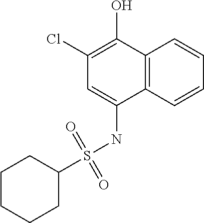 [SMILES: O=S(=O)(Nc1cc(Cl)c(O)c2ccccc12)C1CCCCC1] | C16H18ClNO3S |
| 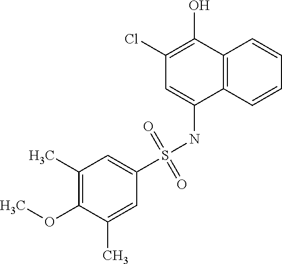 [SMILES: COc1c(C)cc(S(=O)(=O)Nc2cc(Cl)c(O)c3ccccc23)cc1C] | C19H18ClNO4S |
| 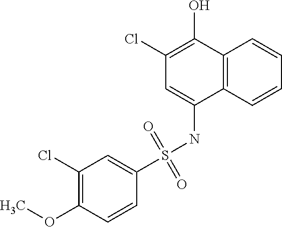 [SMILES: COc1ccc(S(=O)(=O)Nc2cc(Cl)c(O)c3ccccc23)cc1Cl] | C17H13Cl2NO4S |
| 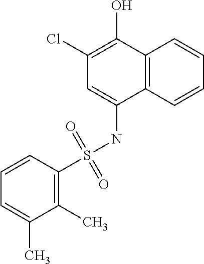 [SMILES: Cc1cccc(S(=O)(=O)Nc2cc(Cl)c(O)c3ccccc23)c1C] | C18H16ClNO3S |
| 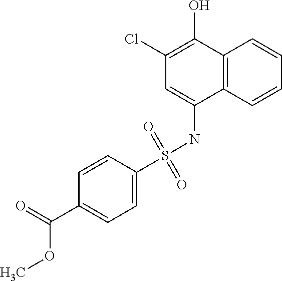 [SMILES: COC(=O)c1ccc(S(=O)(=O)Nc2cc(Cl)c(O)c3ccccc23)cc1] | C18H14ClNO5S |
| 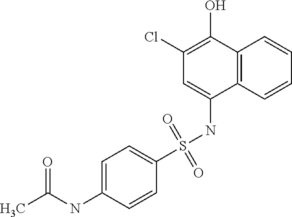 [SMILES: CC(=O)Nc1ccc(S(=O)(=O)Nc2cc(Cl)c(O)c3ccccc23)cc1] | C18H15ClN2O4S |
| 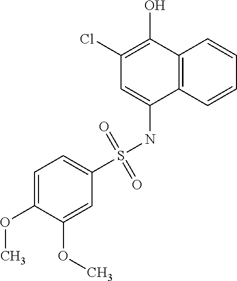 [SMILES: COc1ccc(S(=O)(=O)Nc2cc(Cl)c(O)c3ccccc23)cc1OC] | C18H16ClNO5S |
| 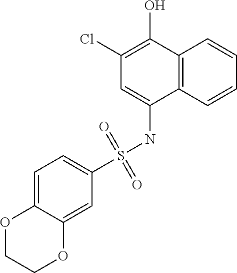 [SMILES: O=S(=O)(Nc1cc(Cl)c(O)c2ccccc12)c1ccc2c(c1)OCCO2] | C18H14ClNO5S |
| 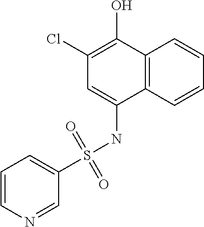 [SMILES: O=S(=O)(Nc1cc(Cl)c(O)c2ccccc12)c1cccnc1] | C15H11ClN2O3S |
| 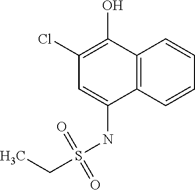 [SMILES: CCS(=O)(=O)Nc1cc(Cl)c(O)c2ccccc12] | C12H12ClNO3S |
| 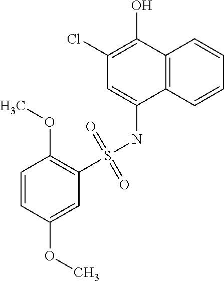 [SMILES: COc1ccc(OC)c(S(=O)(=O)Nc2cc(Cl)c(O)c3ccccc23)c1] | C18H16ClNO5S |
| 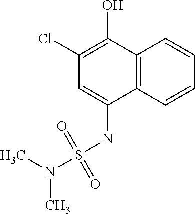 [SMILES: CN(C)S(=O)(=O)Nc1cc(Cl)c(O)c2ccccc12] | C12H13ClN2O3S |
| 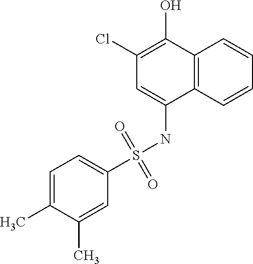 [SMILES: Cc1ccc(S(=O)(=O)Nc2cc(Cl)c(O)c3ccccc23)cc1C] | C18H16ClNO3S |
| 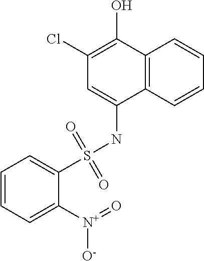 [SMILES: O=[N+]([O-])c1ccccc1S(=O)(=O)Nc1cc(Cl)c(O)c2ccccc12] | C16H11ClN2O5S |
| 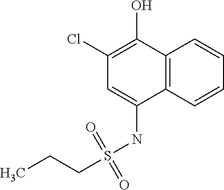 [SMILES: CCCS(=O)(=O)Nc1cc(Cl)c(O)c2ccccc12] | C13H14ClNO3S |
| 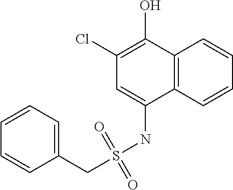 [SMILES: O=S(=O)(Cc1ccccc1)Nc1cc(Cl)c(O)c2ccccc12] | C17H14ClNO3S |
| 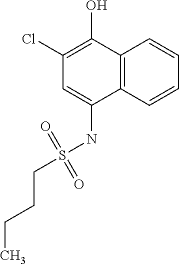 [SMILES: CCCCS(=O)(=O)Nc1cc(Cl)c(O)c2ccccc12] | C14H16ClNO3S |
| 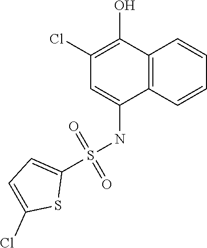 [SMILES: O=S(=O)(Nc1cc(Cl)c(O)c2ccccc12)c1ccc(Cl)s1] | C14H9Cl2NO3S2 |
| 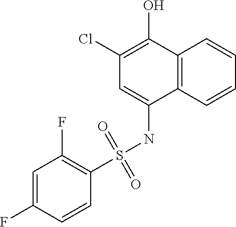 [SMILES: O=S(=O)(Nc1cc(Cl)c(O)c2ccccc12)c1ccc(F)cc1F] | C16H10ClF2NO3S |
| 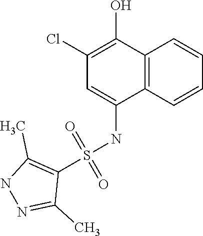 [SMILES: Cc1n[nH]c(C)c1S(=O)(=O)Nc1cc(Cl)c(O)c2ccccc12] | C15H14ClN3O3S |
| 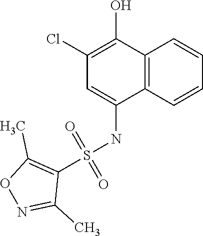 [SMILES: Cc1noc(C)c1S(=O)(=O)Nc1cc(Cl)c(O)c2ccccc12] | C15H13ClN2O4S |
| 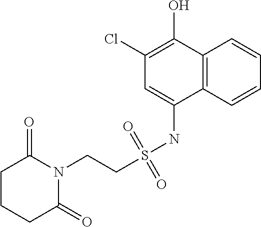 [SMILES: O=C1CCCC(=O)N1CCS(=O)(=O)Nc1cc(Cl)c(O)c2ccccc12] | C17H17ClN2O5S |
| 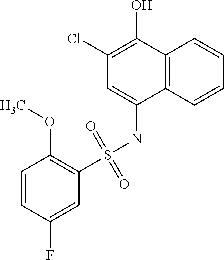 [SMILES: COc1ccc(F)cc1S(=O)(=O)Nc1cc(Cl)c(O)c2ccccc12] | C17H13ClFNO4S |
| 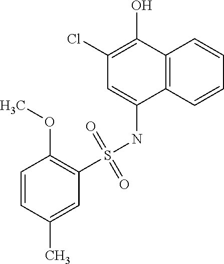 [SMILES: COc1ccc(C)cc1S(=O)(=O)Nc1cc(Cl)c(O)c2ccccc12] | C18H16ClNO4S |
| 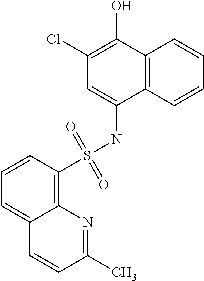 [SMILES: Cc1ccc2cccc(S(=O)(=O)Nc3cc(Cl)c(O)c4ccccc34)c2n1] | C20H15ClN2O3S |
| 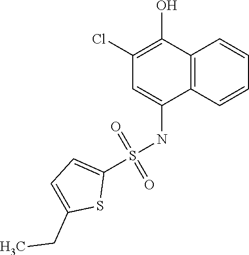 [SMILES: CCc1ccc(S(=O)(=O)Nc2cc(Cl)c(O)c3ccccc23)s1] | C16H14ClNO3S2 |
| 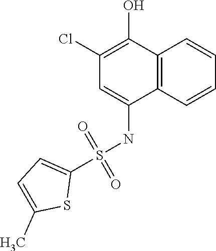 [SMILES: Cc1ccc(S(=O)(=O)Nc2cc(Cl)c(O)c3ccccc23)s1] | C15H12ClNO3S2 |
| 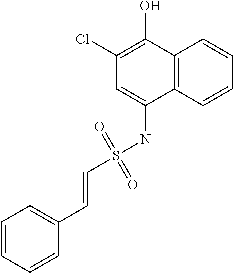 [SMILES: O=S(=O)(/C=C/c1ccccc1)Nc1cc(Cl)c(O)c2ccccc12] | C18H14ClNO3S |
| 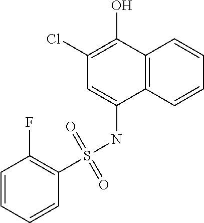 [SMILES: O=S(=O)(Nc1cc(Cl)c(O)c2ccccc12)c1ccccc1F] | C16H11ClFNO3S |
| 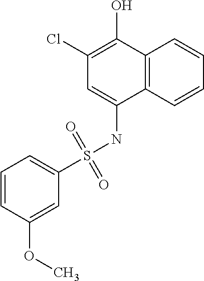 [SMILES: COc1cccc(S(=O)(=O)Nc2cc(Cl)c(O)c3ccccc23)c1] | C17H14ClNO4S |
| 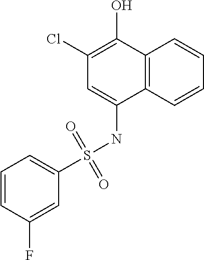 [SMILES: O=S(=O)(Nc1cc(Cl)c(O)c2ccccc12)c1cccc(F)c1] | C16H11ClFNO3S |
| 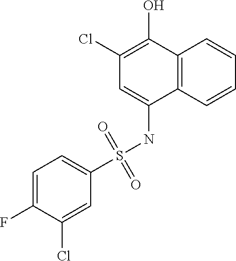 [SMILES: O=S(=O)(Nc1cc(Cl)c(O)c2ccccc12)c1ccc(F)c(Cl)c1] | C16H10Cl2FNO3S |
| 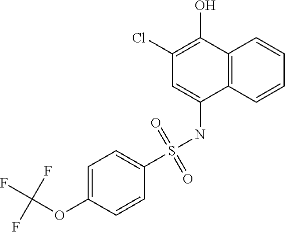 [SMILES: O=S(=O)(Nc1cc(Cl)c(O)c2ccccc12)c1ccc(OC(F)(F)F)cc1] | C17H11ClF3NO4S |
| 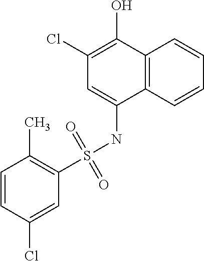 [SMILES: Cc1ccc(Cl)cc1S(=O)(=O)Nc1cc(Cl)c(O)c2ccccc12] | C17H13Cl2NO3S |
| 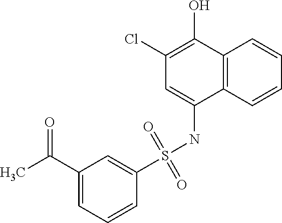 [SMILES: CC(=O)c1cccc(S(=O)(=O)Nc2cc(Cl)c(O)c3ccccc23)c1] | C18H14ClNO4S |
| 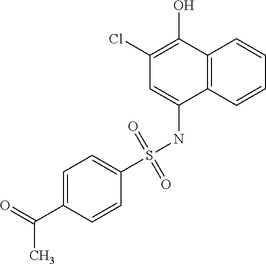 [SMILES: CC(=O)c1ccc(S(=O)(=O)Nc2cc(Cl)c(O)c3ccccc23)cc1] | C18H14ClNO4S |
| 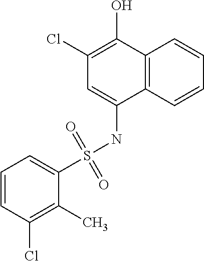 [SMILES: Cc1c(Cl)cccc1S(=O)(=O)Nc1cc(Cl)c(O)c2ccccc12] | C17H13Cl2NO3S |
| 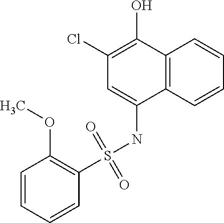 [SMILES: COc1ccccc1S(=O)(=O)Nc1cc(Cl)c(O)c2ccccc12] | C17H14ClNO4S |
| 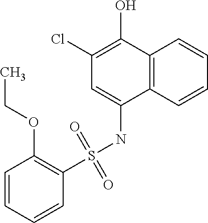 [SMILES: CCOc1ccccc1S(=O)(=O)Nc1cc(Cl)c(O)c2ccccc12] | C18H16ClNO4S |
| 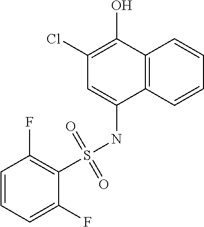 [SMILES: O=S(=O)(Nc1cc(Cl)c(O)c2ccccc12)c1c(F)cccc1F] | C16H10ClF2NO3S |
| 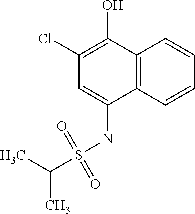 [SMILES: CC(C)S(=O)(=O)Nc1cc(Cl)c(O)c2ccccc12] | C13H14ClNO3S |
| 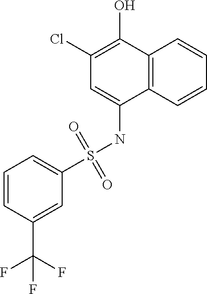 [SMILES: O=S(=O)(Nc1cc(Cl)c(O)c2ccccc12)c1cccc(C(F)(F)F)c1] | C17H11ClF3NO3S |
| 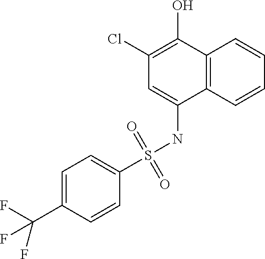 [SMILES: O=S(=O)(Nc1cc(Cl)c(O)c2ccccc12)c1ccc(C(F)(F)F)cc1] | C17H11ClF3NO3S |
| 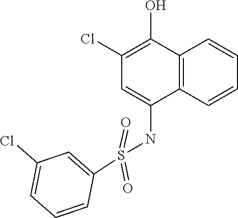 [SMILES: O=S(=O)(Nc1cc(Cl)c(O)c2ccccc12)c1cccc(Cl)c1] | C16H11Cl2NO3S |
| 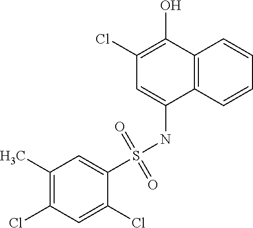 [SMILES: Cc1cc(S(=O)(=O)Nc2cc(Cl)c(O)c3ccccc23)c(Cl)cc1Cl] | C17H12Cl3NO3S |
| 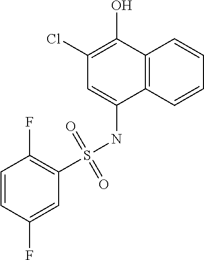 [SMILES: O=S(=O)(Nc1cc(Cl)c(O)c2ccccc12)c1cc(F)ccc1F] | C16H10ClF2NO3S |
| 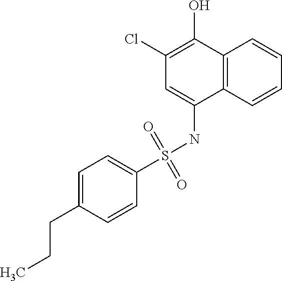 [SMILES: CCCc1ccc(S(=O)(=O)Nc2cc(Cl)c(O)c3ccccc23)cc1] | C19H18ClNO3S |
| 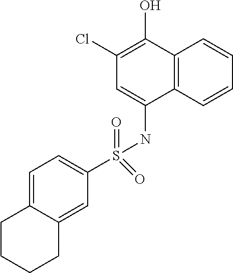 [SMILES: O=S(=O)(Nc1cc(Cl)c(O)c2ccccc12)c1ccc2c(c1)CCCC2] | C20H18ClNO3S |
| 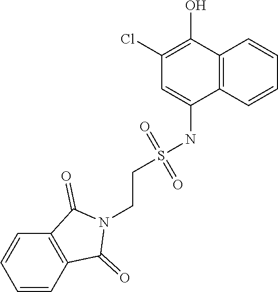 [SMILES: O=C1c2ccccc2C(=O)N1CCS(=O)(=O)Nc1cc(Cl)c(O)c2ccccc12] | C20H15ClN2O5S |
| 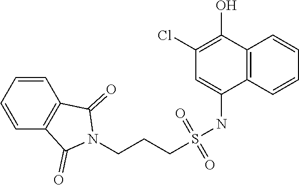 [SMILES: O=C1c2ccccc2C(=O)N1CCCS(=O)(=O)Nc1cc(Cl)c(O)c2ccccc12] | C21H17ClN2O5S |
| 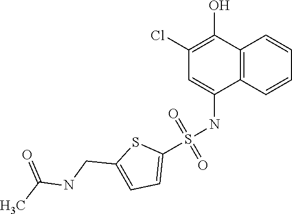 [SMILES: CC(=O)NCc1ccc(S(=O)(=O)Nc2cc(Cl)c(O)c3ccccc23)s1] | C17H15ClN2O4S2 |
| 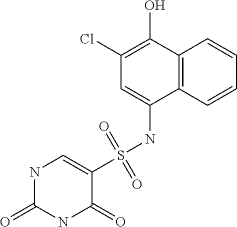 [SMILES: O=c1[nH]cc(S(=O)(=O)Nc2cc(Cl)c(O)c3ccccc23)c(=O)[nH]1] | C14H10ClN3O5S |
| 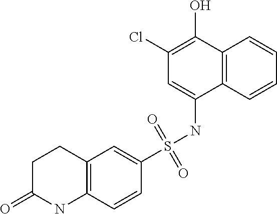 [SMILES: O=C1CCc2cc(S(=O)(=O)Nc3cc(Cl)c(O)c4ccccc34)ccc2N1] | C19H15ClN2O4S |
| 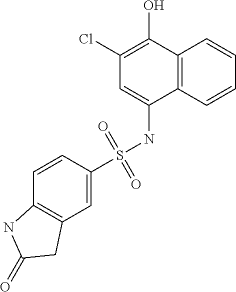 [SMILES: O=C1Cc2cc(S(=O)(=O)Nc3cc(Cl)c(O)c4ccccc34)ccc2N1] | C18H13ClN2O4S |
| 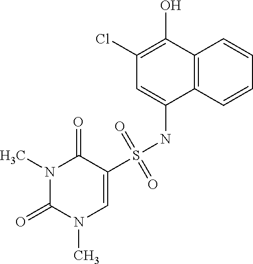 [SMILES: Cn1cc(S(=O)(=O)Nc2cc(Cl)c(O)c3ccccc23)c(=O)n(C)c1=O] | C16H14ClN3O5S |
| 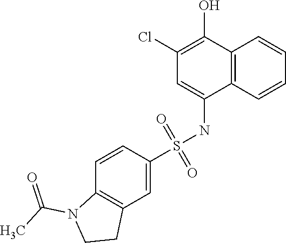 [SMILES: CC(=O)N1CCc2cc(S(=O)(=O)Nc3cc(Cl)c(O)c4ccccc34)ccc21] | C20H17ClN2O4S |
| 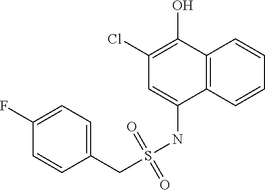 [SMILES: O=S(=O)(Cc1ccc(F)cc1)Nc1cc(Cl)c(O)c2ccccc12] | C17H13ClFNO3S |
| 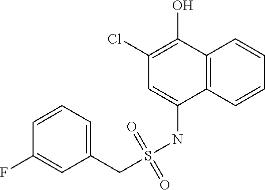 [SMILES: O=S(=O)(Cc1cccc(F)c1)Nc1cc(Cl)c(O)c2ccccc12] | C17H13ClFNO3S |
| 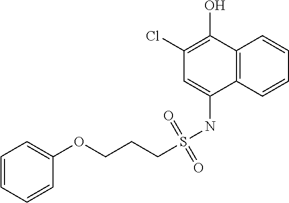 [SMILES: O=S(=O)(CCCOc1ccccc1)Nc1cc(Cl)c(O)c2ccccc12] | C19H18ClNO4S |
| 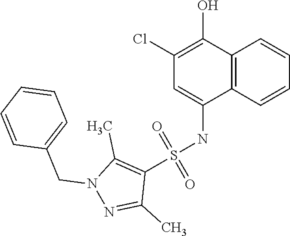 [SMILES: Cc1nn(Cc2ccccc2)c(C)c1S(=O)(=O)Nc1cc(Cl)c(O)c2ccccc12] | C22H20ClN3O3S |
| 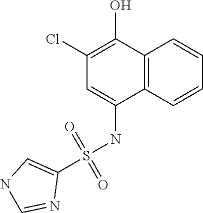 [SMILES: O=S(=O)(Nc1cc(Cl)c(O)c2ccccc12)c1c[nH]cn1] | C13H10ClN3O3S |
| 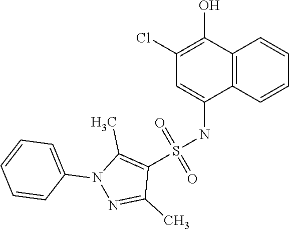 [SMILES: Cc1nn(-c2ccccc2)c(C)c1S(=O)(=O)Nc1cc(Cl)c(O)c2ccccc12] | C21H18ClN3O3S |
| 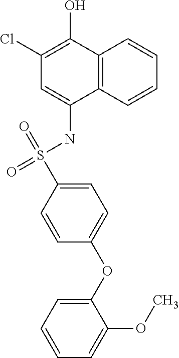 [SMILES: COc1ccccc1Oc1ccc(S(=O)(=O)Nc2cc(Cl)c(O)c3ccccc23)cc1] | C23H18ClNO5S |
| 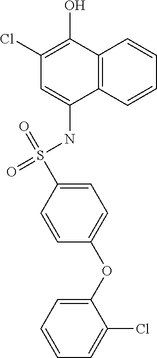 [SMILES: O=S(=O)(Nc1cc(Cl)c(O)c2ccccc12)c1ccc(Oc2ccccc2Cl)cc1] | C22H15Cl2NO4S |
| 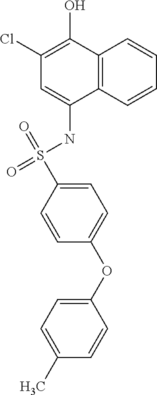 [SMILES: Cc1ccc(Oc2ccc(S(=O)(=O)Nc3cc(Cl)c(O)c4ccccc34)cc2)cc1] | C23H18ClNO4S |
| 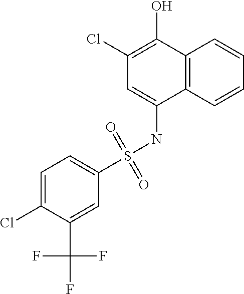 [SMILES: O=S(=O)(Nc1cc(Cl)c(O)c2ccccc12)c1ccc(Cl)c(C(F)(F)F)c1] | C17H10Cl2F3NO3S |
| 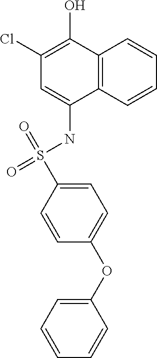 [SMILES: O=S(=O)(Nc1cc(Cl)c(O)c2ccccc12)c1ccc(Oc2ccccc2)cc1] | C22H16ClNO4S |
| 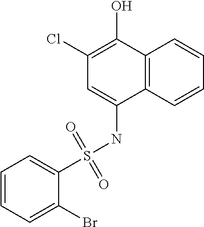 [SMILES: O=S(=O)(Nc1cc(Cl)c(O)c2ccccc12)c1ccccc1Br] | C16H11BrClNO3S |
| 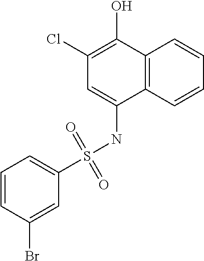 [SMILES: O=S(=O)(Nc1cc(Cl)c(O)c2ccccc12)c1cccc(Br)c1] | C16H11BrClNO3S |
| 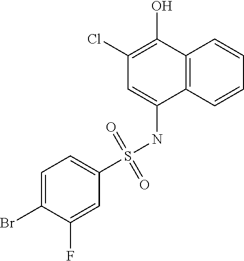 [SMILES: O=S(=O)(Nc1cc(Cl)c(O)c2ccccc12)c1ccc(Br)c(F)c1] | C16H10BrClFNO3S |
| 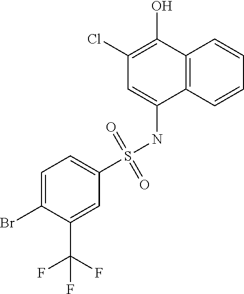 [SMILES: O=S(=O)(Nc1cc(Cl)c(O)c2ccccc12)c1ccc(Br)c(C(F)(F)F)c1] | C17H10BrClF3NO3S |
| 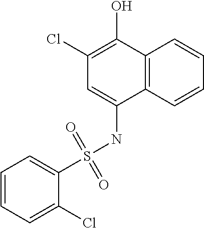 [SMILES: O=S(=O)(Nc1cc(Cl)c(O)c2ccccc12)c1ccccc1Cl] | C16H11Cl2NO3S |
| 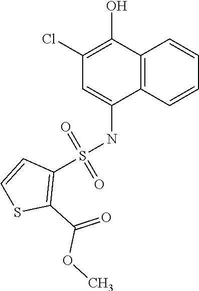 [SMILES: COC(=O)c1sccc1S(=O)(=O)Nc1cc(Cl)c(O)c2ccccc12] | C16H12ClNO5S2 |
| 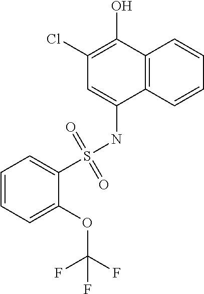 [SMILES: O=S(=O)(Nc1cc(Cl)c(O)c2ccccc12)c1ccccc1OC(F)(F)F] | C17H11ClF3NO4S |

Any compound disclosed herein for use with any method disclosed herein is delivered occur by any suitable route, including systemic or local, although in specific embodiments, the delivery route is oral, intravenous, topical, subcutaneous, intraarterial, intraperitoneal, buccal, by aerosol, by inhalation, and so forth, for example.

Individuals subjected to methods of the disclosure may be exposed to one or more doses of STAT3 inhibitors, and each dose may have one or more STAT3 inhibitors. Multiple doses may span any suitable duration there between, such as 1-24 hours, 1-7 days, 1-4 weeks, or 1-12 months between doses. Multiple doses may be daily, weekly, biweekly, monthly, yearly, and so forth. An individual may be administered one STAT3 inhibitor at a particular dose and a different STAT3 inhibitor at a subsequent dose.

In particular embodiments, a suitable dose for any Stat3 inhibitor for methods of treatment or prevention is about 25-50 mg/kg or 1.75-3.5 grams for an individual weighing 70 kg. In other embodiments, for insulin resistance the dosage for a Stat3 inhibitor is less than 25-50 mg/kg or 1.75-3.5 grams for an individual weighing 70 kg.

### Combination Therapy

In some embodiments of a method disclosed herein, the method further comprising administering an additional agent or therapy method such as another insulin resistance treatment or prevention and/or a treatment for an underlying condition associated with insulin resistance. The compounds (which may or may not be a STAT3 inhibitor) may precede or follow the other agent treatment by intervals ranging from minutes to weeks, for example. In embodiments where the other agent and the compounds of the disclosure are applied separately to an individual with insulin resistance, such as upon delivery to an individual suspected of having insulin resistance, known to have insulin resistance, or at risk for having insulin resistance, one would generally ensure that a significant period of time did not expire between the time of each delivery, such that the agent and compounds of the disclosure would still be able to exert an advantageously combined effect on the individual.

In particular embodiments, the individual in addition to being subjected to STAT3 inhibitor methods of the disclosure will be subjected to one or more other therapies that treat or reverse insulin resistance and/or any associated medical condition. Examples include exercise, cessation of smoking, reduction of sugar intake, healthy diet, intake of omega-3 fatty acids, stress reduction, or a combination thereof.

In specific embodiments, it is contemplated that one may contact the cell, tissue or individual with one, two, three, four or more modalities substantially simultaneously (i.e., within less than about a minute) with the compounds of the disclosure. In other aspects, one or more agents may be administered within about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, to about 48 hours or more prior to and/or after administering the compounds of the disclosure. In certain other embodiments, an agent may be administered within of from about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20, to about 21 days prior to and/or after administering the compounds of the disclosure, for example. In some situations, it may be desirable to extend the time period for treatment significantly, such as where several weeks (e.g., about 1, about 2, about 3, about 4, about 5, about 6, about 7 or about 8 weeks or more) lapse between the respective administrations. In some situations, it may be desirable to extend the time period for treatment significantly, such as where several months (e.g., about 1, about 2, about 3, about 4, about 5, about 6, about 7 or about 8 weeks or more) lapse between the respective administrations.

Administration of the therapeutic compounds of the present disclosure to an individual will follow general protocols for the administration of drugs, taking into account the toxicity. It is expected that the treatment cycles would be repeated as necessary.

### Pharmaceutical Preparations of STAT3 Inhibitors

Pharmaceutical compositions for use with the methods disclosed herein comprise an effective amount of one or more STAT3 inhibitors disclosed herein dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that comprises at least one STAT3 inhibitor will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The compositions comprising the STAT3 inhibitors disclosed herein may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The compositions comprising the STAT3 inhibitor may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the compositions of the present disclosure suitable for administration are provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present disclosure is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include one or more STAT3 inhibitors and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the one or more STAT3 inhibitors may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Alimentary Compositions and Formulations

In preferred embodiments of the present disclosure, the one or more STATS inhibitors are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the STAT3 inhibitor compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### Parenteral Compositions and Formulations

In further embodiments, one or more STAT3 inhibitors may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### Miscellaneous Pharmaceutical Compositions and Formulations

In other particular embodiments of the disclosure, the STAT3 inhibitor may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### Kits of the Disclosure

Any of the compounds or compositions described herein may be comprised in a kit. In a non-limiting example, one or more Stat3 inhibitors are comprised in a kit. The Stat3 inhibitor components of the kits may be packaged either in aqueous media or in lyophilized form. The container means of the kits will generally include at least one vial, test tube, flask, bottle, syringe or other container means, into which a Stat3 inhibitor may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the Stat3 inhibitor and any other reagent containers in close confinement for commercial sale. Such containers may include injection or blow-molded plastic containers into which the desired vials are retained.

The Stat3 inhibitors of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the disclosure may also comprise, and/or be packaged with, an instrument for assisting with the injection/administration and/or placement of the ultimate composition within the body of an animal. Such an instrument may be a syringe, pipette, forceps, and/or any such medically approved delivery vehicle.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Mechanism Underlying the Development of Insulin Resistance in Models of CKD or High Fat Diet

Methods

Animals: The experimental procedures were approved by the Institutional Animal Care and Use Committee of Baylor College of Medicine. Wild type (WT) mice (C57BL/6) were purchased from Jackson lab (Bar Harbor, Me.). To create a model of CKD, 8-10 week old mice underwent subtotal nephrectomy or sham-operated control as described; ³¹ CKD mice with a BUN ˜80 mg/dl were studied. CKD or sham-operated control mice were assigned to two subgroups: one subgroup was injected intraperitoneally with TTI-101 (12.5 mg/kg body weight in D5W) every other day for 2 weeks, while the other subgroup received an identical volume of D5W for 2 weeks.

Another model was generated of insulin resistance by feeding wild type mice a high fat diet (HFD: 58% kcal from fat, Research Diets, New Brunswick, N.J.) for 12 weeks while control mice were fed the regular diet (RD: 11% kcal from fat). To study the effect of p-Stat3 on insulin resistance, HFD-fed mice were randomly assigned to two subgroups: one subgroup was injected intraperitoneally (i.p.) with TTI-101 (12.5 mg/kg body weight in D5W) every other day for 4 weeks, while the other subgroup received an identical volume of D5W for 4 weeks.

Stat3 KO mice were created by crossing mice expressing Floxed-Stat3 with mice expressing muscle creatine kinase Cre (MCK-Cre) as described.³³ Beginning at four weeks after birth, Floxed-Stat3 or Stat3 KO mice were fed the HFD for 16 weeks.

For glucose tolerance tests (GTT), mice with free access to water were fasted for 16 hrs and then injected intraperitoneally (i.p.) with 2 mg/kg glucose and tail vein blood was collected at 0, 30, 60 and 120 min intervals to measure blood glucose concentrations using a True Track Glucometer. For insulin tolerance test (ITT), mice were fasted for 4 hrs, then were injected i.p. with 2 units/kg insulin; tail vein blood was collected after 0, 30, 60 and 120 min to measure blood glucose concentrations. Changes in blood glucose were analyzed as the “area under curve” (AUC) method using Statstodo program (http://www.statstodo.com/AUC_Exp.php).

Cell culture: Mouse C2C12 myoblasts were obtained from American Type Culture Collection (ATCC, Manassas, Va.). Cells were transfected with Fbxo40 SiRNA (Santa Cruze Biotechnology, Dallas, Tex.) or its control SiRNA using the Invitrogen Neon transfection system (Invitrogen Madison, Wis.). To induce differentiation, C2C12 myoblasts were grown to 85% confluence and then switched to differentiation media consisting of DMEM plus 2% HS and 1% P/S (PS; Invitrogen Madison, Wis.). The myotubes were treated with/without 100 ng/ml IL-6 (Biolegend, San Diego, Calif.) for 24 h. Cell lysates were subjected to western blotting.

Luciferase reporter assays: The human Fbxo40 promoter was cloned into a Gaussia-luciferase reporter that was obtained from GeneCopoeia, Inc. (Rockville, Md.). The 1226 bp Fbxo40 promoter sequences included 1062 bp upstream and 163 bp downstream. The potential Stat3 binding site, TTCCAGGAA, is located upstream from 520 to 529 bp. The Fbxo40 promoter clone and plasmid expressing constitutively active Stat3 or cDNA3 were transfected into C2C12 myoblasts using the Invitrogen Neon transfection system. At 24 h after transfection, the activity of Gaussia luciferase was measured using the Thermo Scientific™ Pierce™ Gaussia Luciferase Flash Assay Kit.

Chromatin Immunoprecipitation (CHIP) Assays: C2C12 myoblasts were transfected with plasmids expressing Stat3C or GFP using the Invitrogen Neon transfection system. C2C12 cells were differentiated for 24 h before treatment with 1% formaldehyde (Sigma-Aldrich) for 10 min. Cells were washed 3× with ice-cold PBS containing a protease inhibitor (Sigma-Aldrich, St. Louis). Myotubes were then lysed, vortexed and sonicated according to Millipore Kit manufacturer's instructions as described.³⁴ After centrifugation, the protein-DNA lysate was diluted 10-fold in CHIP buffer and precleared using salmon sperm DNA and protein A/G agarose beads for 1 h at 4° C. Each 100 μL of the protein-DNA lysate was used as an input control. Cellular protein-DNA lysates were immunoprecipitated overnight at 4° C. with antibodies against Stat3 or rabbit IgG (Santa Cruz Biotechnology, Dallas, Tex.). Subsequently, lysates were incubated with protein A/G Agarose beads (SCBT) for 1 h at 4° C. The complexes were washed as described by the manufacturer. Immunoprecipitated DNA was then reverse cross linked at 65° C. for 4 h in the presence of 0.2 M NaCl; the mixture was purified using phenol/chloroform/isoamyl alcohol. A total of 5 μl of the purified DNA was subjected to PCR amplification using primers that cover the Stat3 binding sites in the mouse Fbxo40 promoter. The primers were purchased from Sabiosciences (Frederick, Md.). The fold enrichment of Stat3 relative to IgG was calculated.

RNA extraction and quantitative real-time PCR (qPCR): RNAs were isolated using the RNeasy kit (Qiagen, Valencia, Calif.) as instructed by the company. RT-PCR was performed to obtain relative gene expressions by calculating cycle threshold (Ct) values using GAPDH as an internal control (relative expression=2(sample Ct−GAPDH Ct)).³⁵ Sequences of primers will be provided upon request.

Antibodies: The primary antibodies of p-Akt (Ser473) (D9E) #4060, Akt (40D4)#2920, p-Stat3 (Tyr705) (D3A7)#9145, Stat3(124H6)#9139 were from Cell Signaling technology (Beverly, Mass., USA). Antibody against Fbxo40 #ab190688 was from Abcam (Cambridge, Mass., USA). Antibody against IRS1 #611395 was obtained from BD Biosciences (San Jose, Calif.). Anti-GAPDH#PA1-987 was from Thermo Fisher Scientific. The antibody was verified by the molecular size of recognized proteins.

Statistical Analysis: Student's t test was used when 2 experimental groups were compared and ANOVA when data from 3 or 4 groups were studied. After ANOVA analyses, pair wise comparisons were made by the Student-Newman-Keuls test. The data are presented as means±SEM.

Results

CKD Induces Insulin Resistance (IR) in Mice Via Stat3 Activation

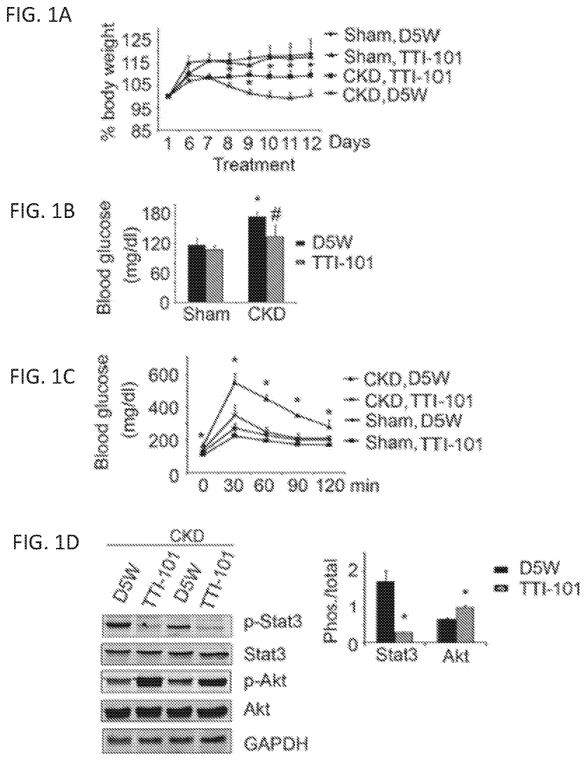

Earlier, it was determined that mice with CKD or cancer cachexia exhibit activation of Stat3 in muscle leading to muscle wasting.^{33,34} In those experiments, it was demonstrated that suppression of p-Stat3 following administration of a small molecule inhibitor, C188-9 (TTI-101), led to increases in body weights of mice despite the presence of CKD. In present studies, TTI-101 was administered every other day to mice with CKD. After 2 weeks of treatment, body weight increased and blood glucose levels decreased (FIGS. 1A and 1B). Treatment of mice with TTI-101 significantly improved the glucose tolerance in mice with CKD (FIG. 1C). Western blotting was performed of muscle lysates from the mice with CKD and found that TTI-101 treatment suppressed p-Stat3 while increasing p-Akt (FIG. 1D). The results indicate that CKD activates Stat3 in muscle causing insulin resistance in mice while inhibition of p-Stat3 using a small molecule inhibitor blocks these responses and could be tested for efficacy in reversing IR in patients.

Fbxo40 Expression is Induced by Stat3 Activation

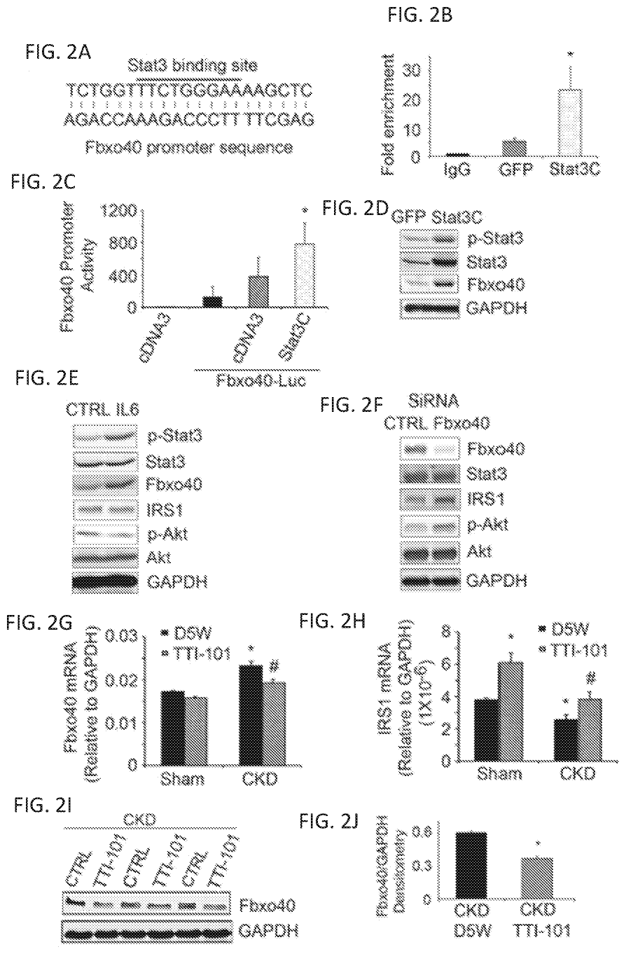

To determine downstream targets of Stat3, the expression was examined of the E3 ubiquitin ligase, Fbxo40, because a consensus Stat3 binding site sequence was identified within the Fbxo40 promoter (FIG. 2A). To test whether Stat3 binds to Fbxo40 promoter and stimulates its expression, CHIP and promoter activity assays were performed. First, C2C12 cells were transfected with plasmids that express constitutively active Stat3 (Stat3C); GFP-transfected cells were used as controls. Chromatins from these cells were immunoprecipitated with IgG or anti-Stat3 antibodies. DNAs isolated from the immunocomplexes were subjected to PCR analysis using primers from the Fbxo40 promoter that contain consensus sequences of the Stat3 binding site. The relative enrichment of Stat3 over IgG in cells expressing GFP or Stat3 indicates that Stat3 binds to Fbxo40 genes (FIG. 2B). Secondly, Fbxo40 promoter activity was measured. C2C12 myoblasts were transfected with plasmids that express Fbxo40-promoter-luciferase plus Stat3C or cDNA3 (as a control). After 24 hr, the cells were lysed by passive lysis buffer and Gaussia-luciferase activity was evaluated (see material Methods). Stat3C significantly increased Fbxo40 promoter activity (FIG. 2C). From Western blotting results, there were increases in Fbxo40 protein in cells transfected with Stat3C (FIG. 2D).

There are high circulating levels of IL-6 in mice or patients with CKD and IL-6 is known to stimulate Stat3 activation in muscle.^{33,35,36} To examine the physiological relevance of these IL-6 responses, C2C12 myotubes were treated with IL-6 and found it increased p-Stat3 and Fbxo⁴⁰ expression but decreased the IRS1 level and impaired Akt phosphorylation (FIG. 2E). Moreover, knockdown of Fbxo40 in C2C12 cells increased the protein levels of IRS1 and p-Akt even in myotubes treated with IL-6 (FIG. 2F). This is relevant because the SCF-Fbxo40 complex reportedly induces IRS1-ubiquitin conjugation in skeletal muscle leading to limitation of IGF1 signaling.²⁵ Stat3 activation stimulates Fbxo40 resulting in impaired insulin signaling.

Consistent with the increases in p-Stat3 in muscles of mice with CKD, Fbxo40 mRNAs were increased in muscles of mice with CKD (FIG. 2G). Notably, when p-Stat3 was inhibited in CKD mice using the small molecule inhibitor, TTI-101, there was suppression of the Fbxo40 mRNA (FIG. 2G). In contrast, in CKD mice, TTI-101 treatment increased IRS1 mRNAs compared to results from CKD mice that were treated with the diluent (D5W; FIG. 2H). Results of western blotting of tibialis anterior (TA) muscle lysates revealed that TTI-101 treatment of CKD mice significantly decreased Fbxo40 protein in the mouse muscles (FIGS. 2I and 2J). Stat3 activation induces IR by a pathway of Fbxo40-stimulated IRS1 degradation by the ubiquitin-proteasome system.

Stat3 Inhibition Improves HFD-Induced Insulin Resistance (IR) in Mice

To determine whether the IR that is induced by p-Stat3 activation represents a general mechanism, mice were studied with another type of IR, namely those fed a high-fat diet (HFD). After two weeks of the dietary regimen, there was increased muscle expression of p-Stat3 compared to results from mice eating standard chow (FIG. 3A). Mice fed the HFD exhibited increased muscle expression of the mRNA of Fbxo40 but not of Atrogin-1 or MuRF-1 (FIGS. 3B-3D). These results are relevant because activation of the E3 ubiquitin ligases, Atrogin-1 and MuRF-1, is highly associated with the development of muscle atrophy from degradation of muscle proteins. Thus, the activation of Fbxo40 does not represent a standardized response to dietary factors. These results were extended by feeding the HFD to another group of mice for 12 weeks (FIG. 3E); these mice developed glucose intolerance (FIG. 3F). Mice that had been fed the HFD for 12 weeks were divided into two subgroups and the HFD feeding was continued for another 4 weeks plus treatment with either TTI-101 or the diluent: one subgroup received injections of the diluent intraperitoneally while mice in the other subgroup was treated with intraperitoneal injections of TTI-101. Results from these studies included decreased fasting values of blood glucose in mice treated with TTI-101 vs. treatment with the diluent despite feeding the HFD (FIG. 3G). TTI-101 administration also improved glucose and insulin tolerances in HFD-mice (FIGS. 3H and 3I). Western blotting revealed that TTI-101 treatment of mice fed the HFD had higher levels of both IRS1 and p-Akt in muscles compared to results from mice fed the HFD and treated with the diluent (FIG. 3J). The results indicate that inhibition of p-Stat3 in HFD-mice led to an increase in insulin signaling pathway in muscles.

Stat3 KO in Muscles Suppresses HFD-Induced IR in Mice

In previous studies, it was determined that CKD impairs p-Akt in muscle and uncovered that inhibition of p-Stat3 improved the p-Akt³³. It is presently confirmed that Stat3 activation in muscles causes IR. To examine these relationships in another fashion, results were examined from mice with muscle-specific Stat3 KO that were created by crossing transgenic mice expressing floxed-Stat3 with MCK-Cre mice.^{33,34} The Stat3 KO and floxed-Stat3 mice were examined during 16 weeks of feeding the HFD. Comparing mice feeding with regular diet, HFD caused decrease in masses of muscle, but increase in adipose tissue. After 16 weeks HFD, in the two groups Stat3 KO and floxed-Stat3 mice, there were no significant differences in body (FIG. 4A) or muscle weights (FIG. 4B) or masses of adipose tissues (FIG. 4C). However, HFD feeding of muscle-specific Stat3 KO mice led to a substantial decrease in fasting blood glucose (FIG. 4D) plus improved glucose tolerances (FIGS. 4E and 4F). p-Stat3 in muscles plays an important role in the development of HFD-induced IR in mice.

Significance of Certain Embodiments

Previously, it was found that complications of CKD include increased protein degradation and impaired protein synthesis resulting in loss of muscle mass (FIG. 5).^{31,35} It was determined that the increase in protein degradation was mediated by stimulation of the ubiquitin-proteasome signaling pathway in muscles.³⁷ Specifically, two muscle-specific E3 ubiquitin ligases (Atrogin-1 and MuRF-1) were increased in muscles of rodents with CKD.²² In those experiments, p-Akt was impaired and IR developed³⁸. Currently, it is determined that activated Stat3 in muscle is associated with increased expression of the ubiquitin E3 ligase, Fbxo40. This was interesting because Fbxo40 induces both ubiquitin conjugation and degradation of the critical insulin-signaling molecule, IRS1.25 This response impairs the p-Akt level in muscles leading to the development of IR (FIG. 5). Additional support for a pathway from p-Stat3 to Fbxo40 to IR is that the inhibition of p-Stat3 by a small molecule inhibitor, TTI-101, improves insulin sensitivity both in mice with CKD as well as those with HFD-induced diabetes.

In studies of rodents with CKD, streptozotocin-induced acute diabetes or cancer cachexia, increased levels of p-Stat3 in muscle are associated with a Stat3/CEBPδ/myostatin pathway that is responsible for increasing muscle protein degradation. Notably, when p-Stat3 is inhibited with a small molecule inhibitor or when Stat3 KO is examined specifically in muscle, weights in body and muscle in mice with cancer or CKD were increased.^{33,34} Since IR occurs commonly in patients with CKD, diabetes or cancer cachexia, these disorders are explored to determine if Stat3 activation in muscles of mice with catabolic conditions is a key mediator that induces IR. In fact there is evidence that Stat3 activation develops into IR. The report by Mashili et al., indicated that Stat3 in skeletal muscles of patients with type 2 diabetes is constitutively phosphorylated ²¹. They also determined that silencing the Stat3 gene in myotubes prevents lipid-induced IR. The results in mice are consistent with these investigations. For example, it was found that glucose and insulin tolerances in mice with either CKD or type 2 diabetes were improved when p-Stat3 was inhibited with the small molecule inhibitor, TTI-101. When Stat3 is knocked out specifically in muscles of mice fed the HFD, muscle-specific KO of Stat3 exhibited improvement in glucose tolerances. The results differ from those of White et al. who studied mice with muscle-specific Stat3 KO by feeding them the HFD for 20 days. The investigators concluded that Stat3 KO and control mice exhibited similar phenotypes with no significant differences in measurements of fat mass, energy expenditure or whole-body fat oxidation. They concluded that Stat3 KO in skeletal muscles does not prevent a HFD-induced IR.³⁹ There are differences among the results of the investigation and those of White et al: the present inventors fed the HFD to mice with muscle-specific Stat3 KO or control mice for 16 weeks and documented an improvement in IR in Stat3 KO mice vs. results obtained from control mice. White et al. fed muscle-specific Stat3 KO mice for only 20 days and did not provide measurements of insulin or glucose levels.

Several cell-based mechanisms have been proposed to explain why IRS1 levels in muscles are low in diabetes; these include phosphotyrosine-dephosphorylation, serine-threonine phosphorylation and IRS1 degradation.⁴⁰⁻⁴² There also are reports suggesting that certain E3 ubiquitin ligases interact with IRS1 resulting in its proteasome-mediated degradation.^{43-45,37,44} For example, inflammation was found to stimulate activity of distinct E3 ubiquitin ligases, SOCS1 and SOCS3, which can interact with IRS1 or IRS2, leading to their degradation.44,45 Alternatively, the E3 ubiquitin ligase, Cbl-b, was found to be associated with degradation of IRS1 causing muscle atrophy.⁴⁶ Interestingly, Cbl-b activation was also found to induce the IR that results from feeding a HFD¹⁰. Finally, the cullin 7 complex, containing the E3 ubiquitin ligase, Fbxw8, can be activated by a mTOR-dependent, negative feedback mechanism that leads to the degradation of IRS land hence, causes IR.⁴⁷ Shi et. al reported that the E3 ubiquitin ligase, Fbxo⁴⁰, induces ubiquitin conjugation and degradation of IRS1 specifically in skeletal muscle cells and only in response to IGF1 stimulation.²⁵ There is demonstrated a sequence of changes from Stat3 to stimulation of Fbxo40 expression to IRS1 degradation.

In addition to increases in p-Stat3 in muscles of CKD or HFD-fed mice, reduced Akt phosphorylation was consistently observed in the same muscles. However, when p-Stat3 was inhibited with TTI-101, there was improved insulin sensitivity in both CKD and HFD mice as well as in mice with muscle-specific Stat3 KO. While exploring the molecular mechanism by which p-Stat3 stimulates IR, p-Stat3 potently induces Fbxo40 expression. The Stat3 to Fbxo40 to IRS1 pathway was confirmed when Fbxo40 was knocked down or when Stat3 was inhibited using TTI-101. These results strongly suggest that Fbxo40 is a mediator of p-Stat3 expression that leads to IR. Consistent with this conclusion, there was a greater increase in p-Stat3 and Fbxo40 protein levels in skeletal muscles of mice with CKD and that treatment of CKD mice with TTI-101 inhibited the expression of both p-Stat3 and Fbxo40 while increasing p-Akt.

For the first time, it is uncovered how CKD or HFD induces IR: Stat3 activation causes IRS1 degradation and hence, IR. The mediator of these changes is up-regulation of the ubiquitin E3 ligase, Fbxo40. Because others report that inflammation induces IR, the results have uncovered a general mechanism by which Stat3 influences responses to other disorders such as type two diabetes, obesity and cardiovascular diseases. The results provide a foundation for designing a clinical strategy directed at targeting Stat3 to treat IR arising from complex disorders such as inflammation.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

1 DeFronzo R A, Beckles A D: Glucose intolerance following chronic metabolic acidosis in man. Am J Physiol 1979; 236:E328-E334.
2 DeFronzo R A, Tobin J D, Rowe J W, Andres R: Glucose intolerance in uremia: Quantification of pancreatic beta cell sensitivity to glucose and tissue sensitivity to insulin. J Clin Invest 1978; 62:425-435.
3 DeFronzo R A, Alvestrand A, Smith D, Hendler R: Insulin resistance in uremia. J Clin Invest 1981; 67:563-568.
4 Fliser D, Pacini G, Engelleiter R, Kautzky-Willer A, Prager R, Franek E, Ritz E: Insulin resistance and hyperinsulinemia are already present in patients with incipient renal disease. Kidney Int 1998; 53:1343-1347.
5 Spoto B, Pisano A, Zoccali C: Insulin resistance in chronic kidney disease: a systematic review. Am J Physiol Renal Physiol 2016; 311:F1087-F1108.
6 Liao M T, Sung C C, Hung K C, Wu C C, Lo L, Lu K C: Insulin resistance in patients with chronic kidney disease. J Biomed Biotechnol 2012; 2012:691369.
7 de Boer I H, Mehrotra R: Insulin resistance in chronic kidney disease: a step closer to effective evaluation and treatment. Kidney Int 2014; 86:243-245.
8 Shoelson S E, Lee J, Goldfine A B: Inflammation and insulin resistance. J Clin Invest 2006; 116:1793-1801.
9 Ferris H A, Kahn C R: New mechanisms of glucocorticoid-induced insulin resistance: make no bones about it. J Clin Invest 2012; 122:3854-3857.
10 Bonala S, Lokireddy S, McFarlane C, Patnam S, Sharma M, Kambadur R: Myostatin induces insulin resistance via Casitas B-lineage lymphoma b (Cblb)-mediated degradation of insulin receptor substrate 1 (IRS1) protein in response to high calorie diet intake. J Biol Chem 2014; 289:7654-7670.
11 Kim J H, Bachmann R A, Chen J: Interleukin-6 and insulin resistance. Vitam Horm 2009; 80:613-633.
12 Arkan M C, Hevener A L, Greten F R, Maeda S, Li Z W, Long J M, Wynshaw-Boris A, Poli G, Olefsky J, Karin M: IKK-beta links inflammation to obesity-induced insulin resistance. Nat Med 2005; 11:191-198.
13 Kim J H, Yoon M S, Chen J: Signal transducer and activator of transcription 3 (STAT3) mediates amino acid inhibition of insulin signaling through serine 727 phosphorylation. J Biol Chem 2009; 284:35425-35432.
14 de Castro B T, de Carvalho J E, Poyares L L, Bordin S, Machado U F, Nunes M T: Potential role of growth hormone in impairment of insulin signaling in skeletal muscle, adipose tissue, and liver of rats chronically treated with arginine. Endocrin 2009; 150:2080-2086.
15 Schindler C, Levy D E, Decker T: JAK-STAT signaling: from interferons to cytokines. J Biol Chem 2007; 282:20059-20063.
16 Krebs D L, Hilton D J: SOCS: physiological suppressors of cytokine signaling. J Cell Sci 2000; 113 (Pt 16):2813-2819.
17 Krebs D L, Hilton D J: A new role for SOCS in insulin action. Suppressor of cytokine signaling. Sci STKE 2003; 2003:E6.
18 Ueki K, Kondo T, Kahn C R: Suppressor of cytokine signaling 1 (SOCS-1) and SOCS-3 cause insulin resistance through inhibition of tyrosine phosphorylation of insulin receptor substrate proteins by discrete mechanisms. Mol Cell Biol 2004; 24:5434-5446.
19 Torisu T, Sato N, Yoshiga D, Kobayashi T, Yoshioka T, Mori H, lida M, Yoshimura A: The dual function of hepatic SOCS3 in insulin resistance in vivo. Genes Cells 2007; 12:143-154.
20 Howard J K, Flier J S: Attenuation of leptin and insulin signaling by SOCS proteins. Trends Endocrinol Metab 2006; 17:365-371.
21 Mashili F, Chibalin A V, Krook A, Zierath J R: Constitutive STAT3 phosphorylation contributes to skeletal muscle insulin resistance in type 2 diabetes. Diabetes 2013; 62:457-465.
22 Lee S W, Dai G, Hu Z, Wang X, Du J, Mitch W E: Regulation of muscle protein degradation: coordinated control of apoptotic and ubiquitin-proteasome systems by phosphatidylinositol 3 kinase. J Am Soc Nephrol 2004; 15:1537-1545.
23 Sandri M, Sandri C, Gilbert A, Skuck C, Calabria E, Picard A, Walsh K, Schiaffino S, Lecker S H, Goldberg A L: Foxo transcription factors induce the atrophy-related ubiquitin ligase atrogin-1 and cause skeletal muscle atrophy. Cell 2004; 117:399-412.
24 Ho M S, Tsai P I, Chien C T: F-box proteins: the key to protein degradation. J Biomed Sci 2006; 13:181-191.
25 Shi J, Luo L, Eash J, Ibebunjo C, Glass D J: The SCF-Fbxo40 complex induces IRS1 ubiquitination in skeletal muscle, limiting IGF1 signaling. Dev Cell 2011; 21:835-847.
26 Ye J, Zhang Y, Xu J, Zhang Q, Zhu D: FBXO40, a gene encoding a novel muscle-specific F-box protein, is upregulated in denervation-related muscle atrophy. Gene 2007; 404:53-60.
27 Carvalho E, Jansson P A, Axelsen M, Eriksson J W, Huang X, Groop L, Rondinone C, Sjostrom L, Smith U: Low cellular IRS 1 gene and protein expression predict insulin resistance and NIDDM. FASEB J 1999; 13:2173-2178.
28 Araki E, Lipes M A, Patti M-E, Bruning J C, Haag B, Johnson R S, Kahn C R: Alternative pathway of insulin signalling in mice with targeted disruption of the IRS-1 gene. Nature 1994; 372:186-190.
29 Tamemoto H, Kadowaki T, Tobe K, Yagi T, Sakura H, Hayakawa T, Terauchi Y, Ueki K, Kaburagi Y, Satoh S,: Insulin resistance and growth retardation in mice lacking insulin receptor substrate-1. Nature 1994; 372:182-186.
30 Bruning J C, Winnay J, Bonner-Weir S, Taylor S I, Accili D, Kahn C R: Development of a novel polygenic model of NIDDM in mice heterozygous for IR and IRS-1 null alleles. Cell 1997; 88:561-572.
31 Zhang L, Rajan V, Lin E, Hu Z, Han H Q, Zhou X, Song Y, Min H, Wang X, Du J, Mitch WE: Pharmacological inhibition of myostatin suppresses systemic inflammation and muscle atrophy in mice with chronic kidney disease. FASEB J 2011; 25:1653-1663.
32 Bharadwaj U, Eckols T K, Xu X, Kasembeli M M, Chen Y, Adachi M, Song Y, Mo Q, Lai S Y, Tweardy D J: Small-molecule inhibition of STAT3 in radioresistant head and neck squamous cell carcinoma. Oncotarget 2016; 7:26307-26330.
33 Zhang L, Pan J, Dong Y, Tweardy D J, Dong Y, Garibotto G, Mitch W E: Stat3 Activation Links a C/EBPdelta to Myostatin Pathway to Stimulate Loss of Muscle Mass. Cell Metab 2013; 18:368-379.
34 Silva K A, Dong J, Dong Y, Dong Y, Schor N, Tweardy D J, Zhang L, Mitch W E: Inhibition of Stat3 activation suppresses caspase-3 and the ubiquitin-proteasome system, leading to preservation of muscle mass in cancer cachexia. J Biol Chem 2015; 290:11177-11187.
35 Zhang L, Du J, Hu Z, Han G, Delafontaine P, Garcia G, Mitch W E: IL-6 and serum amyloid A synergy mediates angiotensin II-induced muscle wasting. J Am Soc Nephrol 2009; 20:604-612.
36 Zhang L, Wang X H, Wang H, Du J, Mitch W E: Satellite cell dysfunction and impaired IGF-1 signaling cause CKD-induced muscle atrophy. J Am Soc Nephrol 2010; 21:419-427.
37 Mitch W E, Goldberg A L: Mechanisms of muscle wasting: The role of the ubiquitin-proteasome system. N Engl J Med 1996; 335:1897-1905.
38 Bailey J L, Price S R, Zheng B, Hu Z, Mitch W E: Chronic kidney disease causes defects in signaling through the insulin receptor substrate/phosphatidylinositol 3-kinase/Akt pathway: implications for muscle atroply. J Am Soc Nephrol 2006; 17:1388-1394.
39 White A T, LaBarge S A, McCurdy C E, Schenk S: Knockout of STAT3 in skeletal muscle does not prevent high-fat diet-induced insulin resistance. Mol Metab 2015; 4:569-575.
40 Aguirre V, Werner E D, Giraud J, Lee Y H, Shoelson S E, White M F: Phosphorylation of Ser 307 in insulin receptor substrate-1 blocks interactions with the insulin receptor and inhibits insulin action. J Biol Chem 2002; 277:1531-1537.
41 Rui L, Aguirre V, Kim J K, Shulman G I, Lee A, Corbould A, Dunaif A, White M F: Insulin/IGF-1 and TNF-a stimulate phosphorylation of IRS-1 at inhibitory Ser307 via distinct pathways. J Clin Invest 2001; 107:181-189.
42 Pederson T M, Kramer D L, Rondinone C M: Serine/threonine phosphorylation of IRS-1 triggers its degradation: possible regulation by tyrosine phosphorylation. Diabetes 2001; 50:24-31.
43 Myers M G, White M F: Insulin signal transduction and the IRS proteins. Ann Rev Pharmacol Toxicol 1996; 36:615-658.
44 Rui L, Fisher T L, Thomas J, White M F: Regulation of insulin/insulin-like growth factor-1 signaling by proteasome-mediated degradation of insulin receptor substrate-1. J Biol Chem 2004; 276:40362-40367.
45 Rui L, Yuan M, Frantz D, Shoelson S, White MF: SOCS-1 and SOCS-3 block insulin signaling by ubiquitin-mediated degradation of IRS1 and IRS2. J Biol Chem 2002; 277:42394-42398.
46 Nakao R, Hirasaka K, Goto J, Ishidoh K, Yamada C, Ohno A, Okumura Y, Nonaka I, Yasutomo K, Baldwin K M, Kominami E, Higashibata A, Nagano K, Tanaka K, Yasui N, Mills E M, Takeda S, Nikawa T: Ubiquitin ligase Cbl-b is a negative regulator for insulin-like growth factor 1 signaling during muscle atrophy caused by unloading. Mol Cell Biol 2009; 29:4798-4811.
47 Xu X, Sarikas A, Dias-Santagata D C, Dolios G, Lafontant P J, Tsai S C, Zhu W, Nakajima H, Nakajima H O, Field L J, Wang R, Pan Z Q: The CULT E3 ubiquitin ligase targets insulin receptor substrate 1 for ubiquitin-dependent degradation. Mol Cell 2008; 30:403-414.

What is claimed is:

1. A method of treating, preventing, or reducing the risk or severity of insulin resistance or a condition associated with insulin resistance in an individual in need thereof, comprising administering to the individual a therapeutically effective amount of one or more inhibitors of signal transducer and activator of transcription 3 (STAT3).

2. The method of claim 2, wherein the insulin resistance in the individual is associated with inflammation.

3. The method of claim 1 or 2, wherein the individual has chronic kidney disease (CKD).

4. The method of any one of claims 1-3, wherein the individual has diabetes.

5. The method of any one of claims 1-4, wherein the individual has obesity.

6. The method of any one of claims 1-5, wherein the individual has a cardiovascular disease or disorder.

7. The method of any one of claims 1-6, wherein the individual does not have cachexia or muscle wasting.

8. The method of any one of claims 1-7, wherein the condition associated with insulin resistance is severe high blood sugar, severe low blood sugar, heart attack; stroke, kidney disease, eye problems, cancer, non-alcoholic fatty liver disease (NAFLD), polycystic ovarian syndrome (PCOS), metabolic syndrome, diabetes, or Alzheimer's disease.

9. A method of treating, preventing, or reducing the risk or severity of diabetes in an individual in need thereof, comprising administering to the individual a therapeutically effective amount of one or more inhibitors of signal transducer and activator of transcription 3 (STAT3).

10. The method of claim 9, wherein the diabetes is Type II diabetes.

11. A method of treating, preventing, or reducing the risk or severity of metabolic syndrome in an individual in need thereof, comprising administering to the individual a therapeutically effective amount of one or more inhibitors of signal transducer and activator of transcription 3 (STAT3).

12. The method of claim 11, wherein the metabolic syndrome comprises risk factors associated with diabetes and cardiovascular disease or disorder.

13. The method of claim 12, wherein the risk factors comprise high blood triglycerides, high blood pressure, belly fat, and high blood sugar, and low high-density lipoprotein (HDL) cholesterol level.

14. The method of any one of claims 1-13, wherein the individual is a mammal.

15. The method of claim 14, wherein the mammal is a human.

16. The method of any one of claims 1-15, wherein the inhibitor of STAT3 is a small molecule.

17. The method of any one of claims 1-16, wherein the inhibitor of STAT3 is one or more inhibitors from any one of Tables 1-7, or a pharmaceutically acceptable salt thereof.

18. The method of any one of claims 1-16, wherein the inhibitor of STAT3 is selected from the group consisting of N-(1′,2-dihydroxy-1,2′-binaphthalen-4′-yl)-4-methoxybenzenesulfonamide, N-(3,1′-Dihydroxy-[1,2]binaphthalenyl-4′-yl)-4-methoxy-benzenesulfonamide, N-(4,1′-Dihydroxy-[1,2]binaphthalenyl-4′-yl)-4-methoxy-benzenesulfonamide, N-(5,1′-Dihydroxy-[1,2]binaphthalenyl-4′-yl)-4-methoxy-benzenesulfonamide, N-(6,1′-Dihydroxy-[1,2]binaphthalenyl-4′-yl)-4-methoxy-benzenesulfonamide, N-(7,1′-Dihydroxy-[1,2]binaphthalenyl-4′-yl)-4-methoxy-benzenesulfonamide, N-(8,1′-Dihydroxy-[1,2]binaphthalenyl-4′-yl)-4-methoxy-benzenesulfonamide, 4-Bromo-N-(1,6′-dihydroxy-[2,2]binaphthalenyl-4-yl)-benzenesulfonamide, and 4-Bromo-N-[4-hydroxy-3-(1H-[1,2,4]triazol-3-ylsulfanyl)-naphthalen-1-yl]-benzenesulfonamide, or a pharmaceutically acceptable salt thereof. cm 19. The method of any one of claims 1-16, wherein the inhibitor of STAT3 is N-(1′,2-dihydroxy-1,2′-binaphthalen-4′-yl)-4-methoxybenzenesulfonamide, or a pharmaceutically acceptable salt thereof.

20. The method of any one of claims 1-16, wherein the inhibitor of STAT3 is a compound of Formula IV: 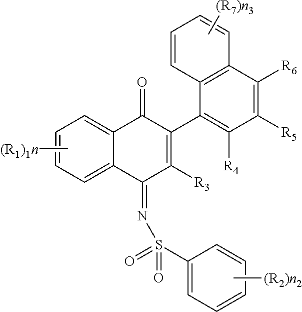
or a pharmaceutically acceptable salt thereof, wherein
each occurrence of R₁ is independently hydrogen, halogen, cyano, nitro, CF₃, OCF₃, ORₐ, SRₐ, C(═O)Rₐ, OC(═O)Rₐ, C(═O)ORₐ, NR_{b}R_{c}, NR_{b}C(═O)R_{c}, C(═O)NR_{b}R_{c}, NR_{b}C(═O)OR_{c}, OC(═O)NR_{b}R_{c}, NRₐC(═O)NR_{b}R_{c}, alkyl, alkenyl, cycloalkyl, optionally substituted aryl, or optionally substituted heterocycle;
m is 0, 1, 2, 3, or 4;
each occurrence of R2 is independently hydrogen, halogen, cyano, nitro, CF₃, OCF₃, ORₐ, SRₐ, C(═O)Rₐ, OC(═O)Rₐ, C(═O)ORₐ, NR_{b}R_{c}, NR_{b}C(═O)R_{c}, C(═O)NR_{b}R_{c}, NR_{b}C(═O)OR_{c}, OC(═O)NR_{b}R_{c}, NRₐC(═O)NR_{b}R_{c}, alkyl, alkenyl, cycloalkyl, cycloalkenyl, optionally substituted aryl, optionally substituted aryloxyl, or optionally substituted heterocycle;
n₂ is 0, 1, 2, 3, 4, or 5;
R₃ is hydrogen, halogen, cyano, nitro, CF₃, OCF₃, ORₐ, SRₐ, OC(═O)Rₐ, alkyl, alkenyl, cycloalkyl, or optionally substituted aryl or heteroaryl;
R₄ is hydrogen, halogen, cyano, nitro, CF₃, OCF₃, ORₐ, SRₐ, NR_{b}R_{c}, OC(═O)Rₐ, alkyl, alkenyl, or cycloalkyl;
each occurrence of R₅, R₆, and R₇ is independently hydrogen, halogen, cyano, nitro, CF₃, OCF₃, ORₐ, SRₐ, C(═O)Rₐ, OC(═O)Rₐ, C(═O)ORₐ, NR_{b}R_{c}, NR_{b}C(═O)R_{c}, C(═O)NR_{b}R_{c}, NR_{b}C(═O)OR_{c}, OC(═O)NR_{b}R_{c}, NRₐC(═O)NR_{b}R_{c}, alkyl, alkenyl, cycloalkyl, optionally substituted aryl, or optionally substituted heterocycle;
n3 is 0, 1, 2, 3, or 4; and
each occurrence of Rₐ, R_{b}, and R_{c} is independently hydrogen, alkyl, cycloalkyl, alkenyl, cycloalkenyl, alkynyl, heterocycle, or aryl; or said R_{b} and R_{c} together with the nitrogen atom to which they are bonded optionally form a heterocycle comprising 1-4 heteroatoms.

21. The method of claim 20, wherein the compound is a compound of Formula V: 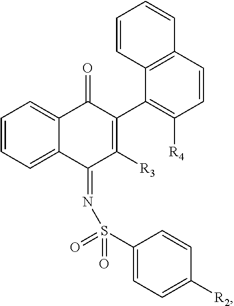
or a pharmaceutically acceptable salt thereof.

22. The method of claim 21, wherein the compound is a compound of Formula VI: 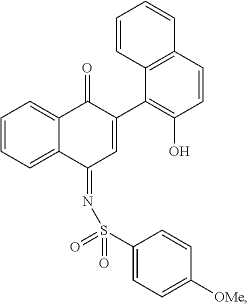 [SMILES: COc1ccc(S(=O)(=O)/N=C2\C=C(c3c(O)ccc4ccccc34)C(=O)c3ccccc32)cc1]
or a pharmaceutically acceptable salt thereof.

23. The method of any one of claims 1-22, further comprising administering to the individual an additional therapy.
